(19) United States
(12) Patent Application Publication
(10) Pub. No.: US 2018/0280528 A1
(43) Pub. Date: Oct. 4, 2018

(54) ANTIFOLATE CONJUGATES FOR TREATING INFLAMMATION

(71) Applicant: ENDOCYTE, INC., West Lafayette, IN (US)

(72) Inventors: Iontcho Radoslavov VLAHOV, West Lafayette, IN (US); Christopher Paul LEAMON, West Lafayette, IN (US); Fei YOU, West Lafayette, IN (US); Yingjuan J. LU, West Lafayette, IN (US); Paul Joseph KLEINDL, Lebanon, IN (US)

(21) Appl. No.: 15/570,605

(22) PCT Filed: Apr. 29, 2016
(86) PCT No.: PCT/US16/30150

§ 371 Date: Oct. 30, 2017

Related U.S. Application Data
(60) Provisional application No. 62/155,805, filed on May 1, 2015.

(51) Int. Cl. A61K 47/65; A61K 47/55; A61K 31/519; A61P 29/00; A61P 19/02

(52) U.S. Cl. CPC A61K 47/65; A61K 47/551; A61P 19/02; A61P 29/00; A61K 31/519

(57) ABSTRACT

The present invention relates to compositions and methods for use in targeted drug delivery. More particularly, the invention is directed to cell-surface receptor binding conjugates containing hydrophilic spacer linkers for use in treating disease states caused by pathogenic cell populations and to methods and pharmaceutical compositions that use and include such conjugates.

### CROSS REFERENCE TO RELATED APPLICATIONS

This application claims priority under 35 U.S.C. § 119(e) to U.S. Provisional Application Ser. No. 62/155,805, filed May 1, 2015, which is incorporated herein by reference in its entirety.

### TECHNICAL FIELD

The present invention relates to compositions and methods for use in targeted drug delivery. More particularly, the invention is directed to cell-surface receptor binding conjugates containing hydrophilic spacer linkers for use in treating disease states caused by pathogenic cell populations and to methods and pharmaceutical compositions that use and include such conjugates.

### BACKGROUND

The mammalian immune system provides a means for the recognition and elimination of foreign pathogens. While the immune system normally provides a line of defense against foreign pathogens, there are many instances where the immune response itself is involved in the progression of disease. Exemplary of diseases caused or worsened by an immune response are autoimmune diseases and other diseases in which the immune response contributes to pathogenesis. For example, macrophages are generally the first cells to encounter foreign pathogens, and accordingly, they play an important role in the immune response, but activated macrophages can also contribute to the pathophysiology of disease in some instances.

The folate receptor is a 38 KD GPI-anchored protein that binds the vitamin folic acid with high affinity (<1 nM). Following receptor binding, rapid endocytosis delivers the vitamin into the cell, where it is unloaded in an endosomal compartment at low pH. Importantly, covalent conjugation of small molecules, proteins, and even liposomes to folic acid does not block the vitamin's ability to bind the folate receptor, and therefore, folate-drug conjugates can readily be delivered to and can enter cells by receptor-mediated endocytosis.

Because most cells use an unrelated reduced folate carrier to acquire the necessary folic acid, expression of the folate receptor is restricted to a few cell types. With the exception of kidney, choroid plexus, and placenta, normal tissues express low or nondetectable levels of the folate receptor. It has been reported that the folate receptor β, the nonepithelial isoform of the folate receptor, is expressed on activated (but not resting) synovial macrophages. Thus, folate receptors are expressed on a subset of macrophages (i.e., activated macrophages). Folate receptors of the β isoform are also found on activated monocytes.

Accordingly, the present invention relates to the development of vitamin-targeted therapeutics, such as folate-targeted therapeutics, to treat inflammation. The folate conjugates described herein can be used to treat inflammatory diseases by targeting inflammatory cells that overexpress the folate receptor.

### SUMMARY

In one aspect, the disclosure provides conjugates of the formula B-L-D¹, wherein B is a binding ligand, L is a linker comprising a releaseable linker (L¹), at least one AA, and at least one L¹, and D¹ is a drug; wherein B, D¹, L¹, L² and AA are defined as described herein in various embodiments and examples; or a pharmaceutically acceptable salt thereof.

In another aspect, the disclosure provides conjugates of the formula B-L-D¹, wherein B is a binding ligand as described herein, L is a linker comprising at least one AA as described herein, at least one L¹ as described herein and an L² as described herein, or a pharmaceutically acceptable salt thereof.

In some embodiments, the disclosure provides a conjugate of the formula B-L¹-AA-L¹-AA-L¹-L²-D¹, B-AA-L¹-AA-AA-L²-D¹, or B-AA-AA-AA-AA-L²-D¹, wherein B, AA, L¹, L² and D¹ are as described herein; or a pharmaceutically acceptable salt thereof.

In some embodiments, the disclosure provides a conjugate of the formula B-L-D¹, wherein B is a binding ligand of the formula

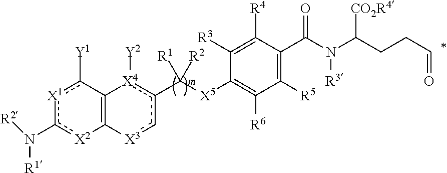

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR⁰ or —C(O)NR¹⁰R^{10′};

each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;

X¹ is —NR¹¹—, ═N—, —N═, —C(R¹¹)═ or ═C(R¹¹)—;

X² is —NR^{11′}— or ═N—;

X³ is —NR^{11″}—, —N═ or —C(R^{11′})═;

X⁴ is —N═ or —C═;

X⁵ is NR¹² or CR¹²R^{12′};

Y¹ is H, D, —OR¹³ or —SR¹³ when X¹ is —N═ or —C(R¹¹)═, or Y¹ is ═O when X¹ is —NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;

R^{1′}, R^{2′}, R^{3′}, R^{4′}, R¹¹, R^{11′}, R^{11″}, R¹², R^{12′}, R¹³, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

L is a linker comprising at least one AA, at least one L¹ and an L², wherein each AA is an amino acid, each L¹ is of the formula

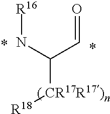

wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

and L² is of the formula

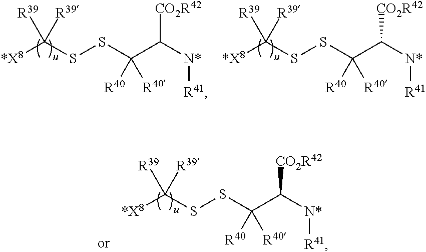

wherein

X⁸ is —NR⁵⁰— or —O—;

each R³⁹, R^{39′}, R⁴⁰ and R^{40′} is independently selected from the group consisting of H, D, C₁-C₆ alkyl, C₂-C₆ alkenyl, C₂-C₆ alkynyl, C₃-C₆ cycloalkyl, —OR⁴⁸, —OC(O)R⁴⁸, —OC(O)NR⁴⁸R^{48′}, —OS(O)R⁴⁸, —OS(O)₂R⁴⁸, —SR⁴⁸, —S(O)R⁴⁸, —S(O)₂R⁴⁸, —S(O)NR⁴⁸R^{48′}, —S(O)₂NR⁴⁸R^{48′}, —OS(O)NR⁴⁸R^{48′}, —OS(O)₂NR⁴⁸R^{48′}, —NR⁴⁸R^{48′}, —NR⁴⁸C(O)R⁴⁹, —NR⁴⁸C(O)OR⁴⁹, —NR⁴⁸C(O)NR⁴⁹R^{49′}, —NR⁴⁸S(O)R⁴⁹, —NR⁴⁸S(O)₂R⁴⁹, —NR⁴⁸S(O)NR⁴⁹R^{49′}, —NR⁴⁸S(O)₂NR⁴⁹R^{49′}, —C(O)R⁴⁸, —C(O)OR⁴⁸ or —C(O)NR⁴⁸R^{48′}, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR⁴⁴, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};

each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};

each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′}; and

each R⁴³, R^{43′}, R⁴⁴, R^{44′}, R⁴⁵, R^{45′}, R⁴⁶, R^{46′}, R⁴⁷, R^{47′}, R⁴⁸, R^{48′}, R⁴⁹, R^{49′} and R⁵⁰ is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl; and

u is 1, 2, 3 or 4; and

D¹ is a drug of the formula

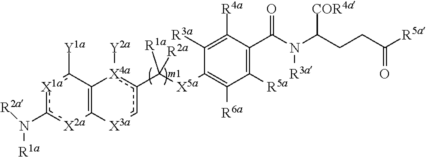

wherein

R^{1a} and R^{2a} in each instance are independently selected from the group consisting of H, D, halogen, C₁-C₆ alkyl, C₂-C₆ alkenyl, C₂-C₆ alkynyl, —OR^{7a}, —SR^{7a} and —NR^{7a}R^{7a′}, wherein each hydrogen atom in C₁-C₆ alkyl, C₂-C₆ alkenyl and C₂-C₆ alkynyl is independently optionally substituted by halogen, —OR^{8a}, —SR^{8a}, —NR^{8a}R^{8a′}, —C(O)R^{8a}, —C(O)OR^{8a} or —C(O)NR^{8a}R^{8a′};

R^{3a}, R^{4a}, R^{5a} and R^{6a} are each independently selected from the group consisting of H, D, halogen, C₁-C₆ alkyl, C₂-C₆ alkenyl, C₂-C₆ alkynyl, —CN, —NO₂, —NCO, —OR^{9a}, —SR^{9a}, —NR^{9a}R^{9a′}, —C(O)R^{9a}, —C(O)OR^{9a} and —C(O)NR^{9a}R^{9a′}, wherein each hydrogen atom in C₁-C₆ alkyl, C₂-C₆ alkenyl and C₂-C₆ alkynyl is independently optionally substituted by halogen, —OR^{10a}, —SR^{10a}, —NR^{10a}R^{10a′}, —C(O)R^{10a}, —C(O)OR^{10a} or —C(O)NR^{10a}R^{10a′};

each R^{7a}, R^{7a′}, R^{8a}, R^{8a′}, R^{9a}, R^{9a′}, R^{10a} and R^{10a′} is independently H, D, C₁-C₆ alkyl, C₂-C₆ alkenyl or C₂-C₆ alkynyl;

X^{1a} is —NR^{11a}—, ═N—, —N═, —C(R^{11a})═ or ═C(R^{11a})—;

X^{2a} is —NR^{11a′}— or ═N—;

X^{3a} is —NR^{11a″}—, —N═ or —C(R^{11a′})═;

X^{4a} is —N═ or —C═;

X^{5a} is —NR^{12a}— or —CR^{12a}R^{12a′}—;

Y^{1a} is —NR^{13a}R^{13a′} when X^{1a} is —N═ or —C(R^{11a})═, or Y^{1a} is ═NR^{13a} when X^{1a} is —NR^{11a}—, ═N— or ═C(R^{11a})—;

Y^{2a} is H, D, C₁-C₆ alkyl, C₂-C₆ alkenyl, —C(O)R^{14a}, —C(O)OR^{14a} or —C(O)NR^{14a}R^{14a′} when X^{4a} is —C═, or Y^{2a} is absent when X^{4a} is —N═;

R^{1a′}, R^{2a′}, R^{3a′}, R^{11a}, R^{11a′}, R^{11a″}, R^{12a}, R^{12a′}, R^{13a}, R^{13a′}, R^{14a} and R^{14a′} are each independently selected from the group consisting of H, D, C₁-C₆ alkyl, C₂-C₆ alkenyl, C₂-C₆ alkynyl, —C(O)R^{15a}, —C(O)OR^{15a} and —C(O)NR^{15a}R^{15a′};

R^{4a′} and R^{5a′} are each independently selected from the group consisting of C₁-C₆ alkyl, C₂-C₆ alkenyl, C₂-C₆ alkynyl, —OR^{16a}, —SR^{16a}, —NR^{16a}R^{16a′}, provided that one of R^{4a′} and R^{5a′} is a covalent bond to an AA, a L¹ or a L²;

R^{15a}, R^{15a′}, R^{16a} and R^{16a′} are each independently H or C₁-C₆ alkyl;

m¹ is 1, 2, 3 or 4; and

each * is a covalent bond;

or a pharmaceutically acceptable salt thereof.

In some embodiments, the disclosure provides a conjugate of the formula B-L-D¹, wherein B is a binding ligand of the formula

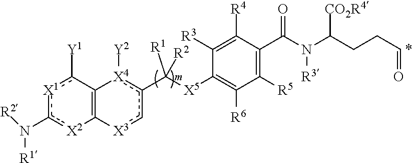

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;

X¹ is —NR¹¹—, ═N—, —N═, —C(R¹¹)═ or ═C(R¹¹)—;

X² is —NR^{11′}— or ═N—;

X³ is —NR^{11″}—, —N═ or —C(R^{11′})═;

X⁴ is —N═ or —C═;

X⁵ is NR¹² or CR¹²R^{12′};

Y¹ is H, D, —OR¹³ or —SR¹³ when X¹ is —N═ or —C(R¹¹)═, or Y¹ is ═O when X¹ is —NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;

R^{1′}, R^{2′}, R^{3′}, R^{4′}, R¹¹, R^{11′}, R^{11″}, R¹², R^{12′}, R¹³, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

L is a linker comprising at least one AA, at least one L¹ and an L², wherein each AA is an amino acid, each L¹ is of the formula

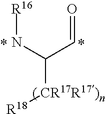

wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R²⁶, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5; and

L² is of the formula

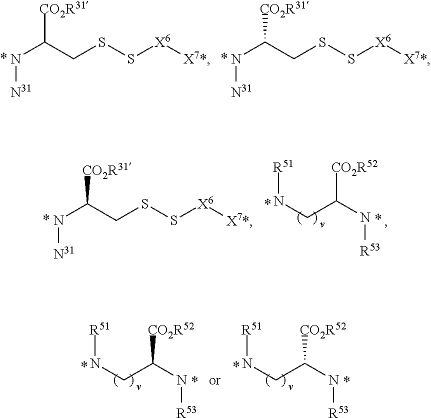

wherein

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};

each X⁷ is —NR^{31a}— or —O—, and when X⁶ is C₁-C₆ alkyl and X⁷ is —O—, then at least one hydrogen atom in C₁-C₆ alkyl is substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};

each R³¹ and R^{31a} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a}, —OS(O)₂NR^{32a}R^{32a′}, —NR^{32a}R^{32a′}, —C(O)R^{32a}, —C(O)OR^{32a} or —C(O)NR^{32a}R^{32a′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

each R⁵¹ and R⁵³ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁴, —OC(O)R⁵⁴, —OC(O)NR⁵⁴R^{54′}, —OS(O)R⁵⁴, —OS(O)₂R⁵⁴, —SR⁵⁴, —S(O)R⁵⁴, —S(O)₂R⁵⁴, —S(O)NR⁵⁴R^{54′}, —S(O)₂NR⁵⁴R^{54′}, —OS(O)NR⁵⁴R^{54′}, —OS(O)₂NR⁵⁴R^{54′}, —NR⁵⁴R^{54′}, —NR⁵⁴C(O)R⁵⁵, —NR⁵⁴C(O)OR⁵⁵, —NR⁵⁴C(O)NR⁵⁵R^{55′}, —NR⁵⁴S(O)R⁵⁵, —NR⁵⁴S(O)₂R⁵⁵, —NR⁵⁴S(O)NR⁵⁵R^{55′}, —NR⁵⁴S(O)₂NR⁵⁵R^{55′}, —C(O)R⁵⁴, —C(O)OR⁵⁴ or —C(O)NR⁵⁴R^{54′};

each R⁵² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁵⁶, —OC(O)R⁵⁶, —OC(O)NR⁵⁶R^{56′}, —OS(O)R⁵⁶, —OS(O)₂R⁵⁶, —SR⁵⁶, —S(O)R⁵⁶, —S(O)₂R⁵⁶, —S(O)NR⁵⁶R^{56′}, —S(O)₂NR⁵⁶R^{56′}, —OS(O)NR⁵⁶R^{56′}, —OS(O)₂NR⁵⁶R^{56′}, —NR⁵⁶R^{56′}, —C(O)R⁵⁶, —C(O)OR⁵⁶ or —C(O)NR⁵⁶R^{56′};

each R⁵⁴, R^{54′}, R⁵⁵, R^{55′}, R⁵⁶ and R^{56′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl; and

v is 1, 2, 3, 4, 5 or 6; and

D¹ is a drug of the formula I

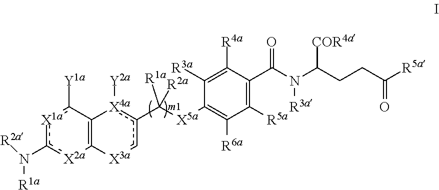

wherein

R^{1a} and R^{2a} in each instance are independently selected from the group consisting of H, D, halogen, C₁-C₆ alkyl, C₂-C₆ alkenyl, C₂-C₆ alkynyl, —OR^{7a}, —SR^{7a} and —NR^{7a}R^{7a′}, wherein each hydrogen atom in C₁-C₆ alkyl, C₂-C₆ alkenyl and C₂-C₆ alkynyl is independently optionally substituted by halogen, —OR^{8a}, —SR^{8a}, —NR^{8a}R^{8a′}, —C(O)R^{8a}, —C(O)OR^{8a} or —C(O)NR^{8a}R^{8a′};

R^{3a}, R^{4a}, R^{5a} and R^{6a} are each independently selected from the group consisting of H, D, halogen, C₁-C₆ alkyl, C₂-C₆ alkenyl, C₂-C₆ alkynyl, —CN, —NO₂, —NCO, —OR^{9a}, —SR^{9a}, —NR^{9a}R^{9a′}, —C(O)R^{9a}, —C(O)OR^{9a} and —C(O)NR^{9a}R^{9a′}, wherein each hydrogen atom in C₁-C₆ alkyl, C₂-C₆ alkenyl and C₂-C₆ alkynyl is independently optionally substituted by halogen, —OR^{10a}, —SR^{10a}, —NR^{10a}R^{10a′}, —C(O)R^{10a}, —C(O)OR^{10a} or —C(O)NR^{10a}R^{10a′};

each R^{7a}, R^{7a′}, R^{8a}, R^{8a′}, R^{9a}, R^{9a′}, R^{10a} and R^{10a′} is independently H, D, C₁-C₆ alkyl, C₂-C₆ alkenyl or C₂-C₆ alkynyl;

X^{1a} is —NR^{11a}—, ═N—, —N═, —C(R^{11a})═ or ═C(R^{11a})—;

X^{2a} is —NR^{11a′}— or ═N—;

X^{3a} is —NR^{11a″}—, —N═ or —C(R^{11a′})═;

X^{4a} is —N═ or —C═;

X^{5a} is —NR^{12a}— or —CR^{12a}R^{12a′}—;

Y^{1a} is H, D, —OR^{13a}, —SR^{13a} or —NR^{13a}R^{13a′} when X^{1a} is —N═ or —C(R^{11a})═, or Y^{1a} is ═NR^{13a} when X^{1a} is —NR^{11a}—, ═N— or ═C(R^{11a})—;

Y^{2a} is H, D, C₁-C₆ alkyl, C₂-C₆ alkenyl, —C(O)R^{14a}, —C(O)OR^{14a} or —C(O)NR^{14a}R^{14a′} when X^{4a} is —C═, or Y^{2a} is absent when X^{4a} is —N═;

R^{1a′}, R^{2a′}, R^{3a′}, R^{11a}, R^{11a′}, R^{11a″}, R^{12a}, R^{12a′}, R^{13a}, R^{13a′}, R^{14a} and R^{14a′} are each independently selected from the group consisting of H, D, C₁-C₆ alkyl, C₂-C₆ alkenyl, C₂-C₆ alkynyl, —C(O)R^{15a}, —C(O)OR^{15a} and —C(O)NR^{15a}R^{15a′};

R^{4a′} and R^{5a′} are each independently selected from the group consisting of C₁-C₆ alkyl, C₂-C₆ alkenyl, C₂-C₆ alkynyl, —OR^{16a}, —SR^{16a}, —NR^{16a}R^{16a′}, provided that one of R^{4a′} and R^{5a′} is a covalent bond to an AA, a L¹ or a L²;

R^{15a}, R^{15a′}, R^{16a} and R^{16a′} are each independently H or C₁-C₆ alkyl;

m¹ is 1, 2, 3 or 4; and

each * is a covalent bond;

or a pharmaceutically acceptable salt thereof.

In some embodiments, the disclosure provides a conjugate selected from the group consisting of

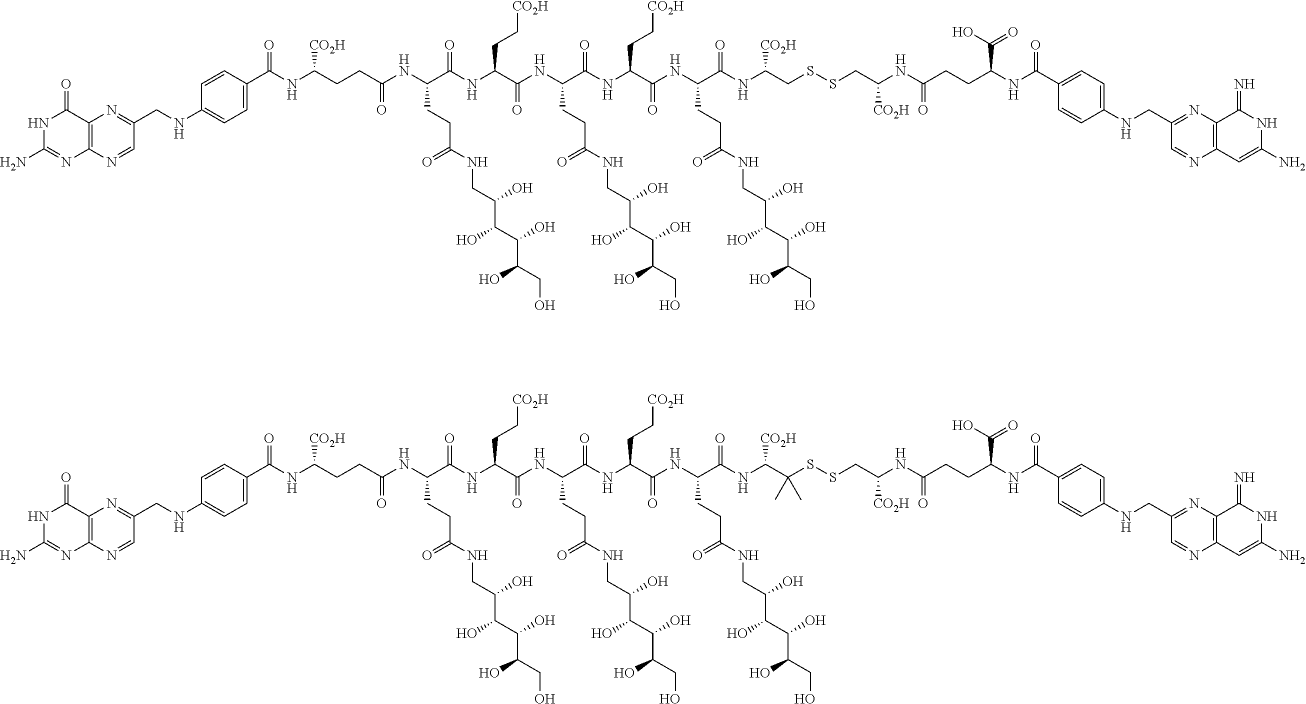

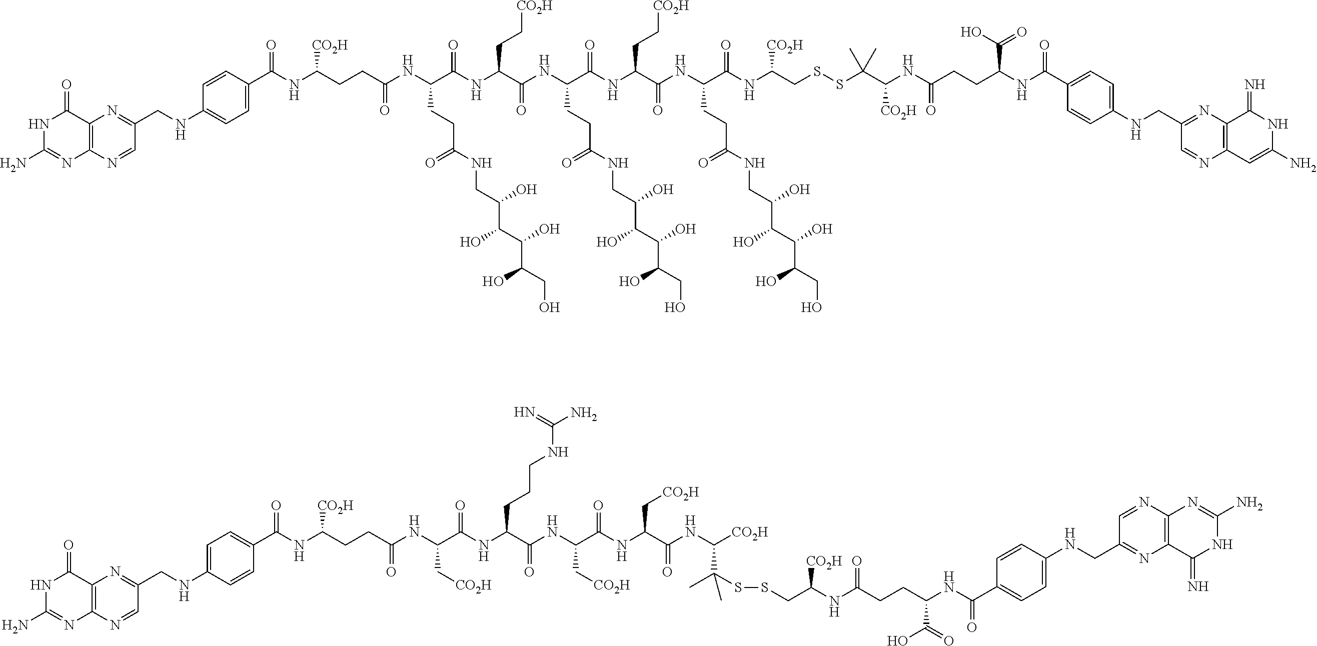

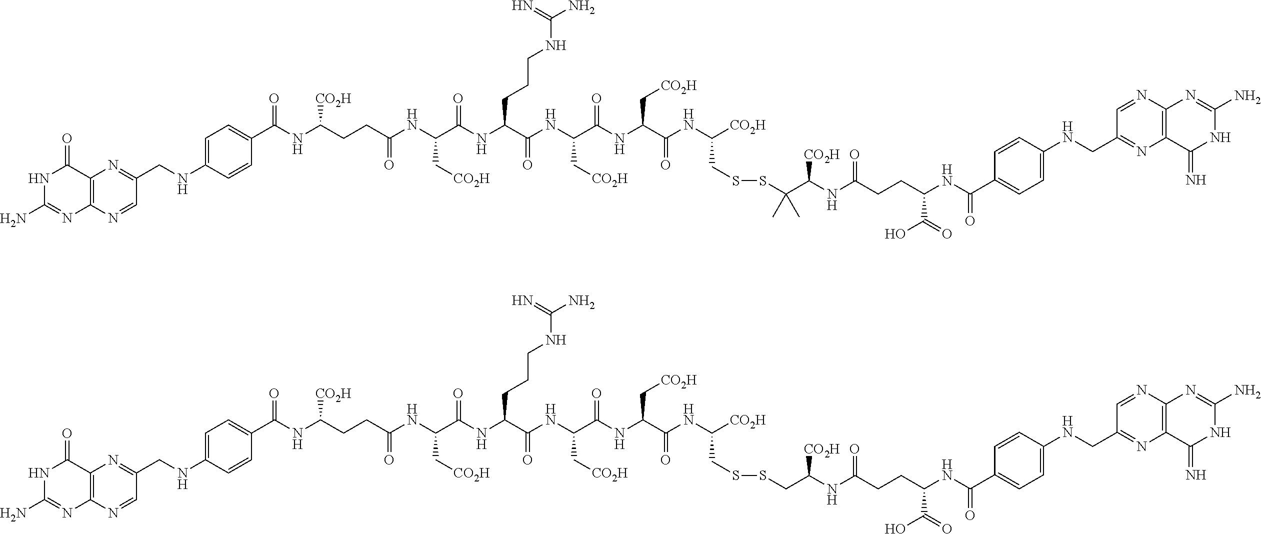 [SMILES: CC(C)(SSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c1ccc(NCc2cnc3nc(N)[nH]c(=O)c3n2)cc1)C(=O)O)C(=O)O)[C@H](NC(=O)CC[C@H](NC(=O)c1ccc(NCc2cnc3nc(N)[nH]c(=N)c3n2)cc1)C(=O)O)C(=O)O ; N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CSSC[C@H](NC(=O)CC[C@H](NC(=O)c1ccc(NCc2cnc3nc(N)[nH]c(=N)c3n2)cc1)C(=O)O)C(=O)O)C(=O)O]

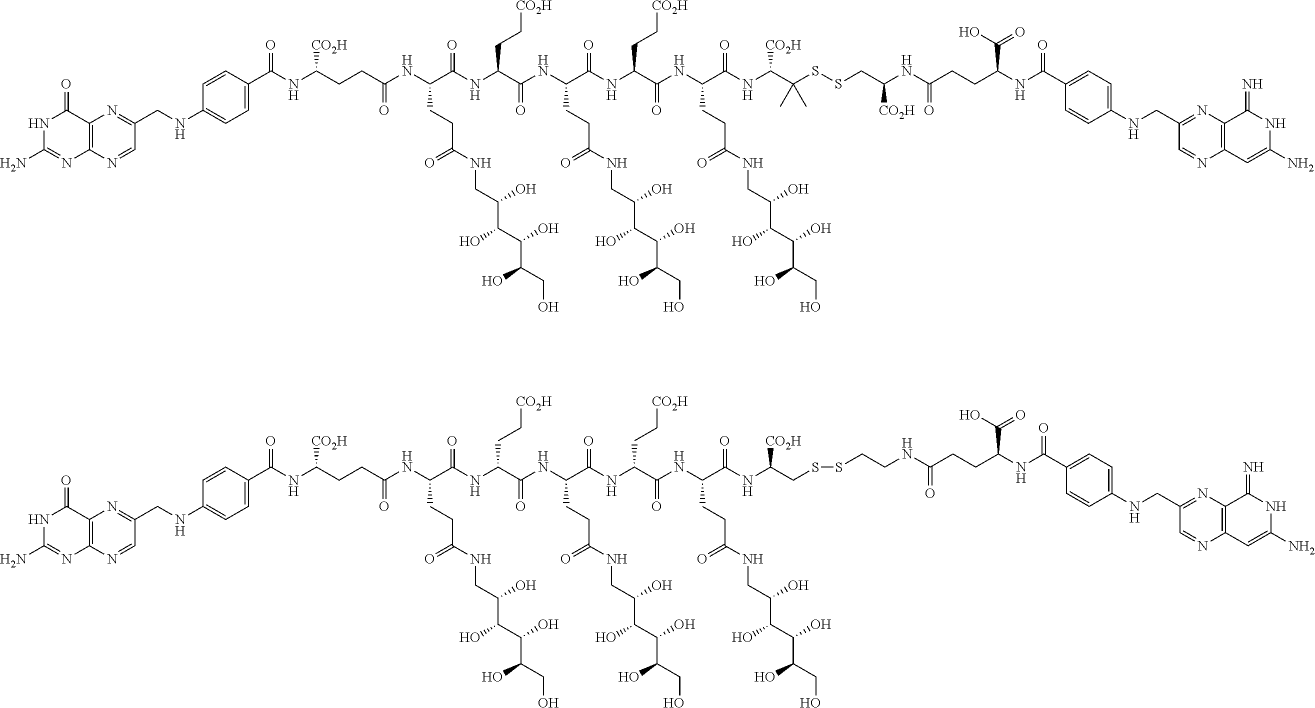

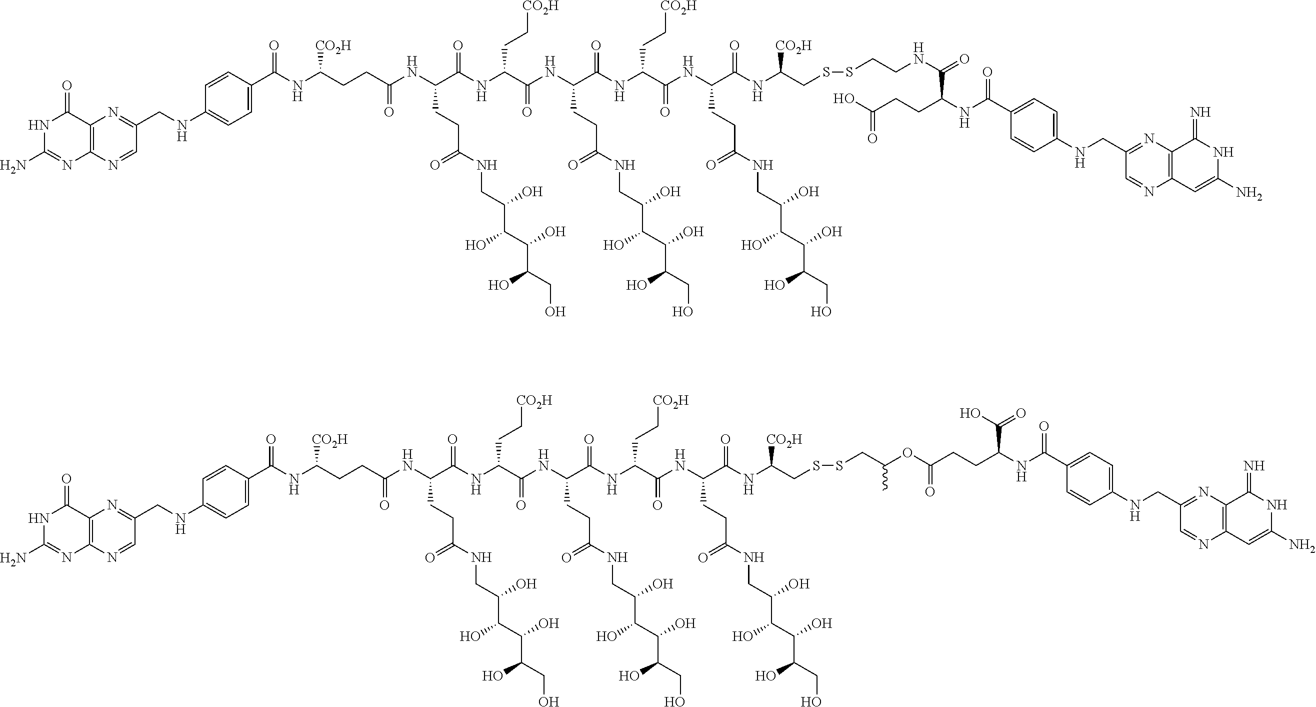

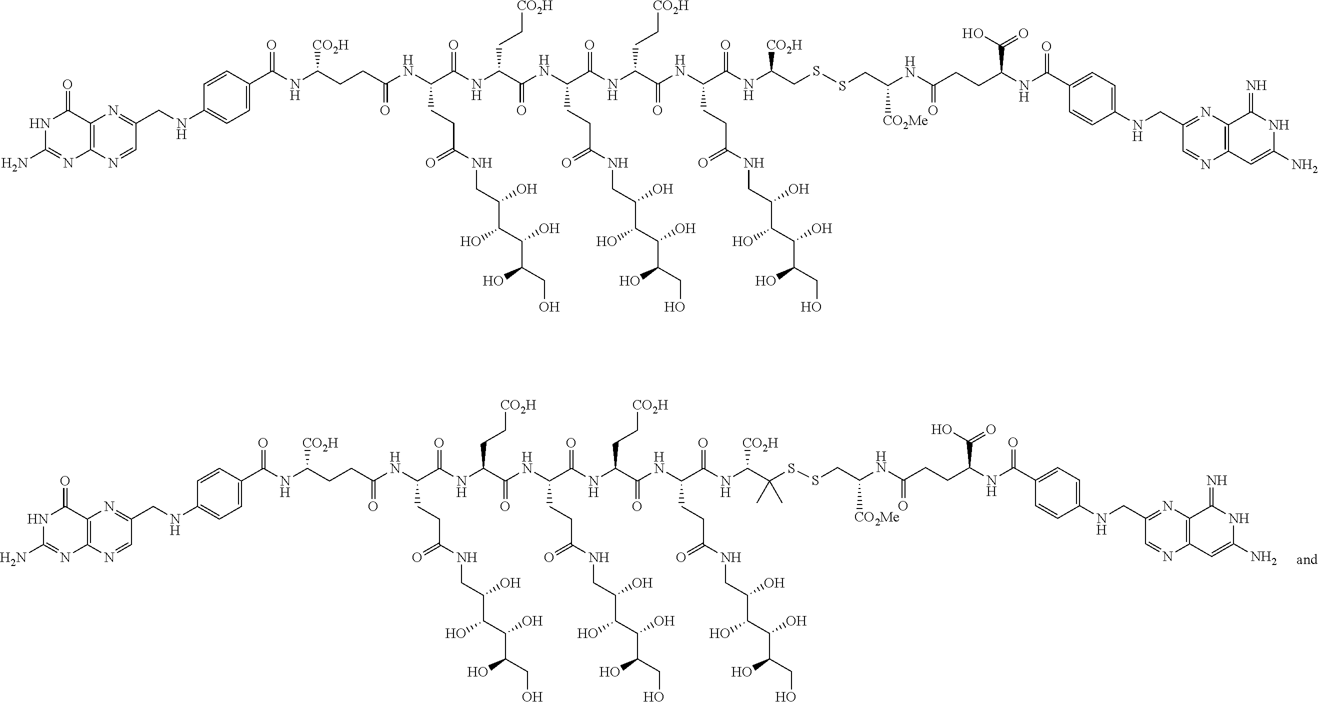

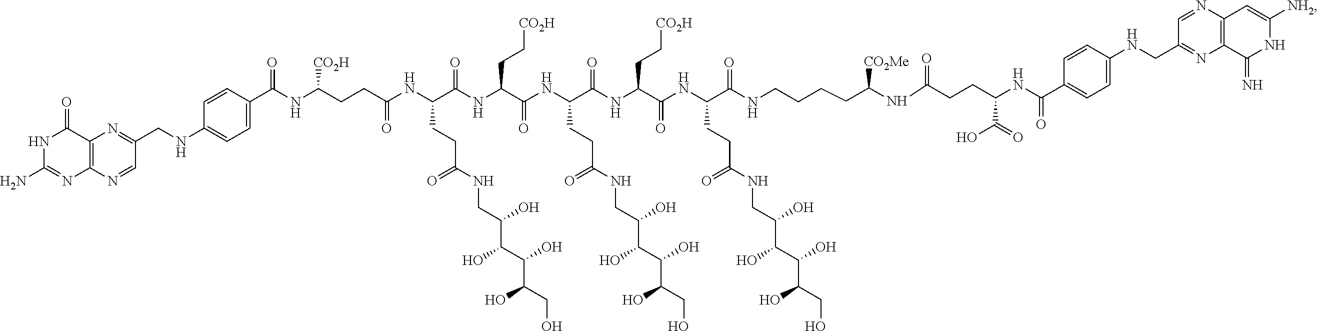 [SMILES: COC(=O)[C@H](CCCCNC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CC[C@H](NC(=O)c1ccc(NCc2cnc3nc(N)[nH]c(=O)c3n2)cc1)C(=O)O)NC(=O)CC[C@H](NC(=O)c1ccc(NCc2cnc3cc(N)[nH]c(=N)c3n2)cc1)C(=O)O]

or a pharmaceutically acceptable salt thereof.

In another aspect, the disclosure provides a pharmaceutical composition comprising a conjugate as described herein, or a pharmaceutically acceptable salt thereof, and at least one excipient. In one embodiment, a conjugate as described herein, or a pharmaceutically acceptable salt thereof, is included in an amount effective to treat disease states caused by pathogenic populations of cells, such as inflammatory cells.

In another aspect, the disclosure provides methods for treating diseases and disease states caused by pathogenic populations of cells, such as inflammatory cells comprising administering a therapeutically effective amount of a conjugate as described herein to a patient in need of such treatment.

In another aspect, the disclosure provides for the use of a conjugate as described herein in the preparation of a medicament for the treatment of inflammation.

In another aspect, the disclosure provides for the use of a conjugate as described herein for the treatment of inflammation.

The conjugates of the present disclosure can be described as embodiments in any of the following enumerated clauses. It will be understood that any of the embodiments described herein can be used in connection with any other embodiments described herein to the extent that the embodiments do not contradict one another.

1. A conjugate of the formula B-L-D¹, wherein B is a binding ligand of the formula

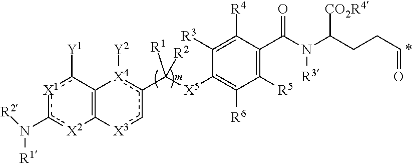

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;

X¹ is —NR¹¹—, ═N—, —N═, —C(R¹¹)═ or ═C(R¹¹)—;

X² is —NR^{11′}— or ═N—;

X³ is —NR^{11″}—, —N═ or —C(R^{11′})═;

X⁴ is —N═ or —C═;

X⁵ is NR¹² or CR¹²R^{12′};

Y¹ is H, D, —OR¹³ or —SR¹³ when X¹ is —N═ or —C(R¹¹)═, or Y¹ is ═O when X¹ is —NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;

R^{1′}, R^{2′}, R^{3′}, R^{4′}, R¹¹, R^{11′}, R^{11″}, R¹², R^{12′}, R¹³, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

L is a linker comprising at least one AA, at least one L¹ and an L², wherein each AA is an amino acid, each L¹ is of the formula

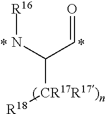

wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R²⁹;

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

and L² is of the formula

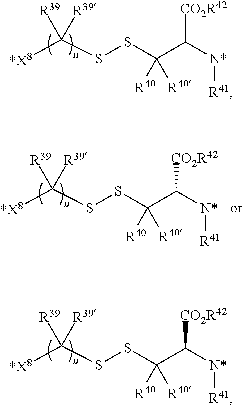

wherein

X⁸ is —NR⁵⁰— or —O—;

each R³⁹, R^{39′}, R⁴⁰ and R^{40′} is independently selected from the group consisting of H, D, C₁-C₆ alkyl, C₂-C₆ alkenyl, C₂-C₆ alkynyl C₃-C₆ cycloalkyl, —OR⁴⁸, —OC(O)R⁴⁸, —OC(O)NR⁴⁸R^{48′}, —OS(O)R⁴⁸, —OS(O)₂R⁴⁸, —SR⁴⁸, —S(O)R⁴⁸, —S(O)₂R⁴⁸, —S(O)NR⁴⁸R^{48′}, —S(O)₂NR⁴⁸R^{48′}, —OS(O)NR⁴⁸R^{48′}, —OS(O)₂NR⁴⁸R^{48′}, —NR⁴⁸R^{48′}, —NR⁴⁸C(O)R⁴⁹, —NR⁴⁸C(O)OR⁴⁹, —NR⁴⁸C(O)NR⁴⁹R^{49′}, —NR⁴⁸S(O)R⁴⁹, —NR⁴⁸S(O)₂R⁴⁹, —NR⁴⁸S(O)NR⁴⁹R^{49′}, —NR⁴⁸S(O)₂NR⁴⁹R^{49′}, —C(O)R⁴⁸, —C(O)OR⁴⁸ or —C(O)NR⁴⁸R^{48′}, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR⁴⁴, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};

each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};

each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′};

each R⁴³, R^{43′}, R⁴⁴, R^{44′}, R⁴⁵, R^{45′}, R⁴⁶, R^{46′}, R⁴⁷, R^{47′}, R⁴⁸, R^{48′}, R⁴⁹, R^{49′} and R⁵⁰ is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl; and

u is 1, 2, 3 or 4; and

D¹ is a drug of the formula

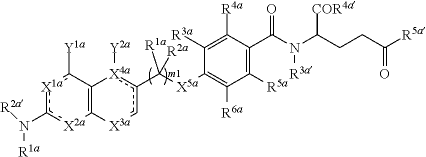

wherein

R^{1a} and R^{2a} in each instance are independently selected from the group consisting of H, D, halogen, C₁-C₆ alkyl, C₂-C₆ alkenyl, C₂-C₆ alkynyl, —OR^{7a}, —SR^{7a} and —NR^{7a}R^{7a′}, wherein each hydrogen atom in C₁-C₆ alkyl, C₂-C₆ alkenyl and C₂-C₆ alkynyl is independently optionally substituted by halogen, —OR^{8a}, —SR^{8a}, —NR^{8a}R^{8a′}, —C(O)R^{8a}, —C(O)OR^{8a} or —C(O)NR^{8a}R^{8a′};

R^{3a}, R^{4a}, R^{5a} and R^{6a} are each independently selected from the group consisting of H, D, halogen, C₁-C₆ alkyl, C₂-C₆ alkenyl, C₂-C₆ alkynyl, —CN, —NO₂, —NCO, —OR^{9a}, —SR^{9a}, —NR^{9a}R^{9a′}, —C(O)R^{9a}, —C(O)OR^{9a} and —C(O)NR^{9a}R^{9a′}, wherein each hydrogen atom in C₁-C₆ alkyl, C₂-C₆ alkenyl and C₂-C₆ alkynyl is independently optionally substituted by halogen, —OR^{10a}, —SR^{10a}, —NR^{10a}R^{10a′}, —C(O)R^{10a}, —C(O)OR^{10a} or —C(O)NR^{10a}R^{10a′};

each R^{7a}, R^{7a′}, R^{8a}, R^{8a′}, R^{9a}, R^{9a′}, R^{10a} and R^{10a′} is independently H, D, C₁-C₆ alkyl, C₂-C₆ alkenyl or C₂-C₆ alkynyl;

X^{1a} is —NR^{11a}—, ═N—, —N═, —C(R^{11a})═ or ═C(R^{11a})—;

X^{2a} is —NR^{11a′}— or ═N—;

X^{3a} is —NR^{11a″}—, —N═ or —C(R^{11a′})═;

X^{4a} is —N═ or —C═;

X^{5a} is —NR^{12a}— or —CR^{12a}R^{12a′}—;

Y^{1a} is —NR^{13a}R^{13a′} when X^{1a} is —N═ or —C(R^{11a})═, or Y^{1a} is ═NR^{13a} when X^{1a} is —NR^{11a}—, ═N— or ═C(R^{11a})—;

Y^{2a} is H, D, C₁-C₆ alkyl, C₂-C₆ alkenyl, —C(O)R^{14a}, —C(O)OR^{14a} or —C(O)NR^{14a}R^{14a′} when X^{4a} is —C═, or Y^{2a} is absent when X^{4a} is —N═;

R^{1a′}, R^{2a′}, R^{3a′}, R^{11a}, R^{11a′}, R^{11a″}, R^{12a}, R^{12a′}, R^{13a}, R^{13a′}, R^{14a} and R^{14a′} are each independently selected from the group consisting of H, D, C₁-C₆ alkyl, C₂-C₆ alkenyl, C₂-C₆ alkynyl, —C(O)R^{15a}, —C(O)OR^{15a} and —C(O)NR^{15a}R^{15a′};

R^{4a′} and R^{5a′} are each independently selected from the group consisting of C₁-C₆ alkyl, C₂-C₆ alkenyl, C₂-C₆ alkynyl, —OR^{16a}, —SR^{16a}, —NR^{16a}R^{16a′}, provided that one of R^{4a′} and R^{5a′} is a covalent bond to an AA, a L¹ or a L²;

R^{15a}, R^{15a′}, R^{16a} and R^{16a′} are each independently H or C₁-C₆ alkyl;

m¹ is 1, 2, 3 or 4; and

each * is a covalent bond;

or a pharmaceutically acceptable salt thereof.
2. A conjugate of the formula B-L-D¹, wherein B is a binding ligand of the formula

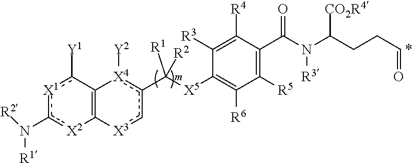

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;

X¹ is —NR¹¹—, ═N—, —N═, —C(R¹¹)═ or ═C(R¹¹)—;

X² is —NR^{11′}— or ═N—;

X³ is —NR^{11″}—, —N═ or —C(R^{11′})═;

X⁴ is —N═ or —C═;

X⁵ is NR¹² or CR¹²R^{12′};

Y¹ is H, D, —OR¹³ or —SR¹³ when X¹ is —N═ or —C(R¹¹)═, or Y¹ is ═O when X¹ is —NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;

R^{1′}, R^{2′}, R^{3′}, R^{4′}, R¹¹, R^{11′}, R^{11″}, R¹², R^{12′}, R¹³, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

L is a linker comprising at least one AA, at least one L¹ and an L², wherein each AA is an amino acid, each L¹ is of the formula

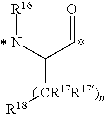

wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5; and

q is 1, 2, 3, 4 or 5;

and L² is of the formula

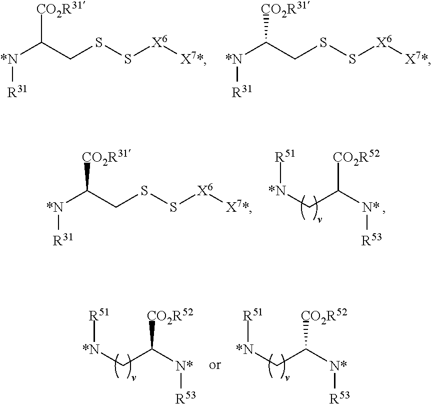

wherein

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};

each X⁷ is —NR^{31a}— or —O—, and when X⁶ is C₁-C₆ alkyl and X⁷ is —O—, then at least one hydrogen atom in C₁-C₆ alkyl is substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};

each R³¹ and R^{31a} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

each R⁵¹ and R⁵³ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁴, —OC(O)R⁵⁴, —OC(O)NR⁵⁴R^{54′}, —OS(O)R⁵⁴, —OS(O)₂R⁵⁴, —SR⁵⁴, —S(O)R⁵⁴, —S(O)₂R⁵⁴, —S(O)NR⁵⁴R^{54′}, —S(O)₂NR⁵⁴R^{54′}, —OS(O)NR⁵⁴R^{54′}, —OS(O)₂NR⁵⁴R^{54′}, —NR⁵⁴R^{54′}, —NR⁵⁴C(O)R⁵⁵, —NR⁵⁴C(O)OR⁵⁵, —NR⁵⁴C(O)NR⁵⁵R^{55′}, —NR⁵⁴S(O)R⁵⁵, —NR⁵⁴S(O)₂R⁵⁵, —NR⁵⁴S(O)NR⁵⁵R^{55′}, —NR⁵⁴S(O)₂NR⁵⁵R^{55′}, —C(O)R⁵⁴, —C(O)OR⁵⁴ or —C(O)NR⁵⁴R^{54′};

each R⁵² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁵⁶, —OC(O)R⁵⁶, —OC(O)NR⁵⁶R^{56′}, —OS(O)R⁵⁶, —OS(O)₂R⁵⁶, —SR⁵⁶, —S(O)R⁵⁶, —S(O)₂R⁵⁶, —S(O)NR⁵⁶R^{56′}, —S(O)₂NR⁵⁶R^{56′}, —OS(O)NR⁵⁶R^{56′}, —OS(O)₂NR⁵⁶R^{56′}, —NR⁵⁶R^{56′}, —C(O)R⁵⁶, —C(O)OR⁵⁶ or —C(O)NR⁵⁶R^{56′};

each R⁵⁴, R^{54′}, R⁵⁵, R^{55′}, R⁵⁶ and R^{56′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl; and

v is 1, 2, 3, 4, 5 or 6; and

D¹ is a drug of the formula I

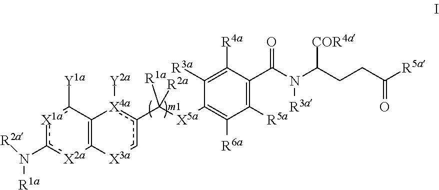

wherein

R^{1a} and R^{2a} in each instance are independently selected from the group consisting of H, D, halogen, C₁-C₆ alkyl, C₂-C₆ alkenyl, C₂-C₆ alkynyl, —OR^{7a}, —SR^{7a} and —NR^{7a}R^{7a′}, wherein each hydrogen atom in C₁-C₆ alkyl, C₂-C₆ alkenyl and C₂-C₆ alkynyl is independently optionally substituted by halogen, —OR^{8a}, —SR^{8a}, —NR^{8a}R^{8a′}, —C(O)R^{8a}, —C(O)OR^{8a} or —C(O)NR^{8a}R^{8a′};

R^{3a}, R^{4a}, R^{5a} and R^{6a} are each independently selected from the group consisting of H, D, halogen, C₁-C₆ alkyl, C₂-C₆ alkenyl, C₂-C₆ alkynyl, —CN, —NO₂, —NCO, —OR^{9a}, —SR^{9a}, —NR^{9a}R^{9a′}, —C(O)R^{9a}, —C(O)OR^{9a} and —C(O)NR^{9a}R^{9a′}, wherein each hydrogen atom in C₁-C₆ alkyl, C₂-C₆ alkenyl and C₂-C₆ alkynyl is independently optionally substituted by halogen, —OR^{10a}, —SR^{10a}, —NR^{10a}R^{10a′}, —C(O)R^{10a}, —C(O)OR^{10a} or —C(O)NR^{10a}R^{10a′};

each R^{7a}, R^{7a′}, R^{8a}, R^{8a′}, R^{9a}, R^{9a′}, R^{10a} and R^{10a′} is independently H, D, C₁-C₆ alkyl, C₂-C₆ alkenyl or C₂-C₆ alkynyl;

X^{1a} is —NR^{11a}—, ═N—, —N═, —C(R^{11a})═ or ═C(R^{11a})—;

X^{2a} is —NR^{11a′}— or ═N—;

X^{3a} is —NR^{11a″}—, —N═ or —C(R^{11a′})═;

X^{4a} is —N═ or —C═;

X^{5a} is —NR^{12a}— or —CR^{12a}R^{12a′}—;

Y^{1a} is H, D, —OR^{13a}, —SR^{13a} or —NR^{13a}R^{13a′} when X^{1a} is —N═ or —C(R^{11a})═, or Y^{1a} is ═NR^{13a} when X^{1a} is —NR^{11a}—, ═N— or ═C(R^{11a})—;

Y^{2a} is H, D, C₁-C₆ alkyl, C₂-C₆ alkenyl, —C(O)R^{14a}, —C(O)OR^{14a} or —C(O)NR^{14a}R^{14a′} when X^{4a} is —C═, or Y^{2a} is absent when X^{4a} is —N═;

R^{1a′}, R^{2a′}, R^{3a′}, R^{11a}, R^{11a′}, R^{11a″}, R^{12a}, R^{12a′}, R^{13a}, R^{13a′}, R^{14a} and R^{14a′} are each independently selected from the group consisting of H, D, C₁-C₆ alkyl, C₂-C₆ alkenyl, C₂-C₆ alkynyl, —C(O)R^{15a}, —C(O)OR^{15a} and —C(O)NR^{15a}R^{15a′};

R^{4a′} and R^{5a′} are each independently selected from the group consisting of C₁-C₆ alkyl, C₂-C₆ alkenyl, C₂-C₆ alkynyl, —OR^{16a}, —SR^{16a}, —NR^{16a}R^{16a′}, provided that one of R^{4a′} and R^{5a′} is a covalent bond to an AA, a L¹ or a L²;

R^{15a}, R^{15a′}, R^{16a} and R^{16a′} are each independently H or C₁-C₆ alkyl;

m¹ is 1, 2, 3 or 4; and

each * is a covalent bond;

or a pharmaceutically acceptable salt thereof.
3. The conjugate of clause 1 or 2, having the formula B-L¹-AA-L¹-AA-L¹-L²-D¹, B-AA-L¹-AA-AA-L²-D¹, or B-AA-AA-AA-AA-L²-D¹, or a pharmaceutically acceptable salt thereof.
4. The conjugate of clauses 1 to 3, or a pharmaceutically acceptable salt thereof, wherein m is 1.
5. The conjugate of clauses 1 or 4, or a pharmaceutically acceptable salt thereof, wherein X¹ is —NR¹¹—.
6. The conjugate of any one of clauses 1 to 5, or a pharmaceutically acceptable salt thereof, wherein X² is ═N—.
7. The conjugate of any one of clauses 1 to 6, or a pharmaceutically acceptable salt thereof, wherein Y¹ is ═O.
8. The conjugate of any one of clauses 1 to 7, or a pharmaceutically acceptable salt thereof, wherein X¹ is —NR¹¹—, and R¹¹ is H.
9. The conjugate of any one of clauses 1 to 8, or a pharmaceutically acceptable salt thereof, wherein X³ is —C(R¹¹)═.
10. The conjugate of clause 9, or a pharmaceutically acceptable salt thereof, wherein R¹¹ is H.
11. The conjugate of any one of clauses 1 to 10, or a pharmaceutically acceptable salt thereof, wherein X⁴ is —C═.
12. The conjugate of any one of clauses 1 to 11, or a pharmaceutically acceptable salt thereof, wherein Y² is H.
13. The conjugate of any one of clauses 1 to 8, or a pharmaceutically acceptable salt thereof, wherein X³ is —N═.
14. The conjugate of any one of clauses 1 to 8 or 13, or a pharmaceutically acceptable salt thereof, wherein X⁴ is —N═.
15. The conjugate of any one of clauses 1 to 14, or a pharmaceutically acceptable salt thereof, wherein X⁵ is —NR¹²—.
16. The conjugate of any one of clauses 1 to 15, or a pharmaceutically acceptable salt thereof, wherein R¹² is H.
17. The conjugate of any one of clauses 1 to 16, or a pharmaceutically acceptable salt thereof, wherein R^{1′} and R^{2′} are H.
18. The conjugate of any one of clauses 1 to 17, or a pharmaceutically acceptable salt thereof, wherein R^{3′} is H.
19. The conjugate of any one of clauses 1 to 18, or a pharmaceutically acceptable salt thereof, wherein R^{4′} is H.
20. The conjugate of any one of clauses 1 to 19, or a pharmaceutically acceptable salt thereof, wherein each R¹ and R² is H.
21. The conjugate of any one of clauses 1 to 20, or a pharmaceutically acceptable salt thereof, wherein R³, R⁴, R⁵ and R⁶ are H.
22. The conjugate of any one of clauses 1 to 21, or a pharmaceutically acceptable salt thereof, wherein m¹ is 1.
23. The conjugate of any one of clauses 1 to 22, or a pharmaceutically acceptable salt thereof, wherein X^{1a} is —NR^{11a}—.
24. The conjugate of any one of clauses 1 to 23, or a pharmaceutically acceptable salt thereof, wherein X^{2a} is ═N—.
25. The conjugate of any one of clauses 1 to 24, or a pharmaceutically acceptable salt thereof, wherein Y^{1a} is ═NR^{13a}.
26. The conjugate of any one of clauses 1 to 25, or a pharmaceutically acceptable salt thereof, wherein X^{1a} is —NR^{11a}—, and R^{11a} is H.
27. The conjugate of any one of clauses 1 to 26, or a pharmaceutically acceptable salt thereof, wherein X^{3a} is —C(R^{11a′})═.
28. The conjugate of clause 27, or a pharmaceutically acceptable salt thereof, wherein R^{11a′} is H.
29. The conjugate of any one of clauses 1 to 28, or a pharmaceutically acceptable salt thereof, wherein X^{4a} is —C═.
30. The conjugate of any one of clauses 1 to 29, or a pharmaceutically acceptable salt thereof, wherein Y^{2a} is H.
31. The conjugate of any one of clauses 1 to 26, or a pharmaceutically acceptable salt thereof, wherein X^{3a} is —N═.
32. The conjugate of any one of clauses 1 to 26 or 31, or a pharmaceutically acceptable salt thereof, wherein X^{4a} is —N═.
33. The conjugate of any one of clauses 1 to 32, or a pharmaceutically acceptable salt thereof, wherein X^{5a} is —NR^{12a}—.
34. The conjugate of any one of clauses 1 to 33, or a pharmaceutically acceptable salt thereof, wherein R^{12a} is H.
35. The conjugate of any one of clauses 1 to 34, or a pharmaceutically acceptable salt thereof, wherein R^{1a′} and R^{2a′} are H.
36. The conjugate of any one of clauses 1 to 35, or a pharmaceutically acceptable salt thereof, wherein R^{3a′} is H.
37. The conjugate of any one of clauses 1 to 36, or a pharmaceutically acceptable salt thereof, wherein R^{4a′} is H.
38. The conjugate of any one of clauses 1 to 37, or a pharmaceutically acceptable salt thereof, wherein each R^{1a} and R^{2a} is H.
39. The conjugate of any one of clauses 1 to 38, or a pharmaceutically acceptable salt thereof, wherein R^{3a}, R^{4a}, R^{5a} and R^{6a} are H.
40. The conjugate of any one of clauses 1 to 39, or a pharmaceutically acceptable salt thereof, wherein X⁸ is —NR⁵⁰—.
41. The conjugate of clause 40, or a pharmaceutically acceptable salt thereof, wherein R⁵⁰ is H.
42. The conjugate of any one of clauses 1 to 39, or a pharmaceutically acceptable salt thereof, wherein X⁸ is —O—.
43. The conjugate of any one of clauses 1 to 42, or a pharmaceutically acceptable salt thereof, wherein u is 2.
44. The conjugate of any one of clauses 1 to 43, or a pharmaceutically acceptable salt thereof, wherein R⁴² is C₁-C₆ alkyl.
45. The conjugate of any one of clauses 1 to 43, or a pharmaceutically acceptable salt thereof, wherein R⁴² is H.
46. The conjugate of any one of clauses 1 to 45, or a pharmaceutically acceptable salt thereof, wherein R⁴¹ is H.
47. The conjugate of any one of clauses 1 to 46, or a pharmaceutically acceptable salt thereof, wherein R⁴⁰ and R^{40′} are selected from H, C₁-C₆ alkyl and —C(O)OR⁴⁸.
48. The conjugate of any one of clauses 1 to 47, or a pharmaceutically acceptable salt thereof, wherein R⁴⁰ and R^{40′} are C₁-C₆ alkyl.
49. The conjugate of clause 48, wherein R⁴⁰ and R^{40′} are methyl.
50. The conjugate of any one of clauses 1 to 47, or a pharmaceutically acceptable salt thereof, wherein R⁴⁰ and R^{40′} are H.
51. The conjugate of clause 50, or a pharmaceutically acceptable salt thereof, wherein R⁴⁸ is H.
52. The conjugate of any one of clauses 1 or 2 to 39, or a pharmaceutically acceptable salt thereof, wherein L² is of a formula selected from

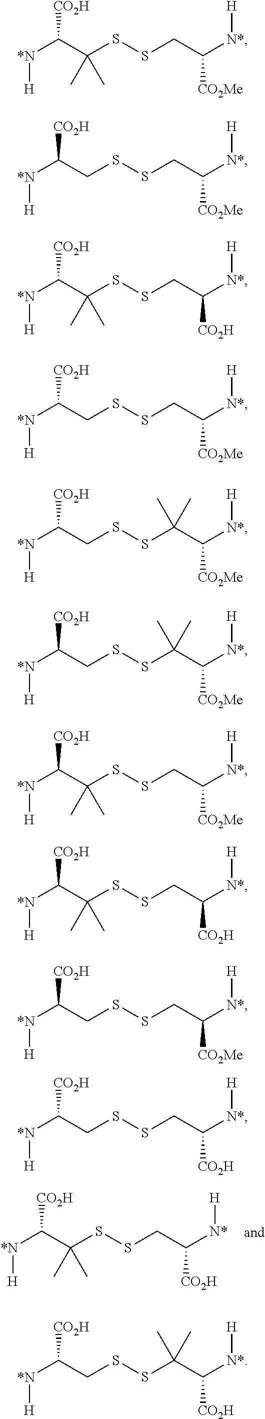

53. The conjugate of clause 52, or a pharmaceutically acceptable salt thereof, wherein L² is of the formula

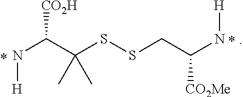 [SMILES: COC(=O)[C@@H](N)CSSC(C)(C)[C@H](N)C(=O)O]

54. The conjugate of any one of clauses 2 to 39, or a pharmaceutically acceptable salt thereof, wherein X⁶ is C₁-C₆ alkyl, and each hydrogen atom in C₁-C₆ alkyl is optionally substituted by a C₁-C₆ alkyl.
55. The conjugate of any one of clauses 2 to 39 or 54, or a pharmaceutically acceptable salt thereof, wherein X⁷ is —NR^{31a}—.
56. The conjugate of clause 55, or a pharmaceutically acceptable salt thereof, wherein R^{31a} is H.
57. The conjugate of any one of clauses 2 to 39 or 54 to 56, or a pharmaceutically acceptable salt thereof, wherein X⁷ is —O—.
58. The conjugate of any one of clauses 2 to 39 or 54 to 57, or a pharmaceutically acceptable salt thereof, wherein R³¹ is H.
59. The conjugate of any one of clauses 2 to 39 or 54 to 58, or a pharmaceutically acceptable salt thereof, R³¹ is H.
60. The conjugate of any one of clauses 2 to 39, or a pharmaceutically acceptable salt thereof, wherein v is 4.
61. The conjugate of any one of clauses 2 to 39 or 60, or a pharmaceutically acceptable salt thereof, wherein R⁵¹ is H.
62. The conjugate of any one of clauses 2 to 39, 60 or 61, or a pharmaceutically acceptable salt thereof, wherein R⁵² is C₁-C₆ alkyl.
63. The conjugate of clause 62, or a pharmaceutically acceptable salt thereof, wherein R⁵² is methyl.
64. The conjugate of any one of clauses 2 to 39 or 60 to 63, or a pharmaceutically acceptable salt thereof, wherein R⁵³ is H.
65. The conjugate of any one of clauses 1 to 64, or a pharmaceutically acceptable salt thereof, wherein at least one AA is in the D-configuration.
66. The conjugate of any one of clauses 1 to 64, or a pharmaceutically acceptable salt thereof, wherein at least two AA are in the D-configuration.
67. The conjugate of any one of clauses 1 to 66, or a pharmaceutically acceptable salt thereof, wherein AA is selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline.
68. The conjugate of any one of clauses 1 to 67, or a pharmaceutically acceptable salt thereof, wherein AA is selected from the group consisting of L-arginine, D-arginine, L-aspartic acid, D-aspartic acid, L-glutamic acid and D-glutamic acid.
69. The conjugate of clause 1, selected from the group consisting of

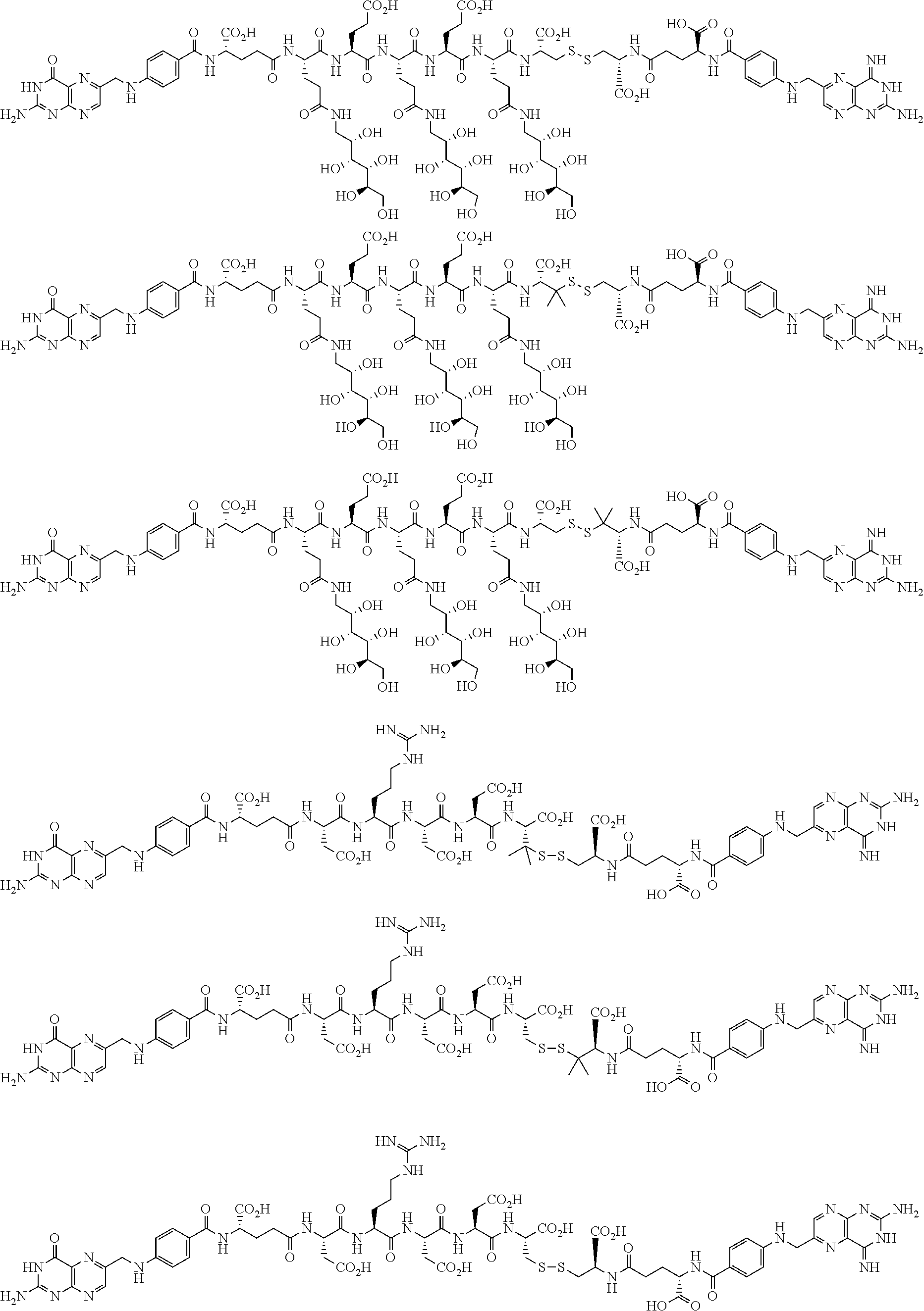

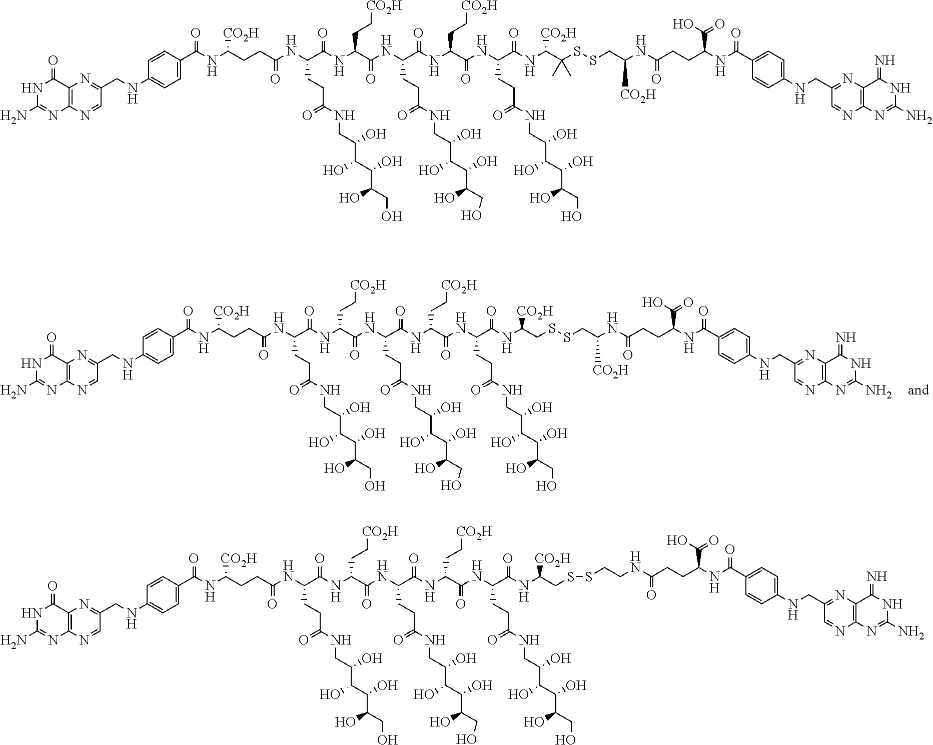

or a pharmaceutically acceptable salt thereof.
70. The conjugate of clause 2, selected from the group consisting of

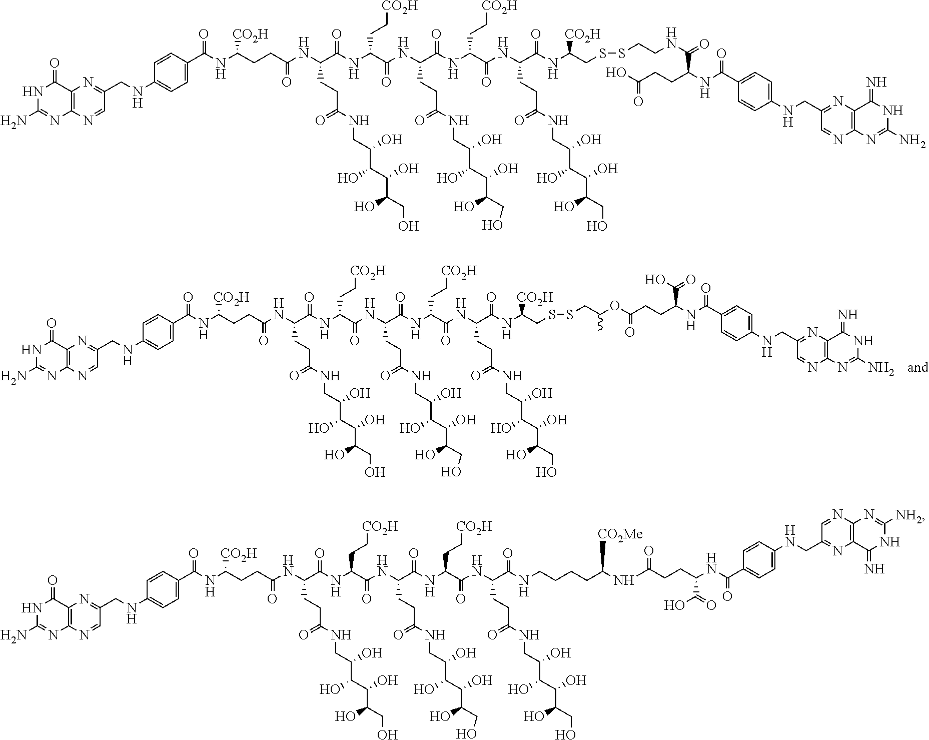

or a pharmaceutically acceptable salt thereof.
71. A pharmaceutical composition comprising a conjugate of any one of clauses 1 to 70, or a pharmaceutically acceptable salt thereof, and optionally at least one excipient.
72. The pharmaceutical composition of clause 71, wherein the conjugate, or a pharmaceutically acceptable salt thereof, is included in an amount effective to treat disease states caused by inflammatory cells.
73. A method for treating diseases and disease states caused by inflammation comprising administering a therapeutically effective amount of a conjugate of any one of clauses 1 to 70, or a pharmaceutically acceptable salt thereof, to a patient in need of such treatment.
74. The method of clause 73, wherein the disease caused by inflammation is selected from the group consisting of arthritis, rheumatoid arthritis, osteoarthritis, glomerulonephritis, proliferative retinopathy, restenosis, ulcerative colitis, Crohn's disease, fibromyalgia, psoriasis and other inflammations of the skin, inflammations of the eye, including uveitis and autoimmune uveitis, osteomyelitis, Sögren's syndrome, multiple sclerosis, diabetes, atherosclerosis, pulmonary fibrosis, lupus erythematosus, sarcoidosis, systemic sclerosis, organ transplant rejection (GVHD) and chronic inflammations.
75. Use of a conjugate according to any one of clauses 1 to 70, or a pharmaceutically acceptable salt thereof, in the preparation of a medicament for the treatment of inflammation.
76. Use of a conjugate according to any one of clauses 1 to 70, or a pharmaceutically acceptable salt thereof, for the treatment of inflammation.
77. Use of a conjugate according to any one of clauses 1 to 70, or a pharmaceutically acceptable salt thereof, in the preparation of a medicament for the treatment of a disease or disease state caused by inflammatory cells.
78. The use of clause 77, wherein the disease or disease state caused by inflammatory cells is selected from the group consisting of arthritis, rheumatoid arthritis, osteoarthritis, glomerulonephritis, proliferative retinopathy, restenosis, ulcerative colitis, Crohn's disease, fibromyalgia, psoriasis and other inflammations of the skin, inflammations of the eye, including uveitis and autoimmune uveitis, osteomyelitis, Sögren's syndrome, multiple sclerosis, diabetes, atherosclerosis, pulmonary fibrosis, lupus erythematosus, sarcoidosis, systemic sclerosis, organ transplant rejection (GVHD) and chronic inflammation.
79. Use of a conjugate according to any one of clauses 1 to 70, or a pharmaceutically acceptable salt thereof, for the treatment of a disease or disease state caused by inflammatory cells.
80. The use of clause 79, wherein disease or disease state caused by inflammatory cells is selected from the group consisting of arthritis, rheumatoid arthritis, osteoarthritis, glomerulonephritis, proliferative retinopathy, restenosis, ulcerative colitis, Crohn's disease, fibromyalgia, psoriasis and other inflammations of the skin, inflammations of the eye, including uveitis and autoimmune uveitis, osteomyelitis, Sögren's syndrome, multiple sclerosis, diabetes, atherosclerosis, pulmonary fibrosis, lupus erythematosus, sarcoidosis, systemic sclerosis, organ transplant rejection (GVHD) and chronic inflammation.

### BRIEF DESCRIPTION OF THE DRAWINGS

FIG. 1 shows the relative affinity of EC2319 was measured using KB cells. EC2319 displayed a high relative affinity value of 0.493 normalized against 1 for FA.

FIG. 2A shows that EC2319 was evaluated for its anti-proliferative activity against mouse RAW264.7 macrophage cells. As determined by the XTT assay, EC2319 showed a dose-dependent inhibition of cell proliferation with relative IC₅₀ values of ˜2.9 nM.

FIG. 2B shows that EC2319 was evaluated for its anti-proliferative activity against human THP-1-FRβ cells. As determined by the XTT assay, EC2319 showed a dose-dependent inhibition of cell proliferation with relative IC₅₀ values of ˜8.7 nM on THP-1-FRβ cells.

FIG. 3A shows a comparison of arthritic scores in rats treated according to the methods described herein; (●) control, (⋄) EC1669, (▴) EC2285, (▾) EC2318 and (♦) EC2319.

FIG. 3B shows a comparison of increased paw weight in rats treated according to the methods described herein between control, EC1669, EC2285, EC2318 and EC2319.

FIG. 3C shows a comparison of increased spleen weight in rats treated according to the methods described herein between control, EC1669, EC2285, EC2318 and EC2319.

FIG. 3D shows a comparison of body weight change in rats treated according to the methods described herein; (●) control, (⋄) EC1669, (▴) EC2285, (▾) EC2318 and (♦) EC2319.

FIG. 4A shows a comparison of arthritic scores in rats treated according to the methods described herein; (●) control, (◯) EC1669 (500 nmol/kg, BIW), (▴) EC2285 (500 nmol/kg, BIW), (Δ) EC2285 (500 nmol/kg, BIW)+500-fold excess EC0923, (♦) EC2319 (500 nmol/kg, BIW) and (⋄) EC2319 (500 nmol/kg, BIW)+500-fold excess EC0923.

FIG. 4B shows a comparison of increased paw weight in rats treated according to the methods described herein between control, EC1669, EC2285, EC2285+EC0923, EC2319 and EC2319+EC0923.

FIG. 4C shows a comparison of increased spleen weight in rats treated according to the methods described herein between control, EC1669, EC2285, EC2285+EC0923, EC2319 and EC2319+EC0923.

FIG. 4D shows a comparison of body weight change in rats treated according to the methods described herein; (●) control, (◯) EC1669 (500 nmol/kg, BIW), (▴) EC2285 (500 nmol/kg, BIW), (Δ) EC2285 (500 nmol/kg, BIW)+500-fold excess EC0923, (♦) EC2319 (500 nmol/kg, BIW) and (⋄) EC2319 (500 nmol/kg, BIW)+500-fold excess EC0923.

FIG. 5A shows a comparison of arthritic scores in rats treated according to the methods described herein; (●) control, (◯) EC2413 (1000 nmol/kg, SIW), (▴) EC2413 (500 nmol/kg, BIW), (Δ) EC2413 (500 nmol/kg, BIW)+500-fold excess EC0923, (□) EC1669 (500 nmol/kg, BIW) and (♦) EC2319 (500 nmol/kg, BIW).

FIG. 5B shows a comparison of increased paw weight in rats treated according to the methods described herein between control, EC2413 (1000 nmol/kg, SIW), EC2413 (500 nmol/kg, BIW), EC2413+EC0923, EC1669 and EC2319.

FIG. 5C shows a comparison of spleen weight in rats treated according to the methods described herein between control, EC2413 (1000 nmol/kg, SIW), EC2413 (500 nmol/kg, BIW), EC2413+EC0923, EC1669 and EC2319.

FIG. 5D shows a comparison of body weight change in rats treated according to the methods described herein; (●) control, (◯) EC2413 (1000 nmol/kg, SIW), (▴) EC2413 (500 nmol/kg, BIW), (Δ) EC2413 (500 nmol/kg, BIW)+500-fold excess EC0923, (□) EC1669 (500 nmol/kg, BIW) and (♦) EC2319 (500 nmol/kg, BIW).

FIG. 6A shows plasma concentration-time profiles for EC1669 and its metabolites (aminopterin gamma-hydrazide and aminopterin) when dosed subcutaneously in rats; (●) EC1669, (□) aminopterin gamma-hydrazide and (▴) aminopterin.

FIG. 6B shows plasma concentration-time profiles for EC2319 and its metabolite aminopterin when dosed subcutaneously in rats; (●) EC1669 and (▴) aminopterin.

FIG. 7 shows plasma concentration-time profiles for EC1669 and its metabolites (aminopterin gamma-hydrazide and aminopterin) when dosed subcutaneously in dogs; (●) EC1669, (□) aminopterin gamma-hydrazide and (▴) aminopterin.

FIG. 8A shows plasma concentration-time profiles for EC2319 and its metabolites (aminopterin and EC2496) when dosed intravenously in dogs; and subcutaneously in dogs; (●) EC2319, (▴) aminopterin and (□) EC2496.

FIG. 8B shows plasma concentration-time profiles for EC2319 and its metabolites (aminopterin and EC2496) when dosed intravenously in dogs; and subcutaneously in dogs; (●) EC2319, (▴) aminopterin and (□) EC2496.

FIG. 9A shows the release of aminopterin from EC1669 after incubation in rat, dog, and human liver cytosol at different pHs.

FIG. 9B shows the release of aminopterin from EC2319 after incubation in rat, dog, and human liver cytosol at different pHs.

FIG. 10 shows the release of aminopterin from EC1669 and EC2319 by gamma-glutamyl hydrolase.

FIG. 11A shows the release of aminopterin from EC1669 and EC2319 after incubation in rat TG macrophage cell lysates.

FIG. 11B shows the release of aminopterin from EC1669 and EC2319 after incubation in RAW264.7, THP-1 FRβ, and AIA rat macrophage cell lysates.

FIG. 12 shows plasma protein binding of EC1669 and EC2319. EC2319 exhibited higher plasma protein binding than did EC1669 in all species tested.

FIG. 13A shows stability of EC1669 and EC2319 in rat and human whole blood at 37° C.; (●) EC1669 Human, (□) EC2319 Human, (▴) EC1669 Rat and (♦) EC2319 Rat.

FIG. 13B shows aminopterin released after incubating EC1669 and EC2319 in rat and human whole blood at 37° C.; (●) EC1669 Human, (□) EC2319 Human, (▴) EC1669 Rat and (♦) EC2319 Rat.

### DEFINITIONS

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C≡C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e. C≡C). It will be understood that in certain embodiments alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkenyl may be unsubstituted, or substituted as described for alkyl or as described in the various embodiments provided herein. Illustrative alkenyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₆, C₃-C₆ and C₄-C₆.

Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like.

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like.

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like.

As used herein, “hydroxy” or ““hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “aryloxy” refers to an —O-aryl or an —O-heteroaryl group. Representative examples include, but are not limited to, phenoxy, pyridinyloxy, furanyloxy, thienyloxy, pyrimidinyloxy, pyrazinyloxy, and the like, and the like.

As used herein, “mercapto” refers to an —SH group.

As used herein, “alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

As used herein, “arylthio” refers to an —S-aryl or an —S-heteroaryl group. Representative examples include, but are not limited to, phenylthio, pyridinylthio, furanylthio, thienylthio, pyrimidinylthio, and the like.

As used herein, “halo” or “halogen” refers to fluorine, chlorine, bromine or iodine.

As used herein, “trihalomethyl” refers to a methyl group having three halo substituents, such as a trifluoromethyl group.

As used herein, “cyano” refers to a —CN group.

As used herein, “sulfinyl” refers to a —S(O)R″ group, where R″ is any R group as described in the various embodiments provided herein, or R″ may be a hydroxyl group.

As used herein, “sulfonyl” refers to a —S(O)₂R″ group, where R″ is any R group as described in the various embodiments provided herein, or R″ may be a hydroxyl group.

As used herein, “S-sulfonamido” refers to a —S(O)₂NR″R″ group, where R″ is any R group as described in the various embodiments provided herein.

As used herein, “N-sulfonamido” refers to a —NR″S(O)₂R″ group, where R″ is any R group as described in the various embodiments provided herein.

As used herein, “O-carbamyl” refers to a —OC(O)NR″R″ group, where R″ is any R group as described in the various embodiments provided herein.

As used herein, “N-carbamyl” refers to an R″OC(O)NR″— group, where R″ is any R group as described in the various embodiments provided herein.

As used herein, “O-thiocarbamyl” refers to a —OC(S)NR″R″ group, where R″ is any R group as described in the various embodiments provided herein.

As used herein, “N-thiocarbamyl” refers to a R″OC(S)NR″— group, where R″ is any R group as described in the various embodiments provided herein.

As used herein, “amino” refers to an —NR″R″ group, where R″ is any R group as described in the various embodiments provided herein.

As used herein, “C-amido” refers to a —C(O)NR″R″ group, where R″ is any R group as described in the various embodiments provided herein.

As used herein, “N-amido” refers to a R″C(O)NR″— group, where R″ is any R group as described in the various embodiments provided herein.

As used herein, “nitro” refers to a —NO₂ group.

As used herein, “bond” refers to a covalent bond.

As used herein, “optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocycle group is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the term “pharmaceutically acceptable salt” refers to those salts with counter ions which may be used in pharmaceuticals. Such salts include:
(1) acid addition salts, which can be obtained by reaction of the free base of the parent conjugate with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent conjugate either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.
   Pharmaceutically acceptable salts are well known to those skilled in the art, and any such pharmaceutically acceptable salt may be contemplated in connection with the embodiments described herein.

As used herein, “amino acid” (a.k.a. “AA”) means any molecule that includes an alpha-carbon atom covalently bonded to an amino group and an acid group. The acid group may include a carboxyl group. “Amino acid” may include molecules having one of the formulas:

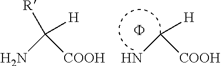

wherein R′ is a side group and P includes at least 3 carbon atoms. “Amino acid” includes stereoisomers such as the D-amino acid and L-amino acid forms. Illustrative amino acid groups include, but are not limited to, the twenty human amino acids and their derivatives, such as lysine (Lys), asparagine (Asn), threonine (Thr), serine (Ser), isoleucine (Ile), methionine (Met), proline (Pro), histidine (His), glutamine (Gln), arginine (Arg), glycine (Gly), aspartic acid (Asp), glutamic acid (Glu), alanine (Ala), valine (Val), phenylalanine (Phe), leucine (Leu), tyrosine (Tyr), cysteine (Cys), tryptophan (Trp), phosphoserine (PSER), sulfo-cysteine, arginosuccinic acid (ASA), hydroxyproline, phosphoethanolamine (PEA), sarcosine (SARC), taurine (TAU), carnosine (CARN), citrulline (CIT), anserine (ANS), 1,3-methyl-histidine (ME-HIS), alpha-amino-adipic acid (AAA), beta-alanine (BALA), ethanolamine (ETN), gamma-amino-butyric acid (GABA), beta-amino-isobutyric acid (BAIA), alpha-amino-butyric acid (BABA), L-allo-cystathionine (cystathionine-A; CYSTA-A), L-cystathionine (cystathionine-B; CYSTA-B), cystine, allo-isoleucine (ALLO-ILE), DL-hydroxylysine (hydroxylysine (I)), DL-allo-hydroxylysine (hydroxylysine (2)), ornithine (ORN), homocystine (HCY), and derivatives thereof. It will be appreciated that each of these examples are also contemplated in connection with the present disclosure in the D-configuration as noted above. Specifically, for example, D-lysine (D-Lys), D-asparagine (D-Asn), D-threonine (D-Thr), D-serine (D-Ser), D-isoleucine (D-Ile), D-methionine (D-Met), D-proline (D-Pro), D-histidine (D-His), D-glutamine (D-Gln), D-arginine (D-Arg), D-glycine (D-Gly), D-aspartic acid (D-Asp), D-glutamic acid (D-Glu), D-alanine (D-Ala), D-valine (D-Val), D-phenylalanine (D-Phe), D-leucine (D-Leu), D-tyrosine (D-Tyr), D-cysteine (D-Cys), D-tryptophan (D-Trp), D-citrulline (D-CIT), D-carnosine (D-CARN), and the like. In connection with the embodiments described herein, amino acids can be covalently attached to other portions of the conjugates described herein through their alpha-amino and carboxy functional groups (i.e. in a peptide bond configuration), or through their side chain functional groups (such as the side chain carboxy group in glutamic acid) and either their alpha-amino or carboxy functional groups. It will be understood that amino acids, when used in connection with the conjugates described herein, may exist as zwitterions in a conjugate in which they are incorporated.

As used herein, “sugar” refers to carbohydrates, such as monosaccharides, disaccharides, or oligosaccharides. In connection with the present disclosure, monosaccharides are preferred. Non-limiting examples of sugars include erythrose, threose, ribose, arabinose, xylose, lyxose, allose, altrose, glucose, mannose, galactose, ribulose, fructose, sorbose, tagatose, and the like. It will be understood that as used in connection with the present disclosure, sugar includes cyclic isomers of amino sugars, deoxy sugars, acidic sugars, and combinations thereof. Non-limiting examples of such sugars include, galactosamine, glucosamine, deoxyribose, fucose, rhamnose, glucuronic acid, ascorbic acid, and the like. In some embodiments, sugars for use in connection with the present disclosure include

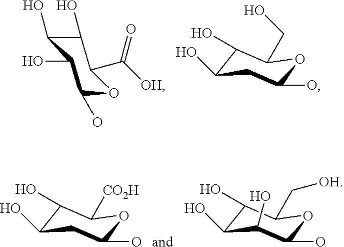

As used herein, “prodrug” refers to a compound that can be administered to a subject in a pharmacologically inactive form which then can be converted to a pharmacologically active form through a normal metabolic process, such as hydrolysis of an oxazolidine. It will be understood that the metabolic processes through which a prodrug can be converted to an active drug include, but are not limited to, one or more spontaneous chemical reaction(s), enzyme-catalyzed chemical reaction(s), and/or other metabolic chemical reaction(s), or a combination thereof. It will be appreciated that a variety of metabolic processes are known in the art, and the metabolic processes through which the prodrugs described herein are converted to active drugs are non-limiting. A prodrug can be a precursor chemical compound of a drug that has a therapeutic effect on a subject.

As used herein, the term “therapeutically effective amount” refers to an amount of a drug or pharmaceutical agent that elicits the biological or medicinal response in a subject (i.e. a tissue system, animal or human) that is being sought by a researcher, veterinarian, medical doctor or other clinician, which includes, but is not limited to, alleviation of the symptoms of the disease or disorder being treated. In one aspect, the therapeutically effective amount is that amount of an active which compound may treat or alleviate the disease or symptoms of the disease at a reasonable benefit/risk ratio applicable to any medical treatment. In another aspect, the therapeutically effective amount is that amount of an inactive prodrug which when converted through normal metabolic processes produces an amount of active drug capable of eliciting the biological or medicinal response in a subject that is being sought.

It is also appreciated that the dose, whether referring to monotherapy or combination therapy, is advantageously selected with reference to any toxicity, or other undesirable side effect, that might occur during administration of one or more of the conjugates described herein. Further, it is appreciated that the co-therapies described herein may allow for the administration of lower doses of conjugates that show such toxicity, or other undesirable side effect, where those lower doses are below thresholds of toxicity or lower in the therapeutic window than would otherwise be administered in the absence of a co-therapy.

As used herein, “administering” includes all means of introducing the conjugates and compositions described herein to the patient, including, but are not limited to, oral (po), intravenous (iv), intramuscular (im), subcutaneous (sc), transdermal, inhalation, buccal, ocular, sublingual, vaginal, rectal, and the like. The conjugates and compositions described herein may be administered in unit dosage forms and/or formulations containing conventional nontoxic pharmaceutically-acceptable carriers, adjuvants, and/or vehicles.

As used herein “pharmaceutical composition” or “composition” refers to a mixture of one or more of the conjugates described herein, or pharmaceutically acceptable salts, solvates, hydrates thereof, with other chemical components, such as pharmaceutically acceptable excipients. The purpose of a pharmaceutical composition is to facilitate administration of a conjugate to a subject. Pharmaceutical compositions suitable for the delivery of conjugates described and methods for their preparation will be readily apparent to those skilled in the art. Such compositions and methods for their preparation may be found, for example, in ‘Remington's Pharmaceutical Sciences’, 19th Edition (Mack Publishing Company, 1995).

A “pharmaceutically acceptable excipient” refers to an inert substance added to a pharmaceutical composition to further facilitate administration of a conjugate such as a diluent or a carrier.

### DETAILED DESCRIPTION

In each of the foregoing and each of the following embodiments, it is to be understood that the formulae include and represent not only all pharmaceutically acceptable salts of the conjugates, but also include any and all hydrates and/or solvates of the conjugate formulae. It is appreciated that certain functional groups, such as the hydroxy, amino, and like groups form complexes and/or coordination conjugates with water and/or various solvents, in the various physical forms of the conjugates. Accordingly, the above formulae are to be understood to include and represent those various hydrates and/or solvates. It is also to be understood that the non-hydrates and/or non-solvates of the conjugate formulae are described by such formula, as well as the hydrates and/or solvates of the conjugate formulae.

The conjugates described herein can be expressed by the generalized descriptors B, L and D¹, for example B-L-D¹, where B is a cell surface receptor binding ligand (a.k.a. a “binding ligand”), L is a linker that may include one or more releasable portions (i.e. a releasable linker) and L may be described by, for example, one or more of the groups AA, L¹ or L² as defined herein, and D¹ represents a drug covalently attached to the conjugates described herein.

The conjugates described herein can be described according to various embodiments including but not limited to B-L¹-AA-L¹-AA-L¹-L²-D¹, B-AA-L¹-AA-AA-L²-D¹, or B-AA-AA-AA-AA-L²-D¹, wherein B, AA, L¹, L² and D¹ are defined by the various embodiments described herein, or a pharmaceutically acceptable salt thereof.

As used herein, the term cell surface receptor binding ligand (aka a “binding ligand”), generally refers to compounds that bind to and/or target receptors that are found on cell surfaces, and in particular those that are found on, over-expressed by, and/or preferentially expressed on the surface of pathogenic cells, such as inflammation. Illustrative ligands include, but are not limited to, vitamins and vitamin receptor binding compounds.

Illustrative vitamin moieties include carnitine, inositol, lipoic acid, pyridoxal, ascorbic acid, niacin, pantothenic acid, folic acid, riboflavin, thiamine, biotin, vitamin B₁₂, and the lipid soluble vitamins A, D, E and K. These vitamins, and their receptor-binding analogs and derivatives, constitute the targeting entity covalently attached to the linker. Illustrative biotin analogs that bind to biotin receptors include, but are not limited to, biocytin, biotin sulfoxide, oxybiotin, and the like).

Illustrative folic acid analogs that bind to folate receptors include, but are not limited to folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. The foregoing folic acid analogs and/or derivatives are conventionally termed “folates,” reflecting their ability to bind to folate-receptors, and such ligands when conjugated with exogenous molecules are effective to enhance transmembrane transport, such as via folate-mediated endocytosis as described herein.

In some embodiments, B is of the formula I

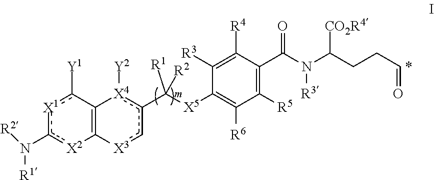

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;

X¹ is —NR¹¹—, ═N—, —N═, —C(R¹¹)═ or ═C(R¹¹)—;

X² is —NR^{11′}— or ═N—;

X³ is —NR^{11″}—, —N═ or —C(R^{11′})═;

X⁴ is —N═ or —C═;

X⁵ is NR¹² or CR¹²R^{12′};

Y¹ is H, D, —OR¹³ or —SR¹³ when X¹ is —N═ or —C(R¹¹)═, or Y¹ is ═O when X¹ is —NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;

R^{1′}, R^{2′}, R^{3′}, R^{4′}, R¹¹, R^{11′}, R^{11″}, R¹², R^{12′}, R¹³, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

As used herein, L¹ can be any group covalently attaching portions of the linker to the binding ligand, portions of the linker to other portions of the linker, or portions of the linker to D¹. It will be understood that the structure of L¹ is not particularly limited in any way. It will be further understood that L¹ can comprise numerous functionalities well known in the art to covalently attach portions of the linker to the binding ligand, portions of the linker to other portions of the linker, or portions of the linker to D¹, including but not limited to, alkyl groups, ether groups, amide groups, carboxy groups, sulfonate groups, alkenyl groups, alkynyl groups, cycloalkyl groups, aryl groups, heterocycloalkyl, heteroaryl groups, and the like. In some embodiments, L¹ is a linker of the formula II

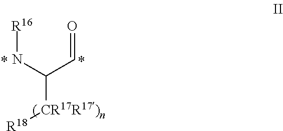

wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R²⁹;

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

It will be appreciated that when L¹ is described according to the formula II, that both the R- and S-configurations are contemplated. In some embodiments, L¹ is of the formula IIa or IIb

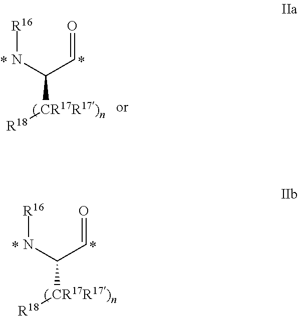

where each of R¹⁶, R¹⁷, R^{17′}, R¹⁸, n and * are as defined for the formula II.

In some embodiments, each L¹ is selected from the group consisting of

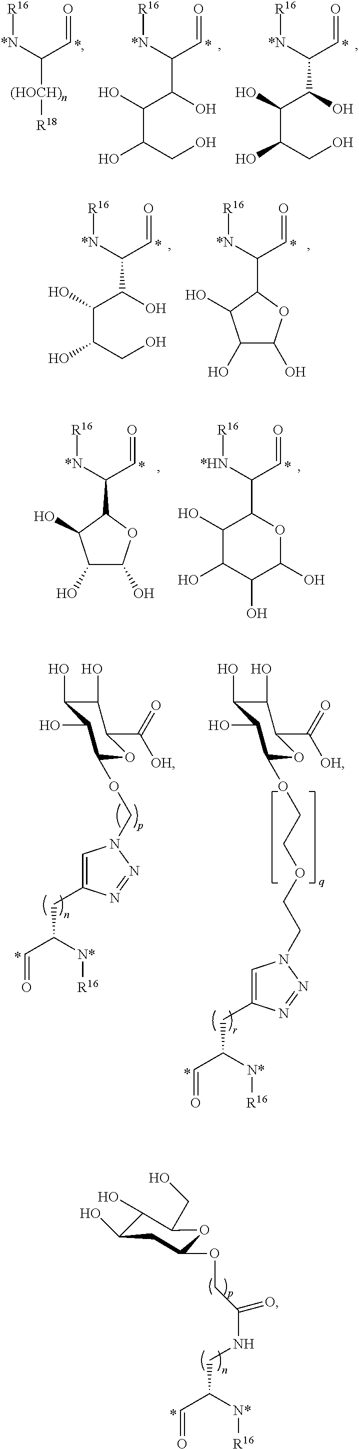

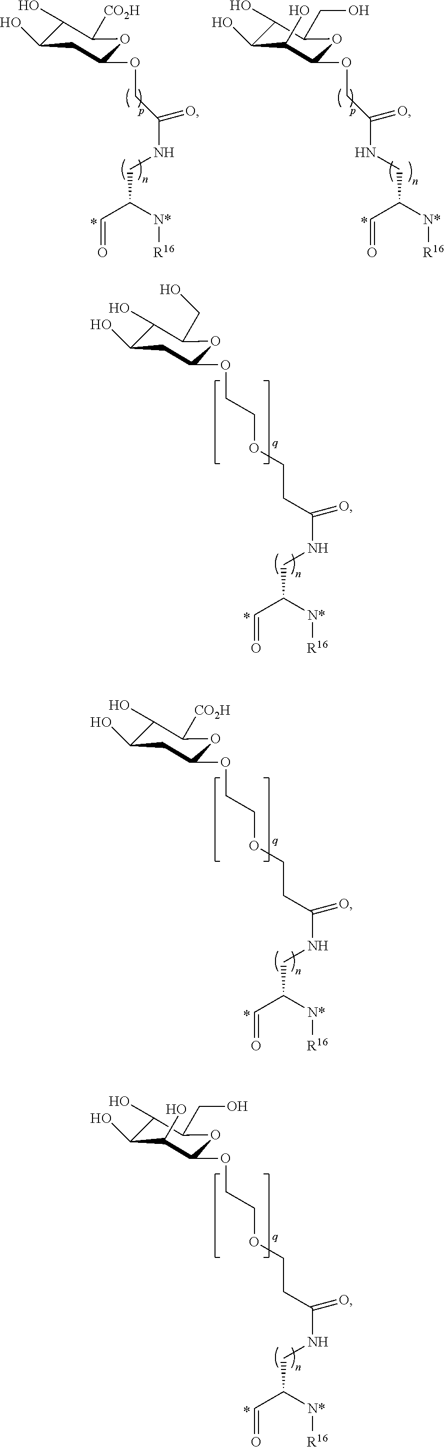

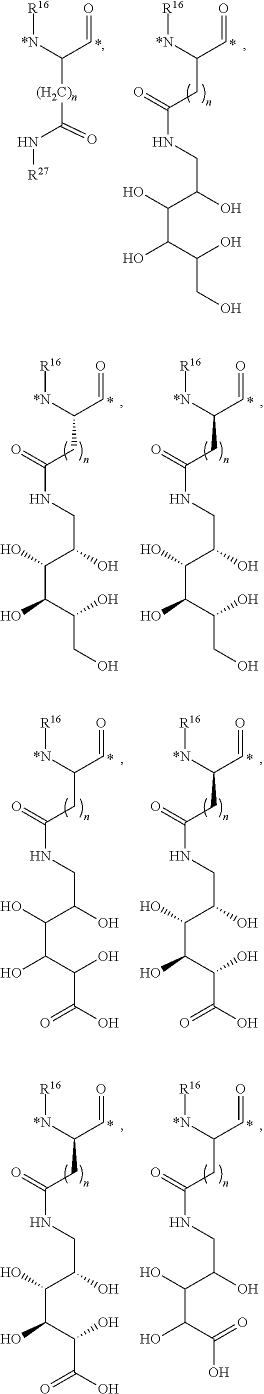

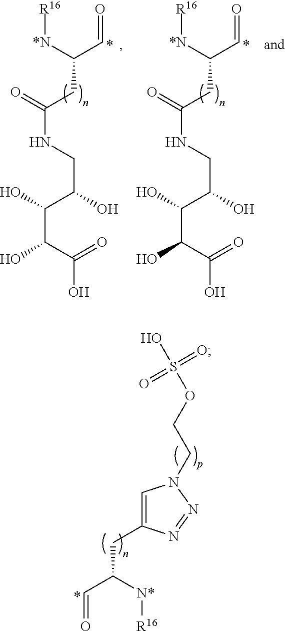

and combinations thereof,
wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, each L¹ is selected from the group consisting of

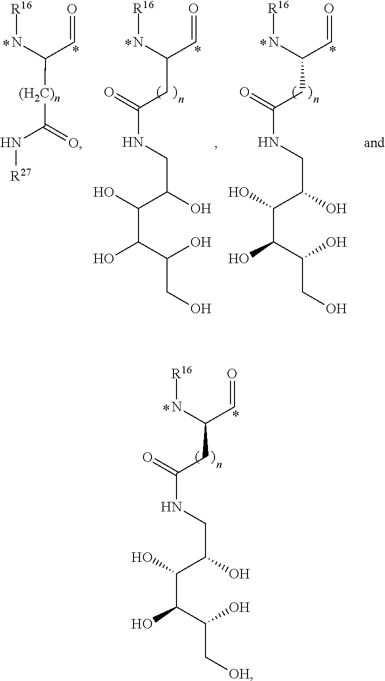

wherein R¹⁶ is defined as described herein, and * is a covalent bond.

In some embodiments, R¹⁶ is H. In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is a H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹, each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;

R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);

R²⁸ is H or sugar;

n is 1, 2, 3, 4 or 5;

p is 1, 2, 3, 4 or 5;

q is 1, 2, 3, 4 or 5; and

* is a covalent bond.

In some embodiments, each L¹ is selected from the group consisting of

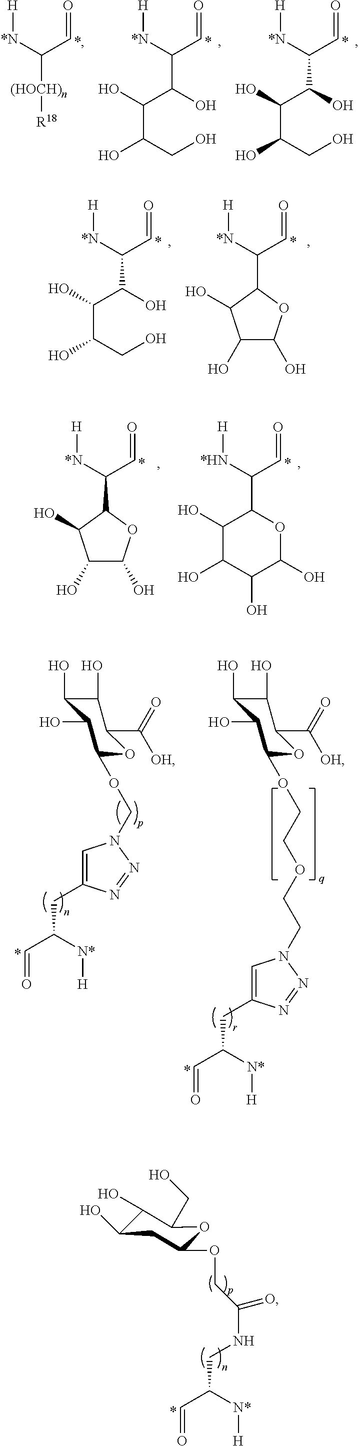

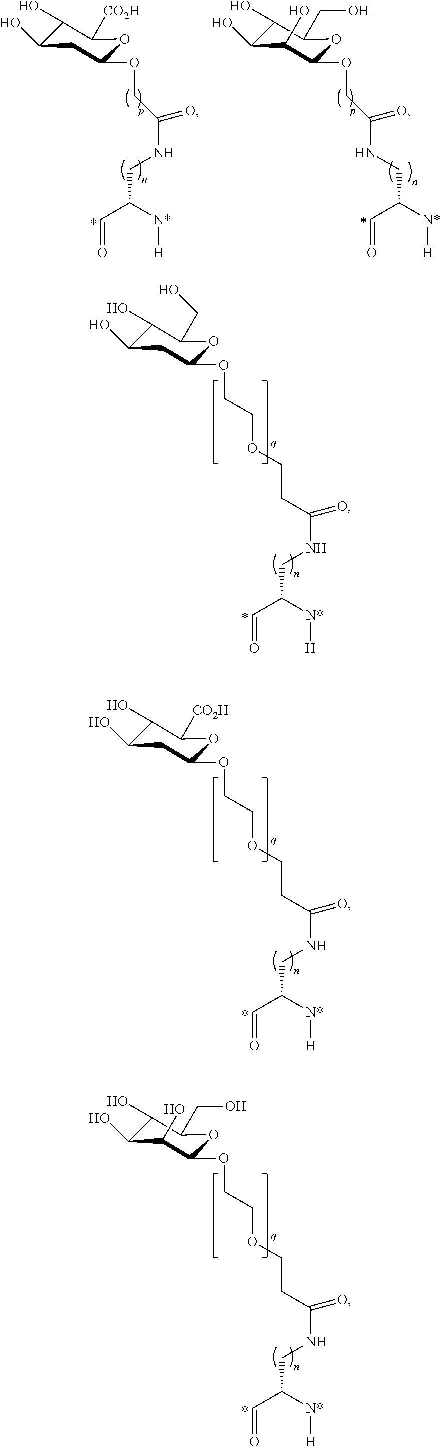

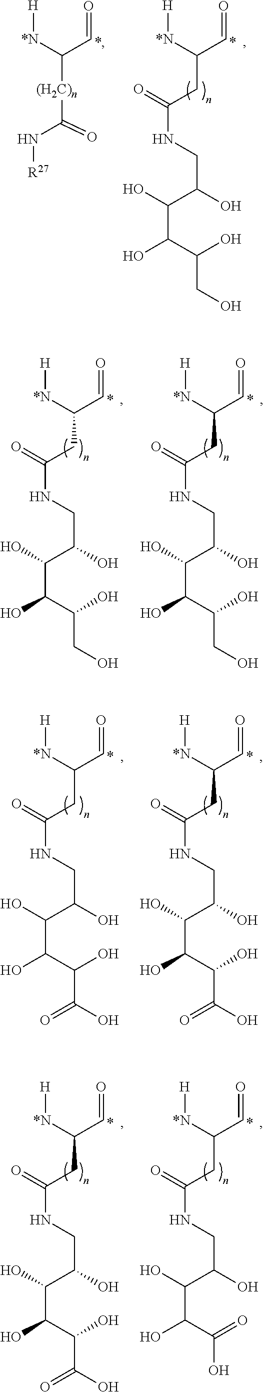

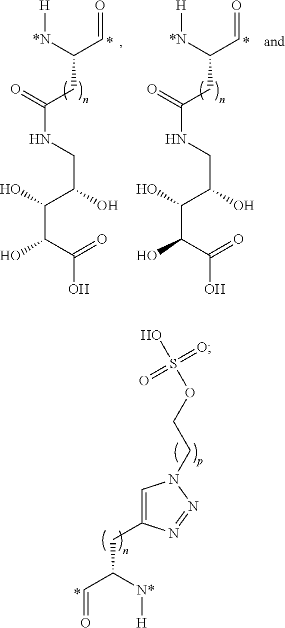

and combinations thereof,

wherein

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is a H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);

R²⁸ is a H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹, each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;

R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);

R²⁸ is H or sugar;

n is 1, 2, 3, 4 or 5;

p is 1, 2, 3, 4 or 5;

q is 1, 2, 3, 4 or 5; and

* is a covalent bond.

AA is an amino acid as described herein. In certain embodiments, AA is a naturally occurring amino acid. In certain embodiments, AA is in the L-form. In certain embodiments, AA is in the D-form. In other embodiments, AA is an unnatural amino acid. It will be appreciated that in certain embodiments, the conjugates described herein will comprise more than one amino acid as portions of the linker, and the amino acids can be the same or different, and can be selected from a group of amino acids. It will be appreciated that in certain embodiments, the conjugates described herein will comprise more than one amino acid as portions of the linker, and the amino acids can be the same or different, and can be selected from a group of amino acids in D- or L-form. In some embodiments, at least one AA is in the L-configuration. In some embodiments, at least two AA are in the L-configuration. In some embodiments, at least one AA is in the D-configuration. In some embodiments, at least two AA are in the D-configuration. In some embodiments, each AA is independently selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

In some embodiments, each AA is independently selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline. In some embodiments, each AA is independently selected from the group consisting of L-arginine, D-arginine, L-aspartic acid, D-aspartic acid, L-glutamic acid and D-glutamic acid.

L² is a releasable linker. As used herein, the term “releasable linker” refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It is understood that a cleavable bond can connect two adjacent atoms within the releasable linker and/or connect other linkers, B or D¹, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, such as another linker, a drug or binding ligand, the releasable linker becomes separated from the other moiety following breaking of the bond.

The lability of the cleavable bond can be adjusted by, for example, substituents at or near the cleavable bond, such as including alpha-branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like.

In some embodiments, releasable linkers described herein include one or more cleavable functional groups, such as a disulfide, a carbonate, a carbamate, an amide, an ester, and the like. Illustrative releasable linkers described herein include linkers that include hemiacetals and sulfur variations thereof, acetals and sulfur variations thereof, hemiaminals, aminals, and the like, and can be formed from methylene fragments substituted with at least one heteroatom, 1-alkoxyalkylene, 1-alkoxycycloalkylene, 1-alkoxyalkylenecarbonyl, 1-alkoxycycloalkylene-carbonyl, and the like. Illustrative releasable linkers described herein include linkers that include carbonylarylcarbonyl, carbonyl(carboxyaryl)carbonyl, carbonyl(biscarboxyaryl)carbonyl, haloalkylenecarbonyl, and the like. Illustrative releasable linkers described herein include linkers that include alkylene(dialkylsilyl), alkylene(alkylarylsilyl), alkylene(diarylsilyl), (dialkylsilyl)aryl, (alkylarylsilyl)aryl, (diarylsilyl)aryl, and the like. Illustrative releasable linkers described herein include oxycarbonyloxy, oxycarbonyloxyalkyl, sulfonyloxy, oxysulfonylalkyl, and the like. Illustrative releasable linkers described herein include linkers that include iminoalkylidenyl, carbonylalkylideniminyl, iminocycloalkylidenyl, carbonylcycloalkyliden-iminyl, and the like.

Illustrative releasable linkers described herein include linkers that include alkylenethio, alkylenearylthio, and carbonylalkylthio, and the like.

In some embodiments, the conjugates described herein comprise more than one releasable linker. It will be appreciated that when the conjugates described herein comprise more than one releasable linker, the releasable linkers may be the same. It will be further appreciated that when the conjugates described herein comprise more than one releasable linker, the releasable linkers may be different. In some embodiments, the conjugates described herein comprise more than one releasable linker, wherein the more than one releasable linker comprises in each instance a disulfide bond. In some embodiments, the conjugates described herein comprise two releasable linkers both of which include a disulfide bond.

In some embodiments, L² is of the formula

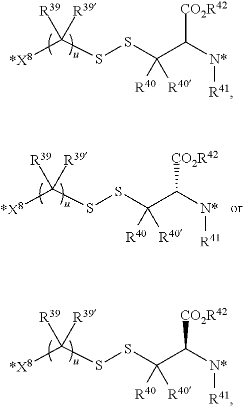

wherein

X⁸ is —NR⁵⁰— or —O—;

each R³⁹, R^{39′}, R⁴⁰ and R^{40′} is independently selected from the group consisting of H, D, C₁-C₆ alkyl, C₂-C₆ alkenyl, C₂-C₆ alkynyl, C₃-C₆ cycloalkyl, —OR⁴⁸, —OC(O)R⁴⁸, —OC(O)NR⁴⁸R^{48′}, —OS(O)R⁴⁸, —OS(O)₂R⁴⁸, —SR⁴⁸, —S(O)R⁴⁸, —S(O)₂R⁴⁸, —S(O)NR⁴⁸R^{48′}, —S(O)₂NR⁴⁸R^{48′}, —OS(O)NR⁴⁸R^{48′}, —OS(O)₂NR⁴⁸R^{48′}, —NR⁴⁸R^{48′}, —NR⁴⁸C(O)R⁴⁹, —NR⁴⁸C(O)OR⁴⁹, —NR⁴⁸C(O)NR⁴⁹R^{49′}, —NR⁴⁸S(O)R⁴⁹, —NR⁴⁸S(O)₂R⁴⁹, —NR⁴⁸S(O)NR⁴⁹R^{49′}, —NR⁴⁸S(O)₂NR⁴⁹R^{49′}, —C(O)R⁴⁸, —C(O)OR⁴⁸ or —C(O)NR⁴⁸R^{48′}, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR⁴⁴, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};

each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};

each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′};

each R⁴³, R^{43′}, R⁴⁴, R^{44′}, R⁴⁵, R^{45′}, R⁴⁶, R^{46′}, R⁴⁷, R^{47′}, R⁴⁸, R^{48′}, R⁴⁹, R^{49′} and R⁵⁰ is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl; u is 1, 2, 3 or 4; and each * is a covalent bond.

In some embodiments, L² is of the formula

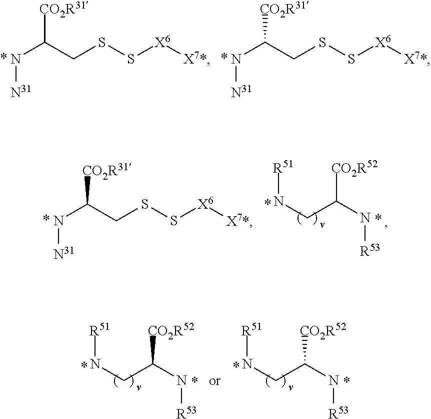

wherein

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};

each X⁷ is —NR^{31a}— or —O—, and when X⁶ is C₁-C₆ alkyl and X⁷ is —O—, then at least one hydrogen atom in C₁-C₆ alkyl is substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};

each R³¹ and R^{31a} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a}, —OS(O)₂NR^{32a}R^{32a′}, —NR^{32a}R^{32a′}, —C(O)R^{32a}, —C(O)OR^{32a} or —C(O)NR^{32a}R^{32a′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

each R⁵¹ and R⁵³ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁴, —OC(O)R⁵⁴, —OC(O)NR⁵⁴R^{54′}, —OS(O)R⁵⁴, —OS(O)₂R⁵⁴, —SR⁵⁴, —S(O)R⁵⁴, —S(O)₂R⁵⁴, —S(O)NR⁵⁴R^{54′}, —S(O)₂NR⁵⁴R^{54′}, —OS(O)NR⁵⁴R^{54′}, —OS(O)₂NR⁵⁴R^{54′}, —NR⁵⁴R^{54′}, —NR⁵⁴C(O)R⁵⁵, —NR⁵⁴C(O)OR⁵⁵, —NR⁵⁴C(O)NR⁵⁵R^{55′}, —NR⁵⁴S(O)R⁵⁵, —NR⁵⁴S(O)₂R⁵⁵, —NR⁵⁴S(O)NR⁵⁵R^{55′}, —NR⁵⁴S(O)₂NR⁵⁵R^{55′}, —C(O)R⁵⁴, —C(O)OR⁵⁴ or —C(O)NR⁵⁴R^{54′};

each R⁵² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁵⁶, —OC(O)R⁵⁶, —OC(O)NR⁵⁶R^{56′}, —OS(O)R⁵⁶, —OS(O)₂R⁵⁶, —SR⁵⁶, —S(O)R⁵⁶, —S(O)₂R⁵⁶, —S(O)NR⁵⁶R^{56′}, —S(O)₂NR⁵⁶R^{56′}, —OS(O)NR⁵⁶R^{56′}, —OS(O)₂NR⁵⁶R^{56′}, —NR⁵⁶R^{56′}, —C(O)R⁵⁶, —C(O)OR⁵⁶ or —C(O)NR⁵⁶R^{56′};

each R⁵⁴, R^{54′}, R⁵⁵, R^{55′}, R⁵⁶ and R^{56′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

v is 1, 2, 3, 4, 5 or 6; and

each * is a covalent bond.

In some embodiments, the linker is of the formula -L¹-AA-L¹-AA-L¹-L²-having the formula

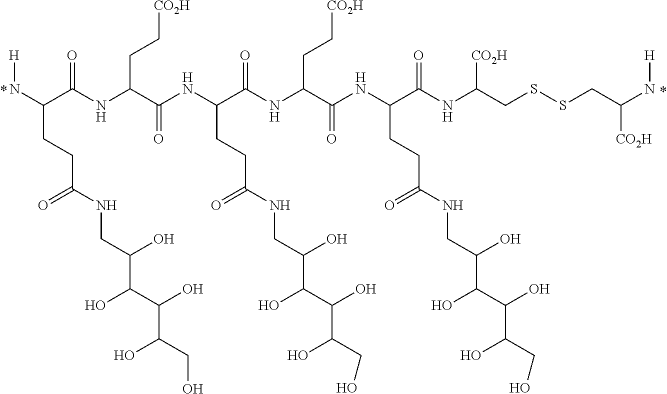 [SMILES: NC(CSSCC(NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(N)CCC(=O)NCC(O)C(O)C(O)C(O)CO)C(=O)O)C(=O)O]

wherein each * is a covalent bond to B or D¹.

In some embodiments, the linker is of the formula -L¹-AA-L¹-AA-L¹-L²-having the formula

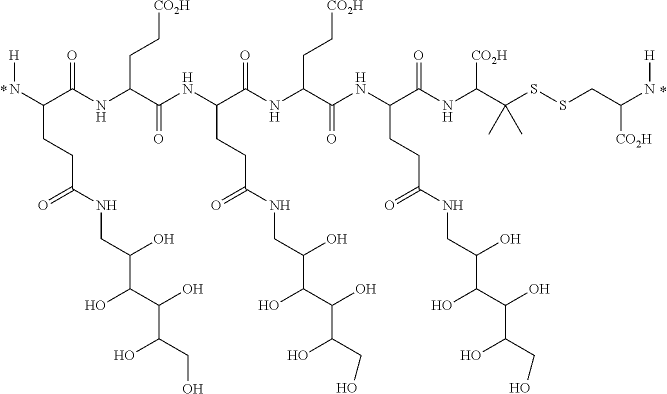 [SMILES: CC(C)(SSCC(N)C(=O)O)C(NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(N)CCC(=O)NCC(O)C(O)C(O)C(O)CO)C(=O)O]

wherein each * is a covalent bond to B or D¹.

In some embodiments, the linker is of the formula -L¹-AA-L¹-AA-L¹-L²-having the formula

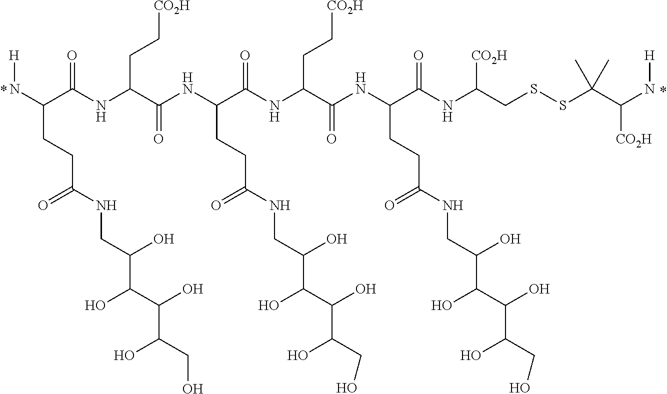 [SMILES: CC(C)(SSCC(NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(N)CCC(=O)NCC(O)C(O)C(O)C(O)CO)C(=O)O)C(N)C(=O)O]

wherein each * is a covalent bond to B or D¹.

In some embodiments, the linker is of the formula -L¹-AA-L¹-AA-L¹-L²-having the formula

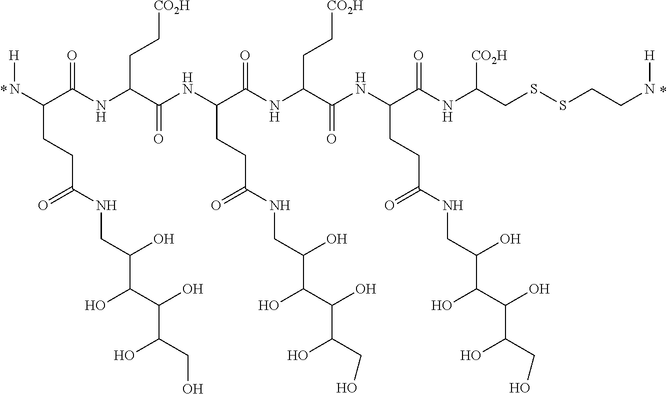 [SMILES: NCCSSCC(NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(N)CCC(=O)NCC(O)C(O)C(O)C(O)CO)C(=O)O]

wherein each * is a covalent bond to B or D¹.

In some embodiments, the linker is of the formula -L¹-AA-L¹-AA-L¹-L²-having the formula

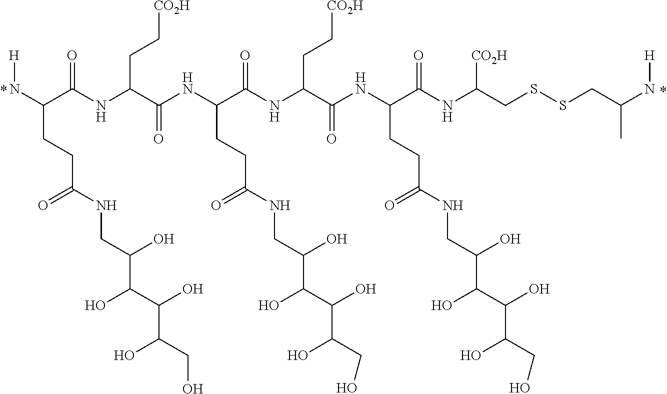 [SMILES: CC(N)CSSCC(NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(N)CCC(=O)NCC(O)C(O)C(O)C(O)CO)C(=O)O]

wherein each * is a covalent bond to B or D¹.

In some embodiments, the linker is of the formula -L¹-AA-L¹-AA-L¹-L²-having the formula

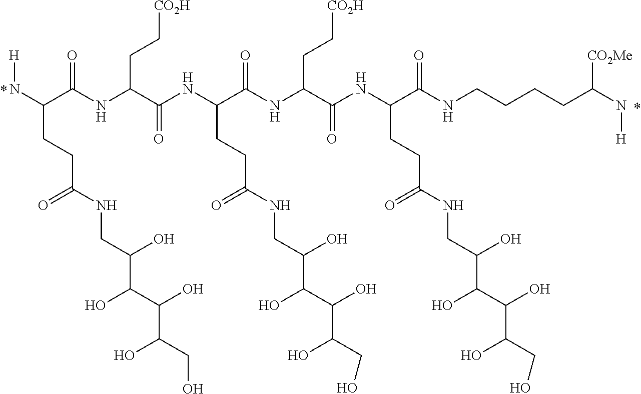 [SMILES: COC(=O)C(N)CCCCNC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(N)CCC(=O)NCC(O)C(O)C(O)C(O)CO]

wherein each * is a covalent bond to B or D¹.

In some embodiments, the linker is of the formula -AA-AA-AA-AA-L²-having the formula

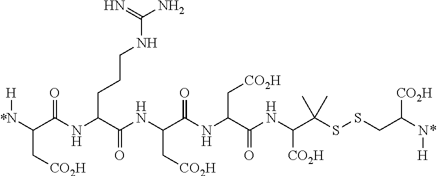 [SMILES: CC(C)(SSCC(N)C(=O)O)C(NC(=O)C(CC(=O)O)NC(=O)C(CC(=O)O)NC(=O)C(CCCNC(=N)N)NC(=O)C(N)CC(=O)O)C(=O)O]

wherein each * is a covalent bond to B or D¹.

In some embodiments, the linker is of the formula -AA-AA-AA-AA-L²-having the formula

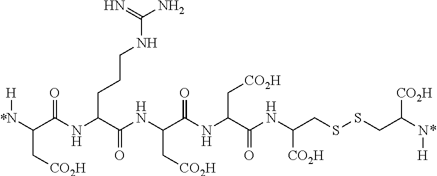 [SMILES: N=C(N)NCCCC(NC(=O)C(N)CC(=O)O)C(=O)NC(CC(=O)O)C(=O)NC(CC(=O)O)C(=O)NC(CSSCC(N)C(=O)O)C(=O)O]

wherein each * is a covalent bond to B or D¹.

In some embodiments, the linker is of the formula -AA-AA-AA-AA-L²-having the formula

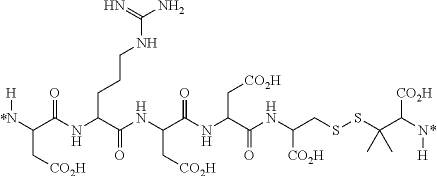 [SMILES: CC(C)(SSCC(NC(=O)C(CC(=O)O)NC(=O)C(CC(=O)O)NC(=O)C(CCCNC(=N)N)NC(=O)C(N)CC(=O)O)C(=O)O)C(N)C(=O)O]

wherein each * is a covalent bond to B or D¹.

In some embodiments, D¹ is of the formula III

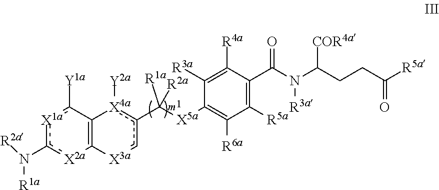

wherein

R^{1a} and R^{2a} in each instance are independently selected from the group consisting of H, D, halogen, C₁-C₆ alkyl, C₂-C₆ alkenyl, C₂-C₆ alkynyl, —OR^{7a}, —SR^{7a} and —NR^{7a}R^{7a′}, wherein each hydrogen atom in C₁-C₆ alkyl, C₂-C₆ alkenyl and C₂-C₆ alkynyl is independently optionally substituted by halogen, —OR^{8a}, —SR^{8a}, —NR^{8a}R^{8a′}, —C(O)R^{8a}, —C(O)OR^{8a} or —C(O)NR^{8a}R^{8a′};

R^{3a}, R^{4a}, R^{5a} and R^{6a} are each independently selected from the group consisting of H, D, halogen, C₁-C₆ alkyl, C₂-C₆ alkenyl, C₂-C₆ alkynyl, —CN, —NO₂, —NCO, —OR^{9a}, —SR^{9a}, —NR^{9a}R^{9a′}, —C(O)R^{9a}, —C(O)OR^{9a} and —C(O)NR^{9a}R^{9a′}, wherein each hydrogen atom in C₁-C₆ alkyl, C₂-C₆ alkenyl and C₂-C₆ alkynyl is independently optionally substituted by halogen, —OR^{10a}, —SR^{10a}, —NR^{10a}R^{10a′}, —C(O)R^{10a}, —C(O)OR^{10a} or —C(O)NR^{10a}R^{10a′};

each R^{7a}, R^{7a′}, R^{8a}, R^{8a′}, R^{9a}, R^{9a′}, R^{10a} and R^{10a′} is independently H, D, C₁-C₆ alkyl, C₂-C₆ alkenyl or C₂-C₆ alkynyl;

X^{1a} is —NR^{11a}—, ═N—, —N═, —C(R^{11a})═ or ═C(R^{11a})—;

X^{2a} is —NR^{11a′}— or ═N—;

X^{3a} is —NR^{11a″}—, —N═ or —C(R^{11a′})═;

X^{4a} is —N═ or —C═;

X^{5a} is —NR^{12a}— or —CR^{12a}R^{12a′}—;

Y^{1a} is —NR^{13a}R^{13a′} when X^{1a} is —N═ or —C(R^{11a})═, or Y^{1a} is ═NR^{13a} when X^{1a} is —NR^{11a}—, ═N— or ═C(R^{11a})—;

Y^{2a} is H, D, C₁-C₆ alkyl, C₂-C₆ alkenyl, —C(O)R^{14a}, —C(O)OR^{14a} or —C(O)NR^{14a}R^{14a′} when X^{4a} is —C═, or Y^{2a} is absent when X^{4a} is —N═;

R^{1a′}, R^{2a′}, R^{3a′}, R^{11a}, R^{11a′}, R^{11a″}, R^{12a}, R^{12a′}, R^{13a}, R^{13a′}, R^{14a} and R^{14a′} are each independently selected from the group consisting of H, D, C₁-C₆ alkyl, C₂-C₆ alkenyl, C₂-C₆ alkynyl, —C(O)R^{15a}, —C(O)OR^{15a} and —C(O)NR^{15a}R^{15a′};

R^{4a′} and R^{5a′} are each independently selected from the group consisting of C₁-C₆ alkyl, C₂-C₆ alkenyl, C₂-C₆ alkynyl, —OR^{16a}, —SR^{16a}, —NR^{16a}R^{16a′}, provided that one of R^{4a′} and R^{5a′} is a covalent bond to an AA, a L¹ or a L²;

R^{15a}, R^{15a′}, R^{16a} and R^{16a′} are each independently H or C₁-C₆ alkyl;

m¹ is 1, 2, 3 or 4; and

each * is a covalent bond.

The conjugates described herein can be used for both human clinical medicine and veterinary applications. Thus, the patient harboring the population of pathogenic cells and treated with the conjugates described herein can be human or, in the case of veterinary applications, can be a laboratory, agricultural, domestic, or wild animal. The conjugates described herein can be applied to patients including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The methods are applicable to populations of pathogenic cells that cause inflammation. For example, activated macrophages or activated monocytes capable of causing a disease state, such as inflammation, can be reduced in number, eliminated, or their activity inhibited because they uniquely express, preferentially express, or overexpress folate receptors, or receptors that bind analogs or derivatives of folate. For example, the pathogenic cells can be inflammatory cells that are pathogenic under some circumstances such as cells of the immune system that are responsible for graft versus host disease, but not pathogenic under other circumstances.

In some embodiment, folates, or analogs or derivatives thereof that can be used in the conjugates described herein include those that bind to folate receptors expressed specifically on activated macrophages or activated monocytes. The conjugates described herein can be used to kill, eliminate, reduce in number or suppress the activity of activated macrophages or activated monocytes that cause disease states in the patient. Without being bound by theory, it is believed that the conjugates described herein, when administered to a patient suffering from inflammation, work to concentrate and associate the conjugated drug with the population of inflammatory cells, thus providing a means to kill, eliminate or reduce in number, the inflammatory cells, or suppress their function. Elimination, reduction, or deactivation of the inflammatory cell population can stop or reduce the pathogenic characteristic of the disease state being treated. Exemplary inflammatory diseases include arthritis, including rheumatoid arthritis and osteoarthritis, glomerulonephritis, proliferative retinopathy, restenosis, ulcerative colitis, Crohn's disease, fibromyalgia, psoriasis and other inflammations of the skin, inflammations of the eye, including uveitis and autoimmune uveitis, osteomyelitis, Sögren's syndrome, multiple sclerosis, diabetes, atherosclerosis, pulmonary fibrosis, lupus erythematosus, sarcoidosis, systemic sclerosis, organ transplant rejection (GVHD) and chronic inflammations. Administration of a conjugate as described herein can be continued until symptoms of the disease state are reduced or eliminated.

As used herein the term uveitis generally refers to an intraocular inflammatory disease including iritis, cyclitis, panuveits, posterior uveitis and anterior uveitis. Iritis is inflammation of the iris. Cyclitis is inflammation of the ciliary body. Panuveitis refers to inflammation of the entire uveal (vascular) layer of the eye. Intermediate uveitis, also called peripheral uveitis, is centered in the area immediately behind the iris and lens in the region of the ciliary body and pars plana, and is also termed “cyclitis” and “pars planitis.”

Autoimmune uveitis may occur as a component of an autoimmune disorder (such as rheumatoid arthritis, Bechet's disease, ankylosing spondylitis, sarcoidosis, and the like), as an isolated immune mediated ocular disorder (such as pars planitis or iridocyclitis, and the like), as a disease unassociated with known etiologies, and following certain systemic diseases which cause antibody-antigen complexes to be deposited in the uveal tissues.

Illustratively, the conjugates described herein administered to kill, eliminate or reduce in number inflammatory cells or suppress their function can be administered parenterally to the patient suffering from the disease state, for example, intradermally, subcutaneously, intramuscularly, intraperitoneally, or intravenously in combination with a pharmaceutically acceptable carrier. In another embodiment, the conjugates described herein can be administered to the patient by other medically useful procedures and effective doses can be administered in standard or prolonged release dosage forms. In another aspect, the therapeutic methods described herein can be used alone or in combination with other therapeutic methods recognized for treatment of inflammation.

In some embodiments, pharmaceutical compositions comprising an amount of a conjugate effective to eliminate, reduce in number, kill or suppress the function of a population of pathogenic cells, such as inflammatory cells, in a patient when administered in one or more doses are described. In such embodiments, the conjugate can be administered to the patient parenterally, e.g., intradermally, subcutaneously, intramuscularly, intraperitoneally, intravenously, or intrathecally. Alternatively, the conjugate can be administered to the patient by other medically useful processes, such as orally, and any effective dose and suitable therapeutic dosage form, including prolonged release dosage forms, can be used.

For example, the conjugates and compositions described herein may be administered orally. Oral administration may involve swallowing, so that the conjugate or composition enters the gastrointestinal tract, or buccal or sublingual administration may be employed by which the conjugate or composition enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The conjugates and compositions described herein may also be used in fast-dissolving, fast-disintegrating dosage forms such as those described in Expert Opinion in Therapeutic Patents, 11 (6), 981-986, by Liang and Chen (2001). For tablet dosage forms, depending on dose, the conjugate may make up from 1 weight % to 80 weight % of the dosage form, more typically from 5 weight % to 60 weight % of the dosage form. In addition to the conjugates and compositions described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a conjugate as described herein, a film-forming polymer, a binder, a solvent, a humectant, a plasticizer, a stabilizer or emulsifier, a viscosity-modifying agent and a solvent. Some components of the formulation may perform more than one function.

Solid formulations for oral administration may be formulated to be immediate and/or modified release formulations. Modified release formulations include delayed-, sustained-, pulsed-, controlled-, targeted and programmed release formulations. Suitable modified release formulations for the purposes of the disclosure are described in U.S. Pat. No. 6,106,864.

Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The conjugates described herein can also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous.

Suitable devices for parenteral administration include needle (including micro-needle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art. The solubility of conjugates described herein used in the preparation of parenteral solutions may be increased by the use of appropriate formulation techniques, such as the incorporation of solubility-enhancing agents.

Formulations for parenteral administration may be formulated to be immediate and/or modified release formulations. Modified release formulations include delayed-, sustained-, pulsed-, controlled-, targeted and programmed release formulations. Thus conjugates described herein can be formulated as a solid, semi-solid, or thixotropic liquid for administration as an implanted depot providing modified release of the active compound. Examples of such formulations include drug-coated stents and poly(lactic-coglycolic)acid (PGLA) microspheres. The conjugates described herein can also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm Sci, 88 (10), 955-958 by Finnin and Morgan (October 1999). Other means of topical administration include delivery by electroporation, iontophoresis, phonophoresis, sonophoresis and microneedle or needle-free (e.g. Powderject™, Bioject™ etc.) injection.

Examples of parenteral dosage forms include aqueous solutions of the conjugates described herein, in an isotonic saline, 5% glucose or other well-known pharmaceutically acceptable liquid carriers such as liquid alcohols, glycols, esters, and amides. The parenteral dosage form can be in the form of a reconstitutable lyophilizate comprising the dose of the conjugate. In one aspect of the present embodiment, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

In one illustrative aspect, at least one additional composition comprising a therapeutic factor can be administered to the host in combination or as an adjuvant to enhance the conjugate-mediated elimination of the population of pathogenic cells, such as inflammatory cells, or more than one additional therapeutic factor can be administered. The therapeutic factor can be selected from an agent, or another therapeutic factor capable of complementing the efficacy of the administered conjugate.

In one illustrative aspect, therapeutically effective combinations of these factors can be used. For example, therapeutically effective amounts of the therapeutic factor, for example, in amounts ranging from about 0.1 MIU/m²/dose/day to about 15 MIU/m²/dose/day in a multiple dose daily regimen, or for example, in amounts ranging from about 0.1 MIU/m²/dose/day to about 7.5 MIU/m²/dose/day in a multiple dose daily regimen, can be used along with the conjugates described herein to eliminate, reduce, suppress the function of or neutralize pathogenic cells, such as inflammatory cells, in a patient harboring the pathogenic cells (MIU=million international units; m²=approximate body surface area of an average human).

In another illustrative aspect, any effective regimen for administering the conjugates can be used. For example, the conjugates can be administered as single doses, or can be divided and administered as a multiple-dose daily regimen. In other embodiments, a staggered regimen, for example, one to three days per week can be used as an alternative to daily treatment, and such intermittent or staggered daily regimen is considered to be equivalent to every day treatment and within the scope of the methods described herein. In one embodiment, the patient is treated with multiple injections of the conjugate to eliminate the population of pathogenic cells, such as inflammatory cells. In another embodiment, the patient is injected multiple times (preferably about 2 up to about 50 times) with the conjugate, for example, at 12-72 hour intervals or at 48-72 hour intervals. In other embodiments, additional injections of the conjugate can be administered to the patient at an interval of days or months after the initial injections(s) and the additional injections prevent recurrence of the disease state caused by the pathogenic cells, such as inflammatory cells.

Formulations for topical administration may be formulated to be immediate and/or modified release formulations. Modified release formulations include delayed-, sustained-, pulsed-, controlled-, targeted and programmed release formulations. The conjugates described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. The pressurized container, pump, spray, atomizer, or nebulizer contains a solution or suspension of the conjugates(s) of the present disclosure comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the conjugate is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules (made, for example, from gelatin or hydroxypropylmethylcellulose), blisters and cartridges for use in an inhaler or insufflator may be formulated to contain a powder mix of the conjugate described herein, a suitable powder base such as lactose or starch and a performance modifier such as Iso-leucine, mannitol, or magnesium stearate.

The lactose may be anhydrous or in the form of the monohydrate, preferably the latter. Other suitable excipients include dextran, glucose, maltose, sorbitol, xylitol, fructose, sucrose and trehalose. A typical formulation may comprise a conjugate of the present disclosure, propylene glycol, sterile water, ethanol and sodium chloride. Alternative solvents which may be used instead of propylene glycol include glycerol and polyethylene glycol.

The conjugates described here can be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of conjugates together with one or more additional compounds, for example, for the purpose of treating a particular disease or condition, it is within the scope of the present disclosure that two or more pharmaceutical compositions, at least one of which contains a conjugate as described herein, may conveniently be combined in the form of a kit suitable for co-administration of the compositions. Thus the kit of the present disclosure comprises two or more separate pharmaceutical compositions, at least one of which contains a conjugate as described herein, and means for separately retaining said compositions, such as a container, divided bottle, or divided foil packet. An example of such a kit is the familiar blister pack used for the packaging of tablets, capsules and the like. The kit of the present disclosure is particularly suitable for administering different dosage forms, for example parenteral, for administering the separate compositions at different dosage intervals, or for titrating the separate compositions against one another. To assist compliance, the kit typically comprises directions for administration and may be provided with a so-called memory aid.

The disclosure includes all pharmaceutically acceptable isotopically-labelled conjugates, and their drug incorporated therein, wherein one or more atoms are replaced by atoms having the same atomic number, but an atomic mass or mass number different from the atomic mass or mass number which predominates in nature.

Examples of isotopes suitable for inclusion in the conjugates, and their drug incorporated therein, include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled conjugates, and their drug incorporated therein, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, and ¹³N, can be useful in Positron Emission Topography (PET) studies for examining substrate receptor occupancy. Isotopically-labeled conjugates, and their Drug(s) incorporated therein, can generally be prepared by conventional techniques known to those skilled in the art or by processes analogous to those described in the accompanying Examples using an appropriate isotopically-labeled reagents in place of the non-labeled reagent previously employed.

It will be understood that the conjugates described herein, and their constituent parts B and D¹ can exist in different tautomeric forms. As used herein, the term “tautomer” has its ordinary meaning known to one of skill in the art. That being constitutional isomers of organic compounds that readily interconvert by a chemical reaction called tautomerization. It will be readily appreciated by one of skill in the art that because of rapid interconversion, tautomers can generally be considered to be the same chemical compound. Examples of tautomers include but are not limited to enol-keto tautomers, amine-imine tutomers, and the like.

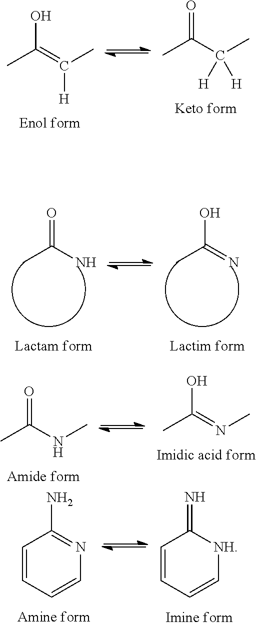

### EXAMPLES

### Chemistry Examples

Materials. N¹⁰-trifluoroacetylpteroic acid can be purchased from Irvine Chemistry Lab (Anaheim, Calif.) and can also be prepared according to Xu et al., U.S. Pat. No. 8,044,200. EC0475 can be prepared according to Leamon et al., U.S. patent application Ser. No. 13/841,349, filed on Mar. 5, 2013. Aminopteroic acid can be purchased from Cambridge Major Laboratories (Germantown, Wis.). Peptide synthesis reagents, H-L-Glu(OMe)-O-t-Bu-HCl, Fmoc-L-Glu-(O-t-Bu)-OH, PyBOP and Boc-S-3-nitro-2-pyridinesulfenyl-L-cysteine (Boc-NPS-Cys)_can be purchased from Chem-Impex International (Wood Dale, Ill.). 2-Chlorotrityl Chloride polymer resin and Fmoc-S-Trityl-L-pencillamine can be purchased from AAPPTec (Louisville, Ky.). N,N-Dimethylformamide (DMF), MeOH, Acetonitrile, Isopropanol (IPA), Piperidine, Triethylamine (TEA), N,N-Diisopropylethlamine (DIPEA), Trifluoroacetic acid (TFA), Triisopropylsilane (TIPS), Toluene, N-methyl 2-pyrollidone (NMP) can be purchased from Sigma-Aldrich (St. Louis. Mo.).

### Example 1: Synthesis of EC2452

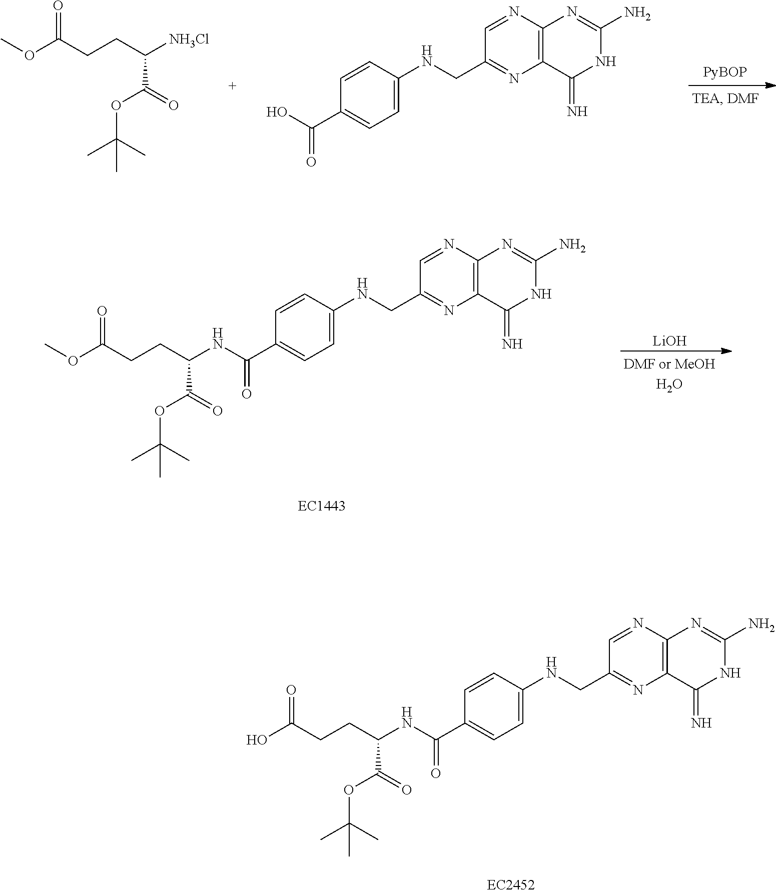

Aminopteroic acid (12 g, 38.6 mmol), H-L-Glu(OMe)-O-t-Bu HCl salt (10.8 g, 42.5 mmol, 1.15 equiv.), and PyBOP (30 g, 57.6 mmol, 1.5 equiv.) were suspended in 200 mL DMF. To the suspension, TEA (19.5 mL, 140 mmol, 3.6 equiv.) was added. After 1 hr, LC/MS showed complete conversion. The reaction mixture was poured into 900 mL H₂O, and then filtered through a Buchner funnel with Whatman grade 1 filter paper. The filter cake was washed with another 900 mL H₂O. The damp crude solid was transferred into a bottle, frozen and placed on the freeze dryer several days to give 20 g of crude product EC1443.

Aminopterin diester EC1443 (10 g, ca. 19.5 mmol) was suspended 30 mL DMF and 30 mL of H₂O. A solution of LiOH—H₂O (1.6 g, 38.1 mmol, 2 equiv.) in a minimum amount of H₂O was added to the aminopterin diester suspension solution. After 30 minutes, the reaction mixture became clear and LC/MS showed complete conversion. Majority of DMF was removed by diethyl ether extraction. Then the pH of the aqueous solution was adjusted to about 9 with dilute HCl. The solution was loaded onto 30 g Biotage C18 column directly and purified with H₂O/acetonitrile to afford 3 g of EC2452 as yellow solid after lyophilization.

LC/MS conditions: 10 to 100% acetonitrile, 20 mM NH₄HCO₃ buffer (pH=7). LC/MS (ESI) 497.47 [M+H]⁺

EC2452 ¹H-NMR (500 MHz, DMSO-d6): 8.68 (s, 1H), 7.68 (d, J=8.8 Hz, 2H), 6.71 (d, J=8.8 Hz, 2H), 3.98 (t, J=6.3 Hz, 1H), 2.05 (m, 2H), 1.84 (m, 2H), 1.35 (s, 9H).

### Example 2: Synthesis of EC0804

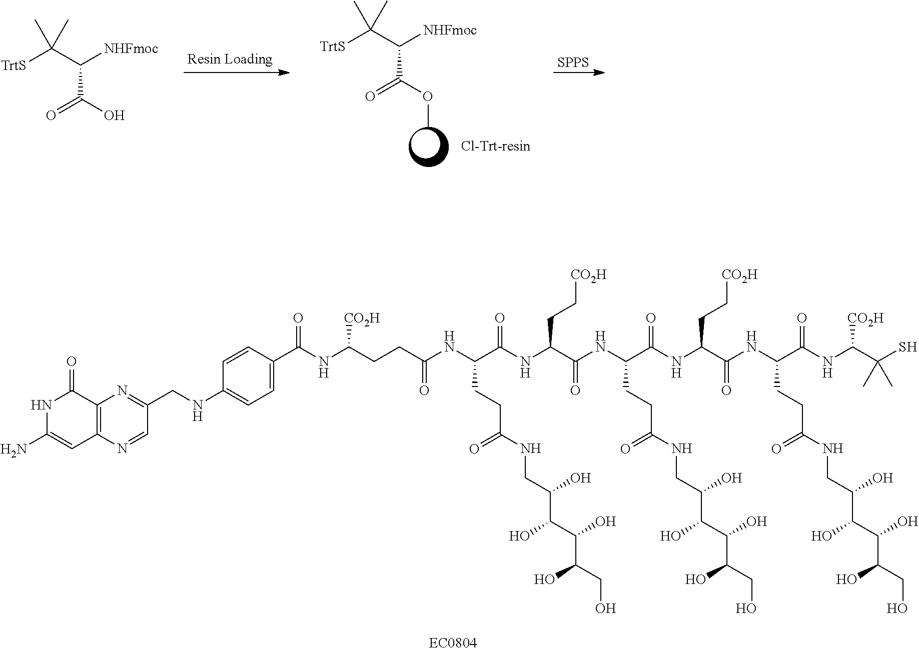

Commercially available 2-Chlorotrityl Chloride polymer resin (9.80 g, 11.0 mmol, 1.12 mmol/g, 100-200 mesh) was placed within a solid-phase vessel to which anhydrous dichloromethane (140 mL) was added. The solution was purged with argon and Fmoc-S-Trityl-L-pencillamine (6.69 g, 11.0 mmol, 1 eq.) dissolved in anyhydrous dimethylformamide (140 mL) together with N, N-Diisopropylethlamine (7.70 mL, 44.0 mmol, 4 eq.) added. After 1 h. MeOH (70 mL) was added to the reaction mixture and the vessel drained of all solvent. The remaining resin beads were washed consecutively with MeOH (3×70 mL), DMF (3×70 mL) and IPA (3×70 ml) before drying overnight under high vacuum to yield 12.20 g loaded resin.

The loaded volume of Fmoc-S-Trityl-L-pencillamine bound resin (mmol/g) was determined as follows. Three vials containing commercially available Fmoc-S-Trityl-L-pencillamine (10.32 mg, 6.23 mg, 2.40 mg) were prepared along with another three vials containing the loaded resin (20.78 mg, 20.58 mg, 20.38 mg). Each vial was treated with a 20% piperidine/dimethylformamide solution (1.0 mL) and the reaction mixtures stirred for 1 h. The contents of each vial were transferred to six, 50 mL volumetric flasks respectively and each vial washed in turn with HPLC grade MeOH (5×5 mL). The remaining volume of each flask was filled with HPLC grade MeOH and the contents mixed thoroughly. The absorbance of each solution was then measured using a M200 UV spectrophotometer relative to a methanol blank. The data for the three solutions containing deprotected Fmoc-S-Trityl-L-pencillamine were used to generate a standard curve of Absorbance versus Mass of Fmoc-S-Trityl-L-pencillamine (mg). A trend line was fitted with equation y=0.0894x−0.0011. This in turn was used to determine the loaded volume of Fmoc-S-Trityl-L-pencillamine bound resin (mmol/g), calculated to be an average of 0.32 mmol/g such that the loaded resin (12.20 g, 3.90 mmol, 0.32 mmol/g) was obtained in a 36% yield.

Penicillamine-2-Cl-trityl resin was subjected to the standard Fmoc solid phase peptide synthesis conditions to afford EC0804 with about 50% yield and 97% purity after Biotage C18 column purification with 0.1% TFA (0% to 25% to 35% to 50%).

### Exemplary Synthesis of EC0804

| | | | MW | Amount |
|---|---|---|---|---|
| Reagents | mmol | equivalent | (g/mol) | (g) |
| Fmoc-L-Pen(trityl)-2- | 4.05 | | | 7.25 |
| chlorotrityl- Resin | | | | |
| (loading 0.56 mmol/g) | | | | |
| EC0475 | 8.1 | 2 | 612.67 | 5.0 |
| Fmoc-Glu(OtBu)—OH | 8.1 | 2 | 425.47 | 3.4 |
| EC0475 | 6.48 | 1.6 | 612.67 | 3.9 |
| Fmoc-Glu(OtBu)—OH | 8.1 | 2 | 425.47 | 3.4 |
| EC0475 | 6.48 | 1.6 | 612.67 | 3.9 |
| Fmoc-Glu-OtBu | 8.1 | 2 | 425.47 | 3.4 |
| N¹⁰—TFA-Pteroic Acid | 7.1 | 1.8 | 408.29 | 2.9 |
| (dissolve in 10 ml DMSO) | | | | |
| DIPEA | | 2.0X eq of AA | | |
| PyBOP | | 1.0X eq of AA | | |

The resin was added to a peptide synthesis vessel and then the resin was swelled with DMF for 10 min. Before each amino acid coupling step, the resin was treated with 20% piperidine in DMF for Fmoc deprotection (3×10 min) and subsequently washed with 3×DMF, IPA, and DMF again. The Fmoc deprotection via piperidine treatment was repeated a second time to ensure complete Fmoc deprotection. For each coupling step, the appropriate amino acid, DMF, DIPEA, and PyBOP were added to the reactor. The reaction mixture was agitated with argon bubbling (overnight for the first EC0475 coupling and 1 hr. for all of the other coupling steps) and washed 3× with DMF, IPA, and DMF again. Continue to complete all 7 coupling steps. The peptide was then cleaved from the resin by treatment of the resin with a TFA/H₂O/TIPS/EDT (92.5:2.5:2.5:2.5) cleavage solution with argon bubbling for 1 hr. The cleavage solution was then poured into diethyl ether to affect precipitation of crude peptide. After isolation of the solid by filtration or centrifugation, the crude peptide was treated with aqueous sodium carbonate (pH=10) under argon bubbling for 1 hr. to cleave the TFA protecting group. After purification and desalting, pure EC0804 (>98% purity, 2.7 g, 40% yield) was obtained.

LC/MS conditions: 5 to 50% acetonitrile, 0.1% formic acid. LC/MS (ESI) 854.93 [M+2H]²⁺

EC0804 ¹H-NMR (500 MHz, D₂O): 8.62 (s, 1H), 7.51 (d, J=7.5 Hz, 2H), 6.64 (d, J=7.5 Hz, 2H), 4.51 (s, 2H), 4.35-4.33 (m, 1H), 4.31-4.29 (m, 2H), 4.26-4.23 (m, 1H), 4.15-4.07 (m, 3H), 3.77-3.71 (m, 3H), 3.71-3.68 (m, 1H), 3.66-3.60 (m, 6H), 3.56-3.49 (m, 6H), 3.33-3.24 (m, 3H), 3.16-3.09 (m, 3H), 2.46-2.36 (m, 3H), 2.36-2.14 (m, 11H), 2.04-1.72 (m, 12H), 1.35 (s, 3H), 1.27 (s, 3H).

### Example 3: Synthesis of EC2317

### Steps 1 and 2

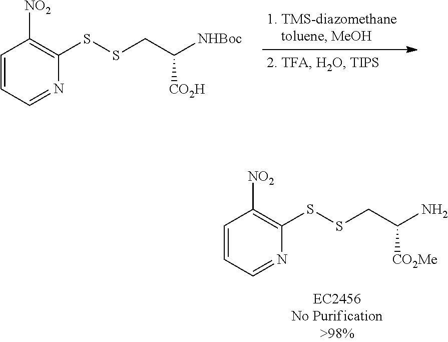 [SMILES: CC(C)(C)OC(=O)N[C@@H](CSSc1ncccc1[N+](=O)[O-])C(=O)O ; COC(=O)[C@@H](N)CSSc1ncccc1[N+](=O)[O-]]

Boc-Cys(Npys)-OH (3.81 g, 10.2 mmol) was dissolved in toluene (45 mL) and MeOH (45 mL). To this solution, at room temperature, with stirring was added a solution of TMS-diazomethane in diethyl ether (9 mL of a 2M solution, 1.8 eq.), dropwise. After 10 min, TLC (5% MeOH in DCM) showed complete conversion. The solvent and excess reagent was then removed under reduced pressure and dried under the high vacuum for several hours to yield about 4 g of crude material. The material was carried to the next reaction without further purification.

EC2456 ¹H-NMR (500 MHz, CD₂Cl₂, crude product of methylation): 8.94 (br, 1H), 8.54 (dd, 1H), 7.43 (d, 1H), 6.39 (br, 1H), 4.55 (br, 1H), 3.70 (s, 3H), 3.47 (dd, 1H), 3.26 (dd, 1H), 1.45 (s, 9H).

Boc deprotection was accomplished with the standard TFA/H₂O/TIPS cleavage solution (95:2.5:2.5). 1.3 g of the methyl ester was treated with the cleavage solution (12 mL) for 45 min. UPLC showed the reaction was complete. The cleavage solution was removed under reduced pressure and the resulting residue was placed on the high vacuum for at least 2 hours. This material (EC2456) was used in the next reaction without further purification. LC/MS (ESI) 290.24 [M+H]⁺.

### Steps 3 and 4

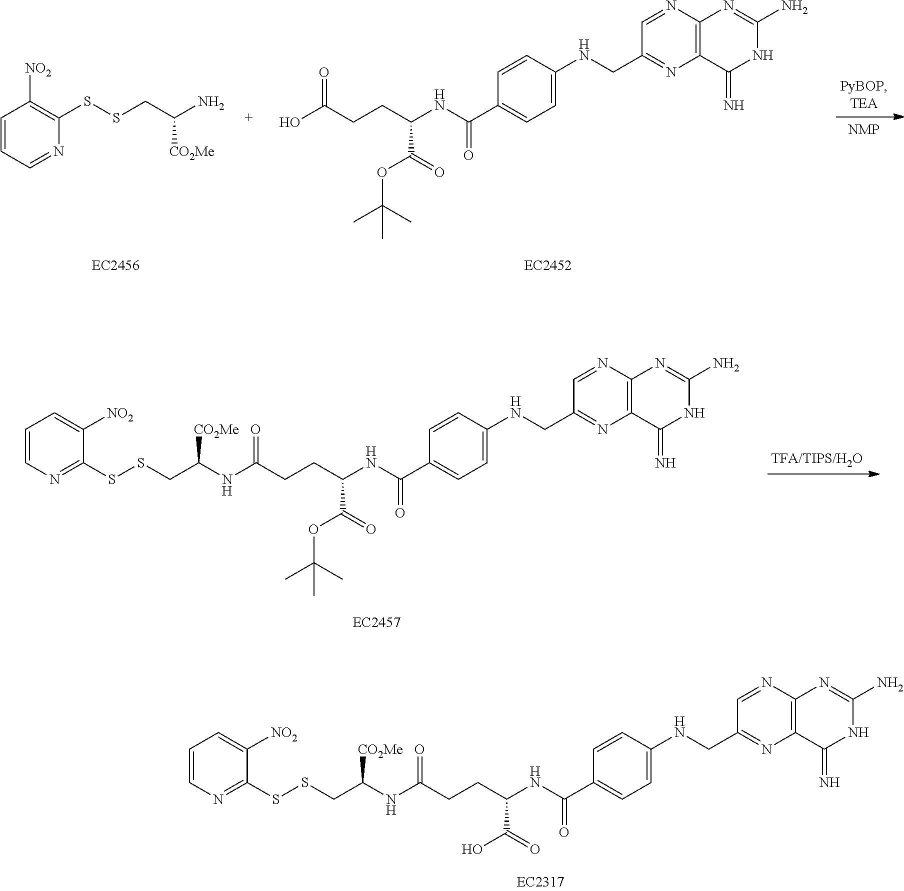 [SMILES: COC(=O)[C@@H](N)CSSc1ncccc1[N+](=O)[O-] ; CC(C)(C)OC(=O)[C@H](CCC(=O)O)NC(=O)c1ccc(NCc2cnc3nc(N)[nH]c(=N)c3n2)cc1 ; COC(=O)[C@H](CSSc1ncccc1[N+](=O)[O-])NC(=O)CC[C@H](NC(=O)c1ccc(NCc2cnc3nc(N)[nH]c(=N)c3n2)cc1)C(=O)OC(C)(C)C ; COC(=O)[C@H](CSSc1ncccc1[N+](=O)[O-])NC(=O)CC[C@H](NC(=O)c1ccc(NCc2cnc3nc(N)[nH]c(=N)c3n2)cc1)C(=O)O]

Aminopterin α-t-butyl ester EC2452 (1.53 g, 3.08 mmol) was suspended in NMP (30 mL). To this suspension was added TEA (2.36 mL, 5.5 eq.), PyBOP (3.5 g, 2.2 eq.), and NPS-Cys-OMe EC2456 (crude residue from reaction above from 1.3 g of Boc protected precursor, re-constituted in 5 mL NMP, 1.1 eq.). The reaction mixture became clear. After 45 minutes, UPLC showed the reaction to be complete. The reaction mixture was precipitated with 900 mL cold Et₂O. The precipitate was recovered by centrifugation/removal of the solvent. The solid was washed with H₂O (2×) and separated by centrifugation/removal of solvent. The crude product containing EC2457 was used without further purification. LC/MS (ESI) 768.70 [M+H]⁺.

The crude product containing EC2457 was dissolved in 12 mL TFA/TIPS/H₂O (95:2.5:2.5) and stirred at room temperature. LC/MS was used to monitor the reaction. After the reaction was complete, the reaction mixture was precipitated with cold Et₂O. The precipitate was recovered by centrifugation/removal of the solvent. The solid was washed Et₂O and separated by centrifugation/removal of the solvent. The solid was dried under vacuum for 2 hr to give around 3 g of crude yellow solid. The crude product containing EC2317 was then dissolved in DMSO (6 mL) and purified by Biotage C18 column (ammonium bicarbonate (pH 7) and acetonitrile as elutents) to give 1.6 g of purified EC2317 (68% yield over two steps, 90-95% purity) as well as 175 mg of partially purified material (85% purity).

LC/MS conditions: 10 to 100% acetonitrile, 20 mM NH₄HCO₃ buffer (pH=7).

LC/MS (ESI) 712.45 [M+H]⁺

EC2317 ¹H-NMR (500 MHz, CD₃OD): 8.80 (dd, J=4.8, 1.8 Hz, 1H), 8.68 (s, 1H), 8.60 (dd, J=8.2, 1.5 Hz, 1H), 7.65 (d, J=8.8 Hz, 2H), 7.55 (dd, J=8.4, 4.8 Hz, 1H), 6.71 (d, J=8.8 Hz, 2H), 4.56 (dd, J=8.6, 5.2 Hz, 1H), 3.98 (t, J=6.3 Hz, 1H), 3.55 (s, 3H), 3.20 (dd, J=13.9, 5.2 Hz, 1H), 3.07 (dd, J=13.7, 8.8 Hz, 1H), 2.22 (t, J=2.0 Hz, 2H), 2.02 (m, 1H), 1.88 (m, 1H).

### Example 4: Synthesis of EC2319

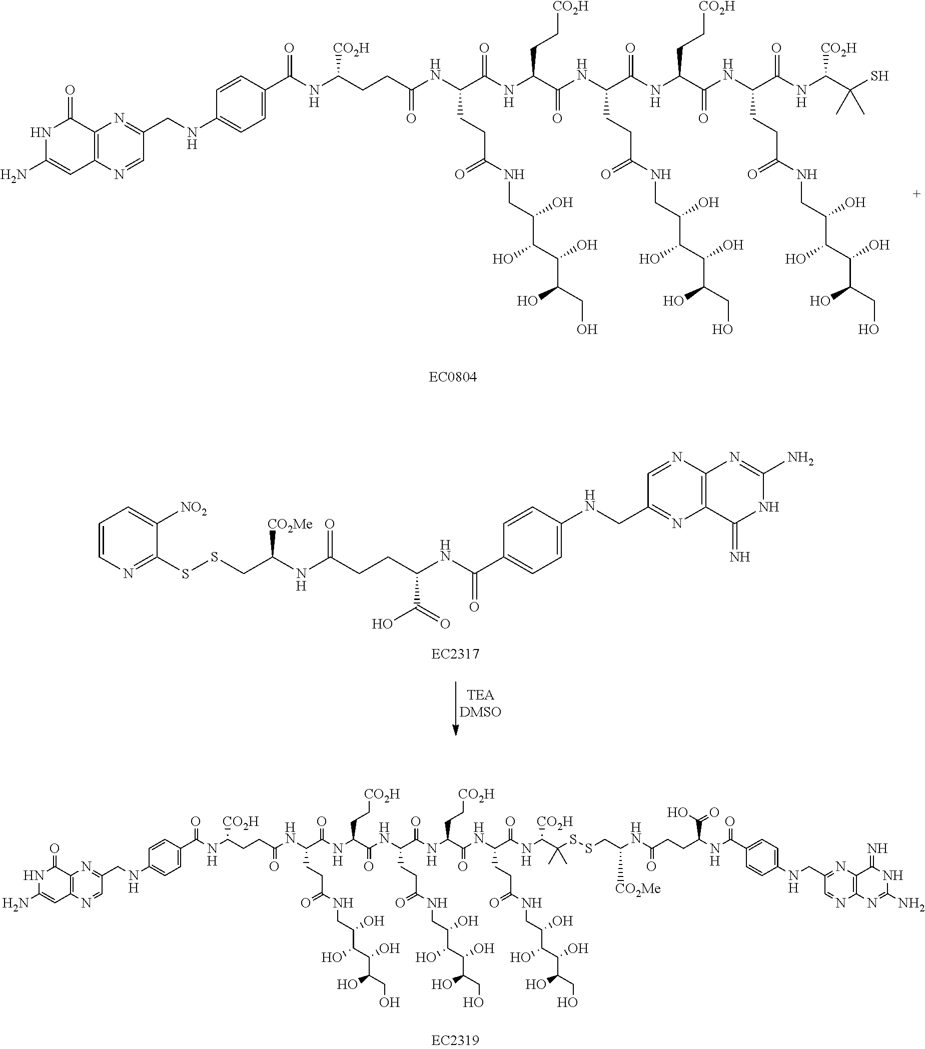

EC0804 (1.67 g, 0.98 mmol) was dissolved in DMSO (15 mL) and purged with argon for 10 min. To this solution was added TEA (1.37 mL, 10 eq.) followed by NPS-Cys-OMe-AMT EC2317 (700 mg, 1 eq.) in DMSO (5 mL). The solution was allowed to stir for 20 min with continued argon bubbling. UPLC showed the reaction was complete. The reaction mixture was poured into 200 mL of cold H₂O with stirring and then purified via a 400 g Biotage C18 column (NH₄HCO₃ buffer (pH=7)/acetonitrile as the eluents). Fractions of greater than 98% purity were collected. Fractions of moderate purity were collected and re-purified as practical. After freeze drying, pure EC2319 (>98%) was recovered as a yellow solid (1.4 g, 64% yield). LC/MS conditions: 0 to 30% acetonitrile, 20 mM NH₄HCO₃ buffer (pH=7).

LC/MS (ESI) 1133.46 [M+2H]²⁺

EC2319 ¹H-NMR (500 MHz, D₂O): 8.63 (s, 1H), 8.57 (s, 1H), 7.53 (dd, 4H), 6.65 (d, J=8.8 Hz, 2H), 6.59 (d, J=8.8 Hz, 2H), 4.45 (br, 4H), 4.35 (s, 2H), 4.19 (m, 2H), 4.16-4.07 (m, 7H), 4.07-4.01 (m, 2H), 3.65-3.46 (m, 15H), 3.42-3.35 (m, 6H), 3.26-3.14 (m, 3H), 3.08-2.92 (m, 4H), 2.96-2.88 (m, 1H), 2.20-2.00 (m, 14H), 2.00-1.70 (m, 14H), 1.22 (s, 3H), 1.14 (s, 3H).

### Example 5: Synthesis of EC2413

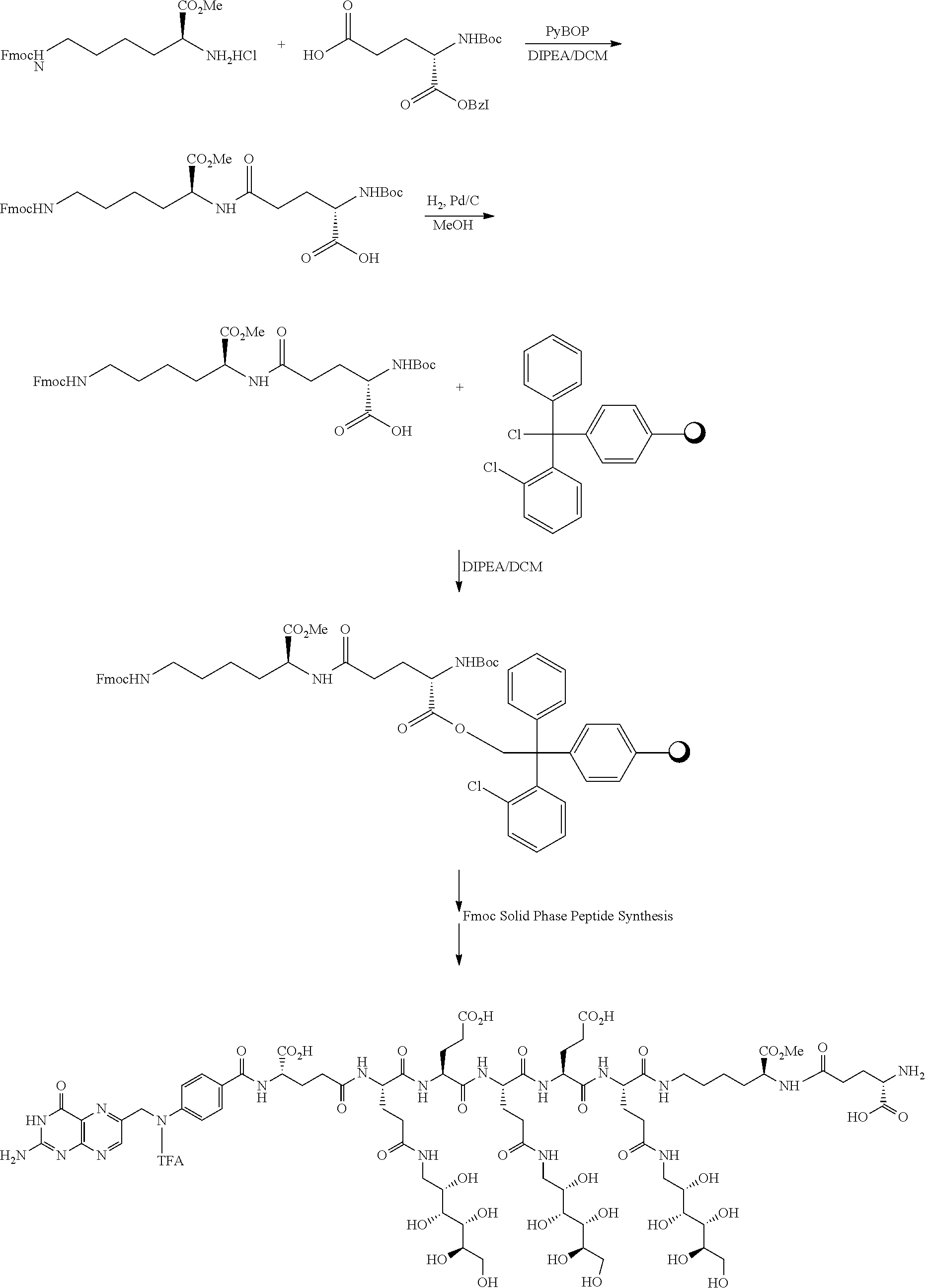

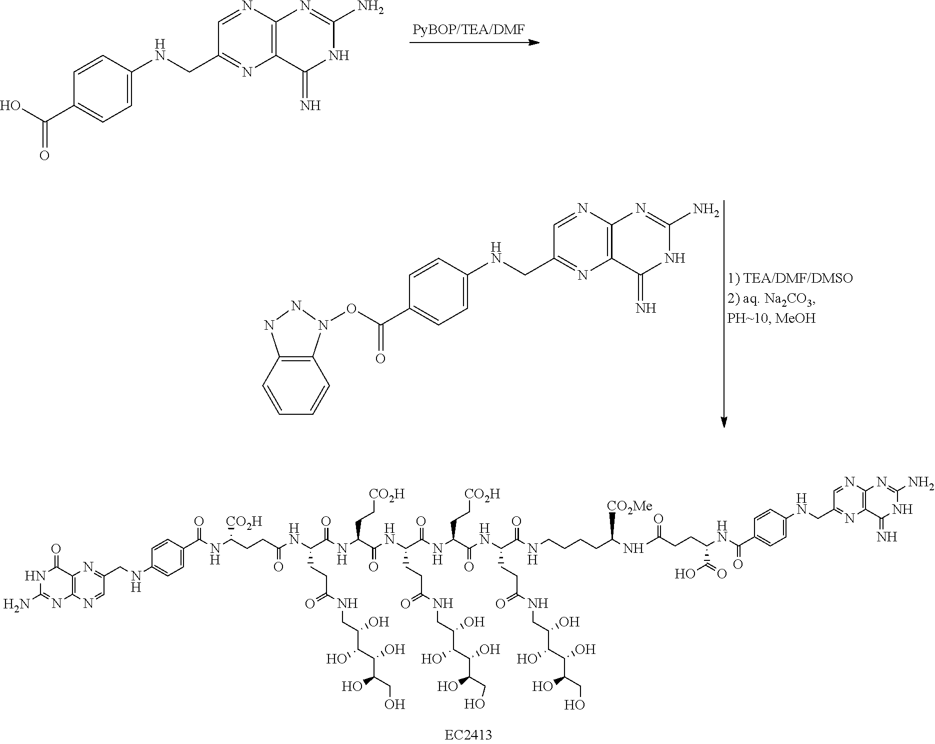 [SMILES: N=c1[nH]c(N)nc2ncc(CNc3ccc(C(=O)O)cc3)nc12 ; N=c1[nH]c(N)nc2ncc(CNc3ccc(C(=O)ON4NNc5ccccc54)cc3)nc12 ; COC(=O)[C@H](CCCCNC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CC[C@H](NC(=O)c1ccc(NCc2cnc3nc(N)[nH]c(=O)c3n2)cc1)C(=O)O)NC(=O)CC[C@H](NC(=O)c1ccc(NCc2cnc3nc(N)[nH]c(=N)c3n2)cc1)C(=O)O]

### Step 1: Synthesis of Boc-Glu-[Lys(Fmoc)-OMe]-OBzl

H-Lys(Fmoc)-OMe HCl salt (2.50 g, 5.97 mmol) was dissolved in dichloromethane (˜10 mL). To this solution was added Boc-Glu-OBzl (2.21 g, 1.1 eq.), PyBOP(4.65 g, 1.5 eq.) and DIPEA (3.1 mL, 3 eq.). This solution was allowed to stir for 30 minutes. LC/MS was used to monitor the reaction. After the reaction was completed, the reaction mixture was loaded directly onto a silica column and purified with DCM/MeOH as eluents. 4.90 g of the product was recovered. ¹H-NMR (500 MHz, CD3OD): 7.78 (d, J=7.8 Hz, 2H), 7.64 (d, J=7.3 Hz, 2H), 7.40-7.26 (m, 9H), 5.16 (d, J=12.2 Hz, 1H), 5.10 (d, J=12.2 Hz, 1H), 4.34 (m, 3H), 4.18 (t, J=6.8 Hz, 1H), 4.14 (m, 1H), 3.68 (s, 3H), 3.05 (t, br, 2H), 2.33 (t, J=7.6 Hz, 2H), 2.15 (m, 1H), 1.80-1.75 (m, 2H), 1.72-1.63 (m, 1H), 1.51-1.45 (m, 2H), 1.41 (s, 9H), 1.38-1.30 (m, 3H).

### Step 2: Synthesis of Boc-Glu-[Lys(Fmoc)-OMe]-OH

Boc-Glu-[Lys(Fmoc)-OMe]-OBzl (2.74 g, 3.91 mmol) from Step 1 was dissolved in anhydrous MeOH (120 mL). To the solution was added 10% Pd/C (192 mg, 0.18 mmol), and the reaction was stirred at room temperature under H₂ (balloon). After 20 minutes, LC/MS showed the reaction was completed. The catalyst was removed by filtration through celite. The filtrate was concentrated under reduced pressure. The residue was purified on a silica column using DCM/MeOH as eluents to yield 1.70 g of Boc-Glu-[Lys(Fmoc)-OMe]-OH (71% yield).

¹H-NMR (500 MHz, CD3OD): 7.79 (d, J=7.4 Hz, 2H), 7.64 (d, J=7.4 Hz, 2H), 7.37 (t, J=7.6 Hz, 2H), 7.30 (t, J=7.3 Hz, 2H), 4.34 (m, 3H), 4.19 (t, J=6.9 Hz, 1H), 4.08 (m, 1H), 3.69 (s, 3H), 3.09 (t, J=6.1 Hz, 2H), 2.35 (t, J=7.8 Hz, 2H), 2.15 (m, 1H), 1.94-1.86 (m, 3H), 1.74-1.63 (m, 1H), 1.54-1.46 (m, 2H), 1.40 (s, 9H), 1.30 range overlapped with DIPEA impurity.

### Step 3: Loading of the Chlorotrityl Resin

The 2-chlorotrityl chloride resin (0.958 g, 0.978 mmol, resin loading is 1.02 mmol/g) was placed in the solid phase vessel. Boc-Glu-[Lys(Fmoc)-OMe]-OH from Step 2 (599 mg, 0.978 mmol) was dissolved in 10 mL of anhydrous DCM. DIPEA (850 μL, 5 eq.) was added to the dipeptide solution, and this solution was added to the resin with argon purging. After 5 minutes, an additional 255 μL of DIPEA (1.5 eq.) was added. The reaction mixture was purged with argon for 1 hour. MeOH (5 mL) was added and the reaction mixture was purged with argon for 15 minutes. The solution was drained and the resin was washed with DMF, IPA and MeOH. The resin was dried under vacuum. The weight of resin had increased by 30 mg, and the loading was estimated to be 0.45 mmol/g.

### Step 4: Synthesis of the Folate Spacer-Linker Unit

222 mg of loaded resin from Step 3 (0.10 mmol) was coupled to amino acids using standard Fmoc solid phase peptide synthesis methodology with PyBOP (104 mg for every amino acid coupling step, 0.20 mmol) as the coupling reagent. The amino acid sequence is EC0475 (123 mg, 0.200 mmol), Fmoc-Glu(O-t-Bu)-OH (85 mg, 0.20 mmol), EC0475 (123 mg, 0.200 mmol), Fmoc-Glu(O-t-Bu)-OH (85 mg, 0.20 mmol), EC0475 (123 mg, 0.200 mmol), Fmoc-Glu-O-t-Bu (85 mg, 0.20 mmol), and N¹⁰-TFA-Pteroic acid (105 mg, 0.250 mmol). The Folate spacer-linker unit was cleaved from the resin using a TFA/TIPS/H₂O (95:2.5:2.5) solution with 5 eq. of EDT and was purified on a C18 column with 0.1% TFA aqueous solution and acetonitrile as eluents. After removing acetonitrile, the aqueous solution was frozen and lyophilized to afford 113 mg of the product (58% yield). LC/MS (ESI) 973.32 [M+2H]²⁺ Selected signals: ¹H-NMR (500 MHz, DMSO-d6): 8.57 (s, 1H), 7.86 (d, J=8.3 Hz, 2H), 7.56 (d, J=8.3 Hz, 2H), 5.08 (s, 2H), 4.35-4.28 (m, 1H), 4.20-4.10 (m, 5H), 4.10-4.04 (m, 1H), 3.87 (t, 1H), 3.62-3.55 (m, 5H), 3.54 (d, 1H), 3.53-3.50 (m, 4H), 3.47-3.42 (m, 3H), 3.40-3.32 (m, 6H), 3.26-3.18 (m, 3H), 3.08-2.90 (m, 5H), 2.40-2.20 (m, 9H), 2.15-2.03 (m, 7H), 2.03-1.90 (m, 3H), 1.90-1.78 (m, 6H), 1.78-1.50 (m, 8H), 1.38-1.30 (m, 2H), 1.26-1.16 (m, 2H).

### Step 5: Synthesis of Aminopterin HOBt Activated Acid

Amino-pteroyl HOBt active ester was prepared by allowing 120 mg (385 μmol) of amino-pteroic acid to react with 241 mg (463 μmol) of PyBOP in the presence of 0.19 mL (1.4 mmol) of triethylamine and 2.7 mL of DMF. After 1 hour, the reaction mixture was filtered to remove solids. Upon standing, additional solids precipitated from the filtrate. The second crop of precipitate was collected by filtration, and the second crop was washed with ethyl acetate. Both crops of solids were dried under vacuum. The dried solids weighed 67 mg (first crop) and 72 mg (second crop). HPLC analysis revealed that the first crop had 77.8% peak area purity, and the second crop had 93.0% peak area purity.

### Step 6: Synthesis of EC2413

Amino pteroic acid HOBt activated ester from Step 5 (57.4 mg, 1.6 eq.) was suspended in DMF (1 mL), DMSO (1.8 mL), and TEA (112 μL, 10 eq.). To this mixture was added the peptide from Step 4 (154 mg, 0.079 mmol) in DMF (1.5 mL) and DMSO (300 μL). The reaction was allowed to stir at room temperature overnight. The reaction was poured into 0.1M phosphate buffer (pH=7.3). This solution was loaded onto a Biotage C18 column and purified (20 mM ammonium bicarbonate/acetonitrile eluents). After freeze-drying, the residue was dissolved in water/MeOH (2 mL/2 mL) and 5% sodium carbonate was added to increase the pH to 10. The reaction was stirred for 90 minutes. The MeOH was removed under reduced pressure after the aqueous solution had been adjusted to neutral pH upon addition of acetic acid. The solution was diluted with water and loaded onto a Biotage C18 column (20 mM ammonium bicarbonate/acetonitrile eluents) and purified to give EC2413 as a yellow solid (64 mg, 38% yield). LC/MS (ESI) 1071.82 [M+2H]²⁺ Selected signals: ¹H-NMR (500 MHz, DMSO-d6): 8.64 (s, 1H), 8.59 (s, 1H), 7.55 (dd, 4H), 6.66 (d, J=8.8 Hz, 2H), 6.60 (d, J=8.8 Hz, 2H), 4.46 (br, 4H), 4.10-4.00 (m, 7H), 3.65-3.57 (m, 3H), 3.56-3.51 (m, 6H), 3.50 (s, 3H), 3.49-3.45 (m, 3H), 3.40-3.35 (m, 6H), 3.25-3.15 (m, 3H), 3.06-2.86 (m, 5H), 2.20-2.00 (m, 15H), 2.00-1.70 (m, 17H), 1.60-1.45 (m, 2H), 1.35-1.25 (m, 2H), 1.20-1.10 (m, 2H).

The following conjugates were also prepared using procedures similar to the methods described above. One of skill in the art will readily appreciate and envision modifications and reagents necessary for the preparation of the following conjugates.

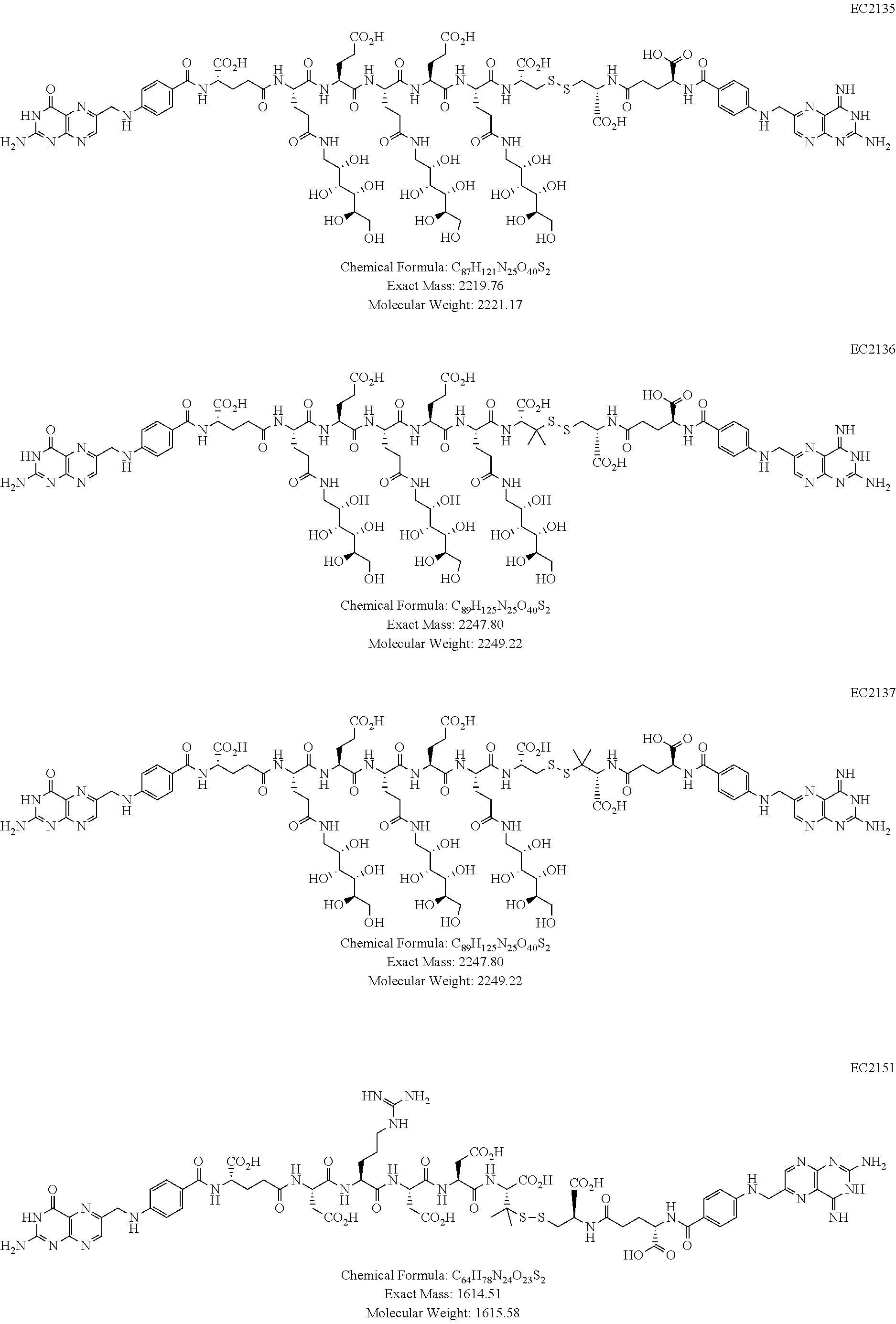

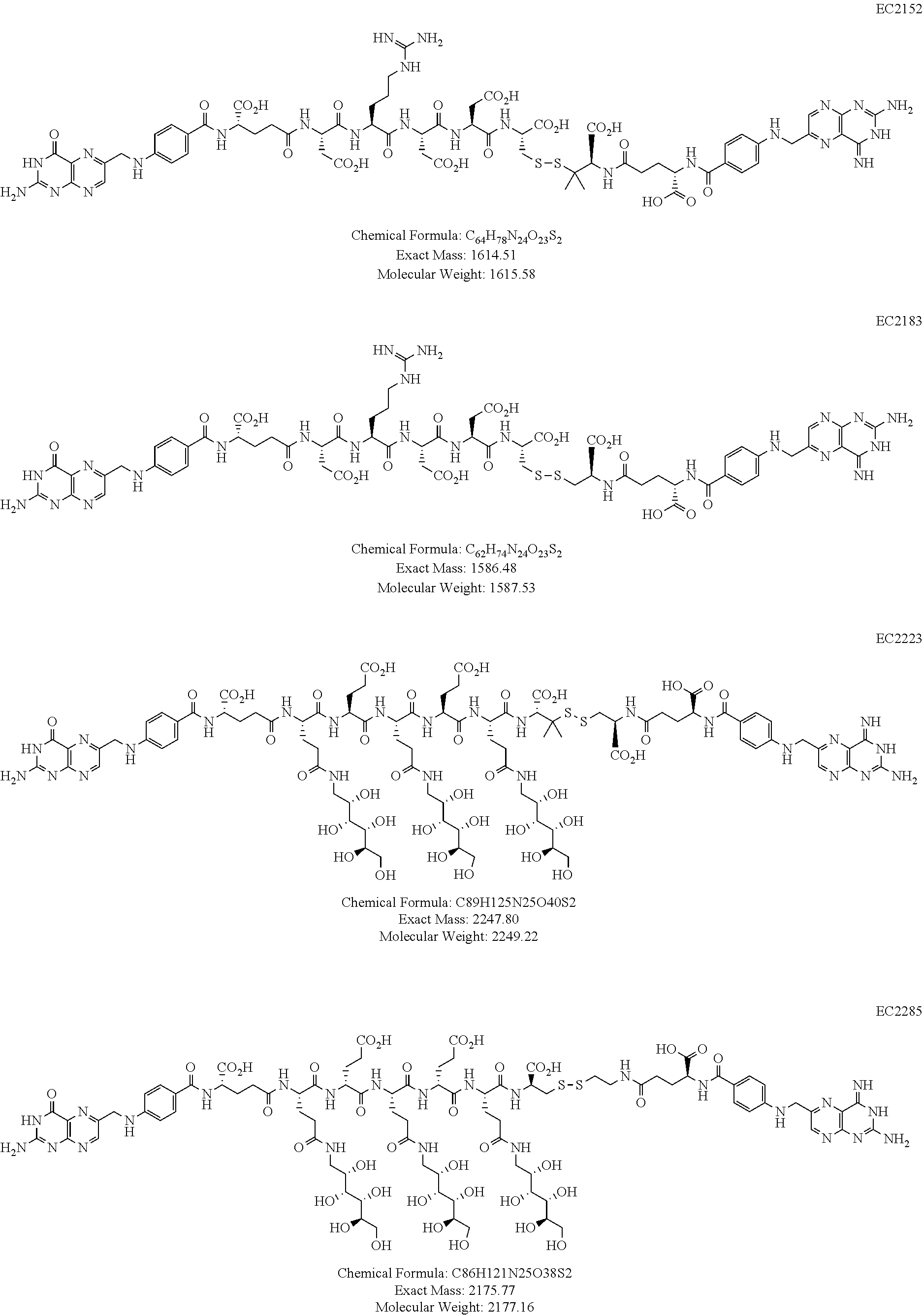

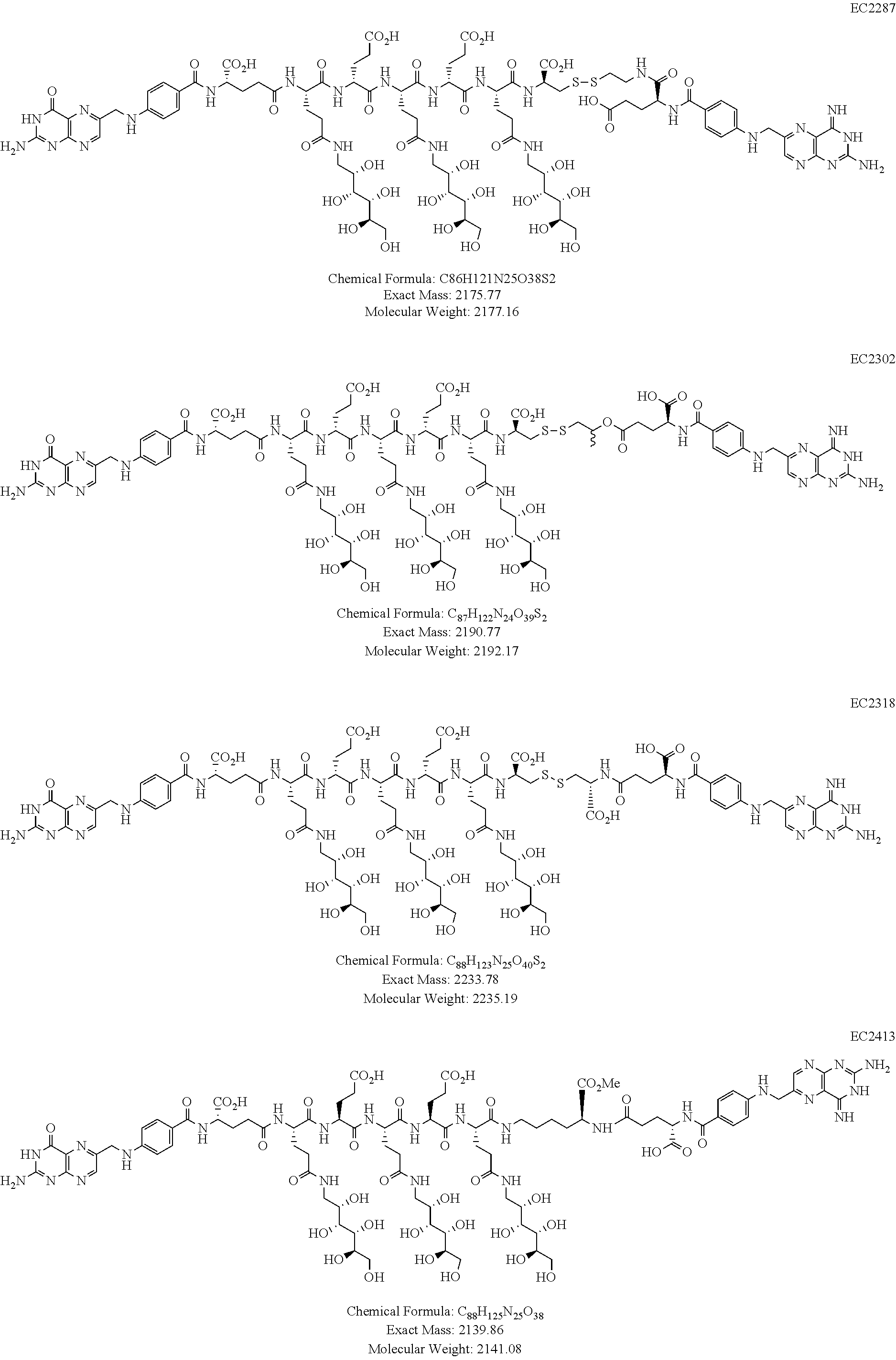

### Comparative Example 1 (EC1669)

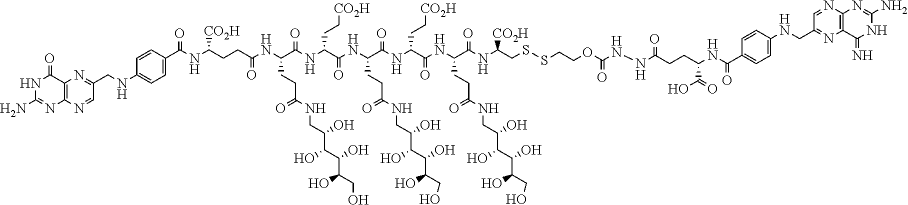 [SMILES: N=c1[nH]c(N)nc2ncc(CNc3ccc(C(=O)N[C@@H](CCC(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@H](NC(=O)c4ccc(NCc5cnc6nc(N)[nH]c(=O)c6n5)cc4)C(=O)O)C(=O)O)C(=O)O)cc3)nc12]

EC1669 can be prepared as described in WO2014/062697, and WO2012/0258905.

### Comparative Example 2 (EC2496)

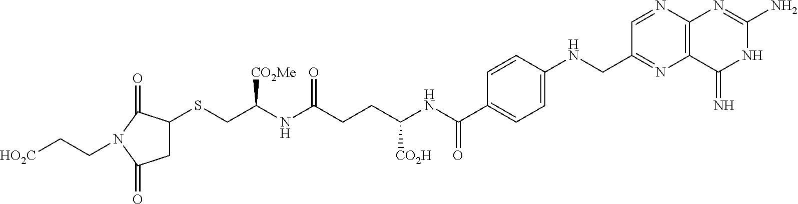 [SMILES: COC(=O)[C@H](CSC1CC(=O)N(CCC(=O)O)C1=O)NC(=O)CC[C@H](NC(=O)c1ccc(NCc2cnc3nc(N)[nH]c(=N)c3n2)cc1)C(=O)O]

### Synthesis of AMT-cys(OMe) Mercaptopyridine

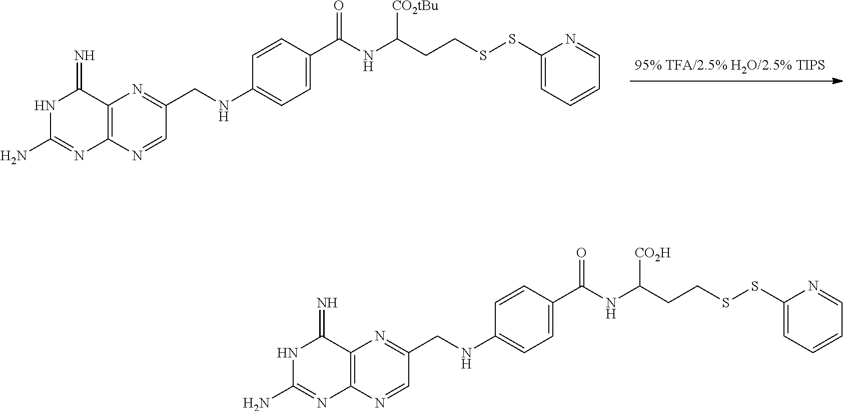 [SMILES: CC(C)(C)OC(=O)C(CCSSc1ccccn1)NC(=O)c1ccc(NCc2cnc3nc(N)[nH]c(=N)c3n2)cc1 ; N=c1[nH]c(N)nc2ncc(CNc3ccc(C(=O)NC(CCSSc4ccccn4)C(=O)O)cc3)nc12]

AMT(tBu)-cys(OMe) mercaptopyridine (60 mg, 0.083 mmol, 1 eq.) was treated with a 95%/2.5% H₂O/2.5% TIPS cleavage solution (1.6 mL). After 20 mins, UPLC (0-30% ACN/0.1% TFA pH2) showed that 90% of the starting material had been converted to the desired product. The solvent was removed under reduced pressure and the residue dried under high vacuum overnight. The crude product was collected as a red solid (63 mg) and taken into the next step without further purification.

MS (ESI): m/z 667.38amu (M+H); calc. for C₂₈H₃₁N₁₀O₆S₂: 667.18amu.

### Synthesis of EC2496

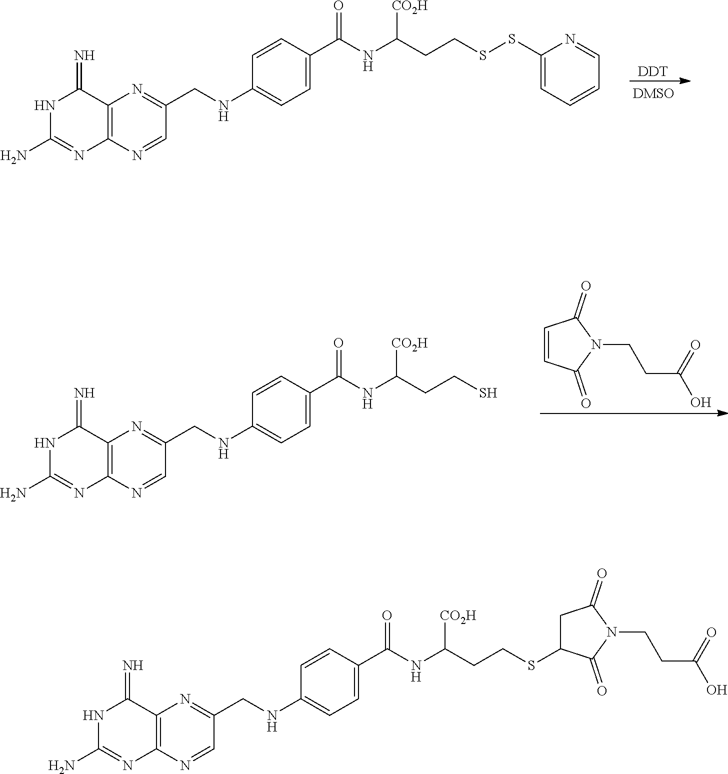

Crude AMT-cys(OMe) mercaptopyridine (33 mg, 0.050 mmol, 1 eq.) and dithiothreitol (7.6 mg, 0.050 mmol, 1 eq.) were dissolved in DMSO (0.7 mL) and argon bubbled through the solution. Reaction progress was monitored by UPLC (0-30% ACN/0.1% TFA pH2), which showed the reaction reached completion after 10 minutes. Commercially available N-Maleoyl-B-alanine (22.9 mg, 0.135 mmol, 2.7 eq.) dissolved in DMSO (0.3 mL) and triethylamine (27.6 μL, 0.198 mmol, 4 eq.) were then added to the reaction mixture. UPLC (0-30% ACN/0.1% TFA pH2) showed appearance of a single peak corresponding to the desired product. After 1 hr., the reaction mixture was purified by reverse-phase chromatography using 10-30% ACN/50 mM NH₄HCO₃ pH7 buffer as the eluent. Collection and lyophilysis of fractions containing the desired product afforded EC2496 as a yellow powder (17 mg, 47%).

MS (ESI): m/z 727.18amu (M+H); calc. for C₃₀H₃₅N₁₀O₁₀S: 727.22amu.

### Comparative Example 3 (EC1576)

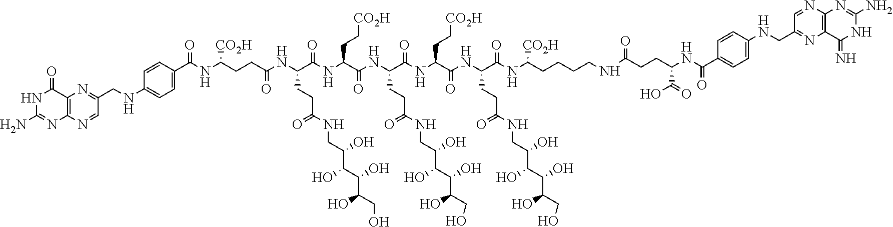 [SMILES: N=c1[nH]c(N)nc2ncc(CNc3ccc(C(=O)N[C@@H](CCC(=O)NCCCC[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CC[C@H](NC(=O)c4ccc(NCc5cnc6nc(N)[nH]c(=O)c6n5)cc4)C(=O)O)C(=O)O)C(=O)O)cc3)nc12]

### Exemplary Synthesis of EC1576

| | | | MW | Amount |
|---|---|---|---|---|
| Reagents | mmol | equivalent | (g/mol) | (g) |
| Fmoc-L-Lys(Mtt)-Wang Resin | 2.00 | | | 3.03 |
| (200-400 mesh, loading | | | | |
| 0.66 mmol/g) | | | | |
| EC0475 | 4.00 | 2 | 612.67 | 2.45 |
| Fmoc-Glu(OtBu)—OH | 4.00 | 2 | 425.47 | 1.70 |
| EC0475 | 4.00 | 2 | 612.67 | 2.45 |
| Fmoc-Glu(OtBu)—OH | 4.00 | 2 | 425.47 | 1.70 |
| EC0475 | 4.00 | 2 | 612.67 | 2.45 |
| Fmoc-Glu-OtBu | 4.00 | 2 | 425.47 | 1.70 |
| N¹⁰—TFA-Pteroic Acid | 4.00 | 2 | 408.29 | 1.63 |
| Fmoc-Glu-OtBu | 4.00 | 2 | 425.47 | 1.70 |
| HOBt-Aminopteroic Ester (61%) | 4.00 | 2 | 428.41 | 2.80 |
| DIPEA | 8.00 | 4 | | 1.03 |
| PyBOP | 4.00 | 2 | | 2.08 |

The resin was added to a peptide synthesis vessel and then the resin was swelled with DMF for 10 min. Before each amino acid coupling step, the resin was treated with 20% piperidine in DMF for Fmoc deprotection (3×20 min) and subsequently washed with 3×DMF, IPA, and DMF again. For each coupling step, the appropriate amino acid, DMF, DIPEA, and PyBOP were added to the reactor. The reaction mixture was agitated with argon bubbling for 1 hr and washed 3× with DMF, IPA, and DMF again. Continue to complete the first 7 coupling steps. To the vessel was then added 3% TFA/dichloromethane (3×10 min) and washed with 3×DMF.

Fmoc-Glu-OtBu, DMF, DIPEA, and PyBOP were added to the reactor. The reaction mixture was agitated with argon bubbling for 1 hr and washed 3× with DMF, IPA, and DMF again. The resin was treated with 20% piperidine in DMF for Fmoc deprotection (3×20 min) and subsequently washed with 3×DMF, IPA, and DMF again. HOBt-Aminopteroic Ester, DMSO, DIPEA, and PyBOP were added to the reactor. The reaction mixture was agitated with argon bubbling for 1 hr and washed 3× with DMF, IPA, and DMF again. The peptide was then cleaved from the resin by treatment of the resin with 3×20 min TFA/H₂O/TIPS (95:2.5:2.5) cleavage solution with argon bubbling. The cleavage solution was then poured into cold diethyl ether to affect precipitation of crude peptide. After isolation of the solid by centrifugation, the crude peptide was treated with aqueous sodium carbonate (pH=10) under argon bubbling for 1 hr. to cleave the TFA protecting group. The peptide was purified by preparative HPLC in 0-10% acetonitrile/50 mM ammonium bicarbonate pH7 buffer. After purification, pure EC1576 (>98% purity, 2.2 g, 52% yield) was obtained.

LC/MS conditions: 0 to 10% acetonitrile, 20 mM ammonium bicarbonate pH7.

LC/MS (ESI) [M+2H]²⁺1064.60

### Biological Examples

### 1. In-Vitro Assays

### Cell LINES

Cell lines utilized to evaluate EC2319 in in-vitro studies were as follows: KB human HeLa carcinoma contaminant expressing the human folate receptor (FR)-α, RAW264.7 mouse macrophage-derived tumor cells expressing a murine FR, THP-1-FRβ human monocytic leukemia engineered to express the human FR-β. All cells were grown in a folate-free RPMI1640 medium (Gibco BRL) (FFRPMI) containing 10% heat-inactivated fetal calf serum (HIFCS) and antibiotics, and maintained under a 5% CO₂ atmosphere using standard cell culture techniques.

### Relative Affinity Assay

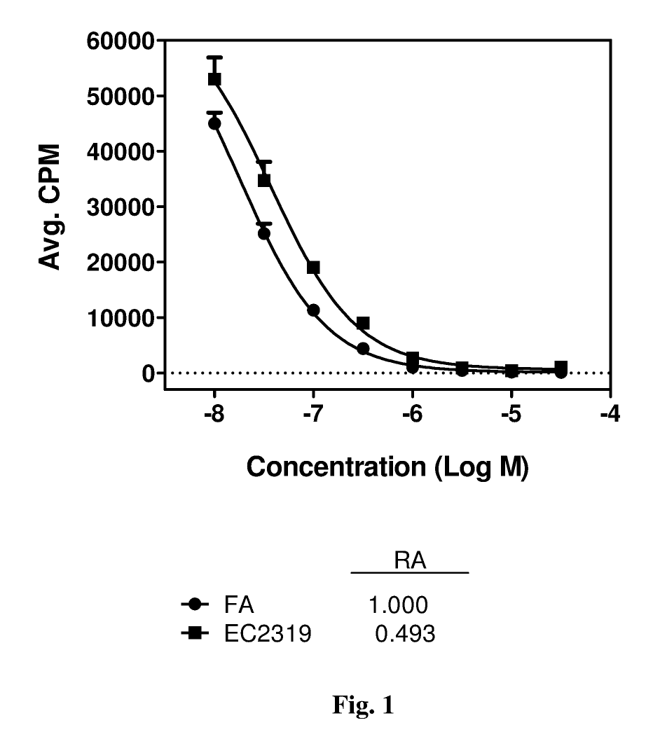

EC2319 FR-binding affinity was determined in a relative affinity assay using KB cells as the source of FR. Briefly, KB cells (1×10⁵ cells/well) were plated in 24-well plates at 18 to 24 h before use. The cells were then incubated for 1 h at 37° C. with 100 nM of ³H-folic acid (Moravek Inc.) plus a series of 3.16-fold dilutions of EC2319 or FA at 0.01-31.6 μM in triplicates. At the end of incubation, the cells were washed 3 times with a phosphate-buffered saline (PBS, pH 7.4) and lysed for 5 min at room temperature in 0.5 mL of 0.25 N NaOH. 0.45 mL of the cell lysate was taken from each well and counted in a scintillation counter. The relative affinity value was defined as the inverse molar ratio of compound or conjugate required to displace 50% of ³H-folic acid (FA) bound to FR on KB cells, and the relative affinity of FA for the FR was set to 1; that is, values <1 reflect weaker affinity than FA, and values >1 reflect stronger affinity. See result in FIG. 1.

### Cell Viability Assays

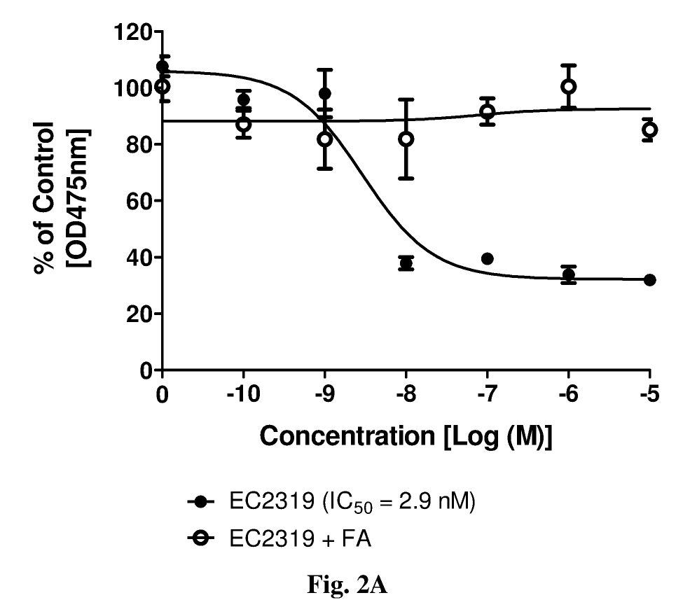

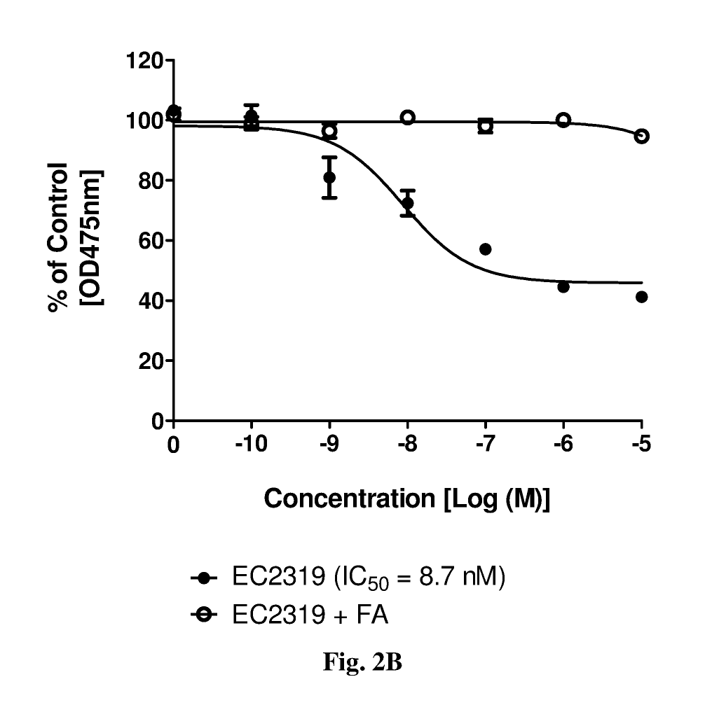

RAW264.7 cells and THP-FRβ in 96-well plates (16,000 cells/well or 75,000 cells/well, respectively) were treated with 10-fold serial dilutions of EC2319 (≤10 μM) in FFRPMI medium without and with 100-fold molar excess of FA. After a 2 h exposure, the drug-containing media were replaced and the cells were allowed to incubate further for 72 h. The cell viability was assessed by adding XTT (2,3-bis(2-methoxy-4-nitro-5-sulfo-phenyl)-2H-tetrazolium-5-carboxanilide) to the culture medium for 3 h following the manufacturer's instructions. All results were expressed as % absorbance (minus background) relative to the untreated control cells. See results in FIG. 2A and FIG. 2B.

EC2319 was evaluated for its anti-proliferative activity against mouse RAW264.7 macrophage cells and human THP-1-FRβ cells. Both cell lines were exposed for 2 h to 10-fold serial dilutions of EC2319 (0.1 nM-10 μM) without or with 100-fold excess FA and followed by a 72 h chase in drug-free medium. As determined by the XTT assay, EC2319 showed a dose-dependent inhibition of cell proliferation with relative IC₅₀ values of ˜2.9 nM and ˜8.7 nM on RAW264.7 (FIG. 2A) and THP-1-FRβ (FIG. 2B) cells respectively. Importantly, the observed anti-proliferative effect was 100% competable in the presence of excess FA, indicating a FR-specific mode of action. Furthermore, EC2319 appeared to have a cytostatic effect on RAW264.7 and THP-1-FRβ cells at concentrations ≥10 nM (FIG. 2A) and ≥100 nM (FIG. 2B), respectively. Taking together, these data demonstrated that EC2319 halted the proliferation of RAW264.7 and THP-1-FRβ cells in a FR-dependent manner.

### 2. In-Vivo Studies

### Rats

Female Lewis rats (175-200 g) were purchased from Harlan Sprague Dawley (Indianapolis, Ind.) and allowed to acclimate for 1 week. Generally, rats were fed a folate-deficient diet (Harlan Teklad) for 9-10 days prior to arthritis induction.

### Induction and Assessment of Rat Adjuvant Arthritis

Rat adjuvant arthritis (AIA) was induced by intradermal inoculation (at the base of tail) of 0.45-0.46 mg of heat-killed Mycobacteria butyricum (BD Diagnostic Systems, Sparks, Md.) in 100 μL light mineral oil (Sigma). The onset of arthritis usually occurred 9-11 days after induction with distinctive but mild redness and/or swelling in small areas of the foot. During the course of disease development, animals were weighed at least 3 times a week. Paw edema (degree of arthritis) were scored 3 times a week as follows: 0=no edema or arthritis; 1=swelling in one type of joint; 2=swelling in two types of joint; 3=swelling in three types of joint; 4=swelling of the entire paw. A total score for each rat is calculated by adding up the scores for each of the four paws, giving a maximum of 16 per animal.

### Treatment of AIA Rats

On the first day of treatment, rats with desired arthritis scores were distributed evenly across the control and treatment groups (n=5). Generally, 2 rats from the same colony were not induced arthritis and used as healthy controls. All compounds and conjugates were administered subcutaneously (s.c.) starting 9 days after arthritis induction with biweekly (BIW, Mondays and Thursdays) or once-a-week (QW, Mondays) dosing for two consecutive weeks. At the completion of each study (˜4 days after the last treatment), rats were euthanized by CO₂ asphyxiation and processed for paw weight (cut at the hairline) and spleen weight. Using 500 nomol/kg (QW) and/or 1000 nmol/kg (BIW) dosing regimens, EC2319 was compared against a series of small molecule folate-aminopterin conjugates with alternative linker chemistries (EC1669, EC2285, EC2318, and EC2413). To determine FR-specific anti-inflammatory mechanism of EC2319 in vivo, a therapeutically irrelevant folate-containing competitor (EC0923, MW 672) was used in 500-fold molar excess to block the activities of EC2319 at 500 nmol/kg (BIW).

### Study 1:

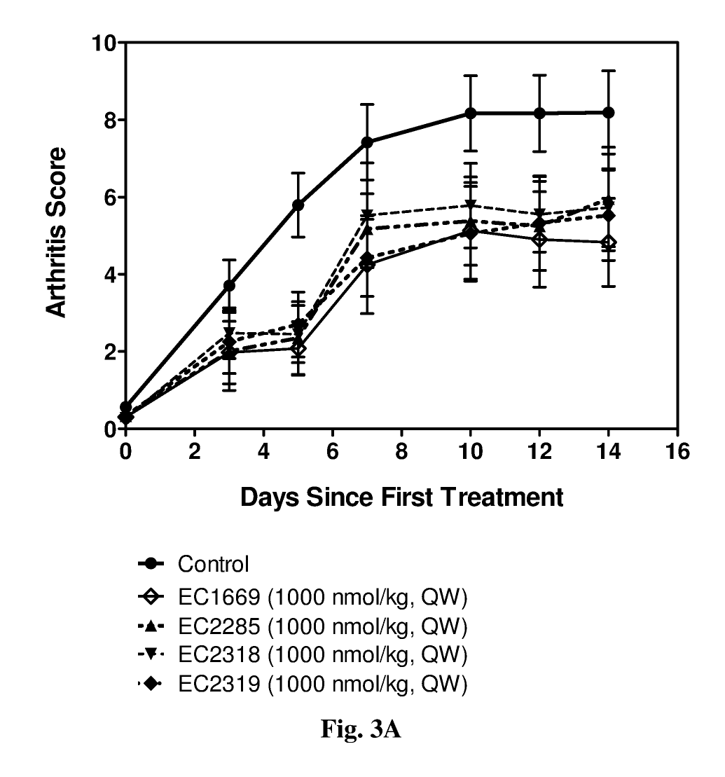

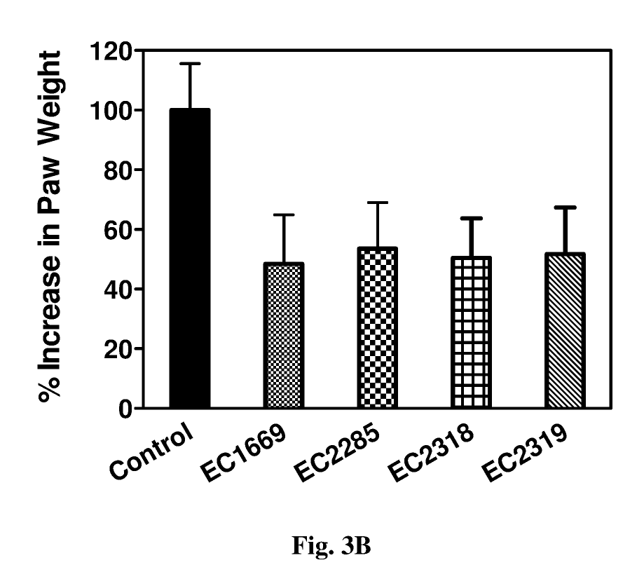

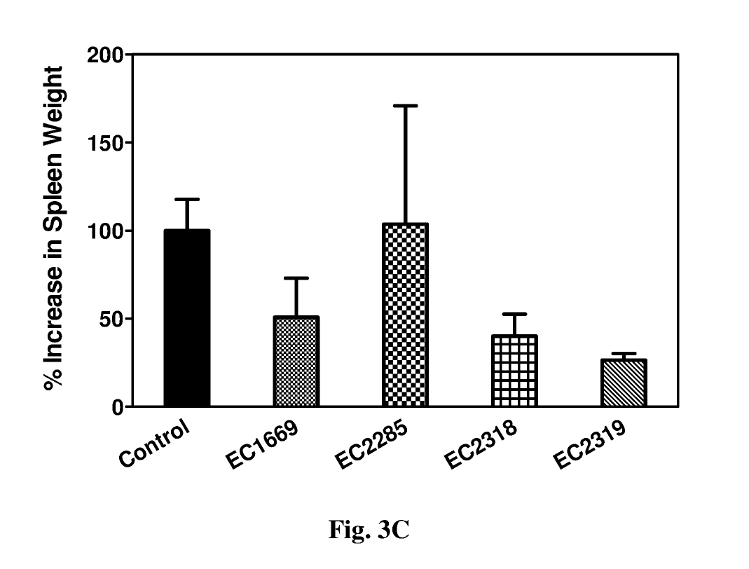

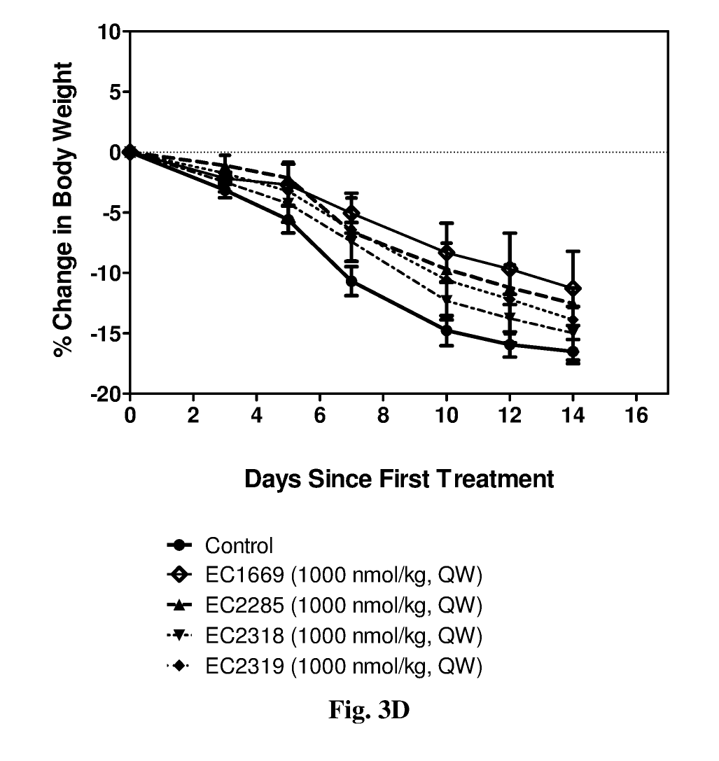

The rat AIA model resembles many characteristics of human rheumatoid arthritis and it is very aggressive. In this study, rats with developing AIA (9 days after induction) were distributed according to arthritis scores into five groups: (1) the untreated AIA control (n=6), (2) EC1669 (n=5), (3) EC2285 (n=5), (4) EC2318 (n=5), and (5) EC2319 (n=5). The animals in the AIA control group were left untreated. The animals in designated groups were dosed with EC1669, EC2285, EC2318, or EC2319 at equal molar doses (1000 nmol/kg, QW) for two consecutive weeks. EC2319 was found as effective as EC1669, EC2285, and EC2318 in alleviating AIA symptoms, such as increased arthritis score (FIG. 3A), paw weight (FIG. 3B), and spleen weight (FIG. 3C). Notably, one rat had an enlarged spleen in the EC2285 group likely due to opportunistic infection (FIG. 3C). In addition, only small improvements in body weight loss were seen in all treatment animals due to the aggressiveness of this model and the infrequent QW dosing (FIG. 3D).

### Study 2:

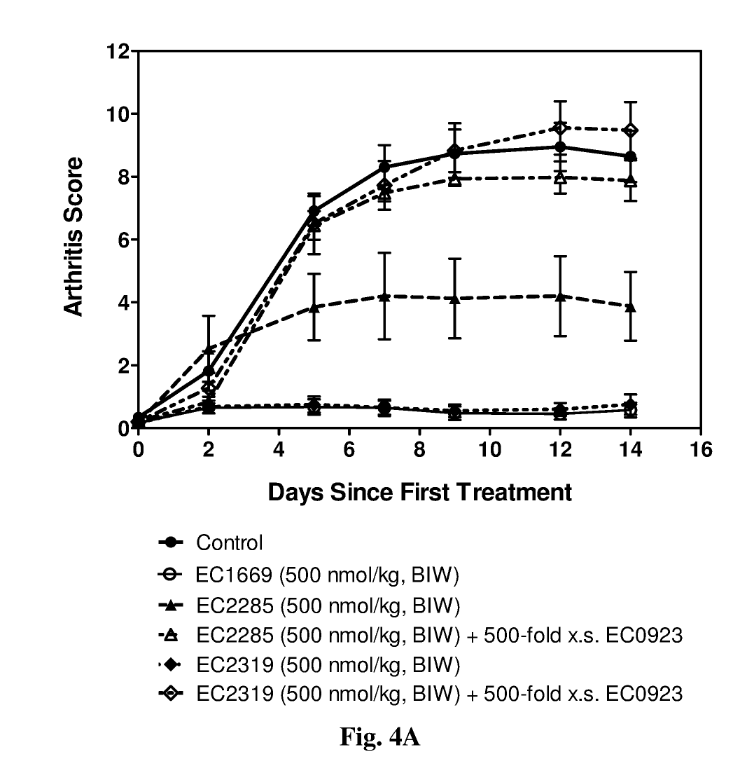

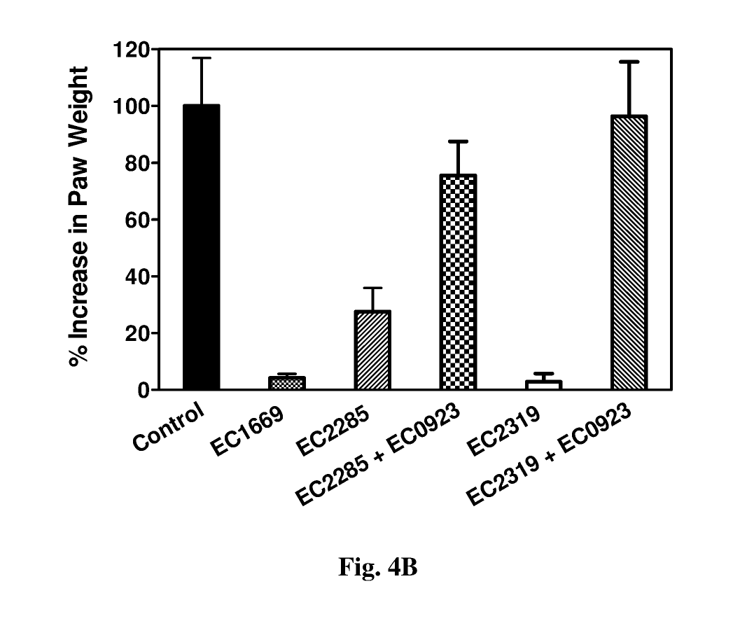

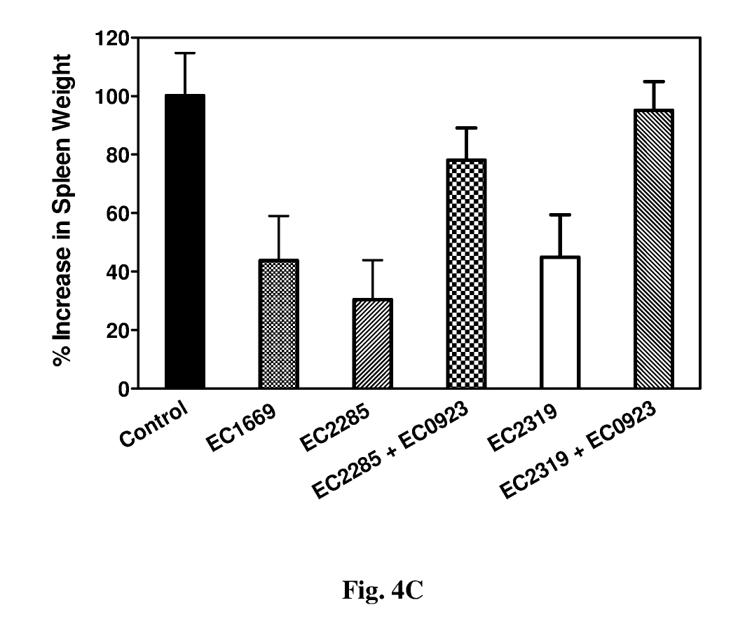

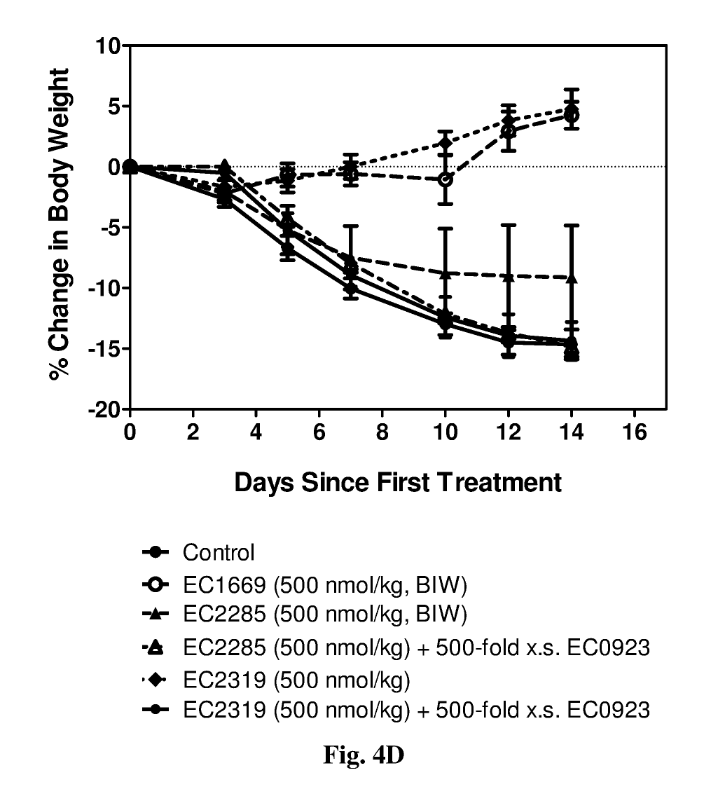

In this study, EC2319 was compared against EC1669 and EC2285 at 500 nmol/kg (BIW) for two consecutive weeks. In addition, EC0923, a benign folate-containing competitor was used in conjunction to EC2319 and EC2285 to block their FR binding capabilities in-vivo. EC0923 (pteroyl-γGlu-D-Asp-D-Asp) is a high affinity water-soluble FA-peptide conjugate that is used for in vivo competition studies rather than FA because high doses of the latter can cause renal damage due to precipitation in the kidneys. Thus, AIA rats were distributed according to arthritis scores into six groups: (1) the untreated AIA control (n=7), (2) EC1669 (n=5), (3) EC2285 (n=5), (4) EC2285 plus EC0923 (n=5), (5) EC2319 (n=5), and (6) EC2319 plus EC0923 (n=5). Only animals in the EC2285 plus EC0923 group and the EC2319 plus EC0923 group received a concurrent dose of EC0923 at 500-fold molar excess (250 μmol/kg). EC2319 was found equally efficacious as EC1669 but significantly more active than EC2285 in reducing arthritis scores (FIG. 4A) and paw weights (FIG. 4B). Importantly, the anti-arthritic activities of EC2319 and EC2285 were fully blocked by EC0923 based on arthritis score (FIG. 4A), paw weight (FIG. 4B), spleen weight (FIG. 4C), and body weight (FIG. 4D). With the BIW dosing regimen, the animals treated with EC2319 and EC1669 had minimal residual diseases and therefore maintained a healthier body weight than EC2285 (FIG. 4D).

### Study 3:

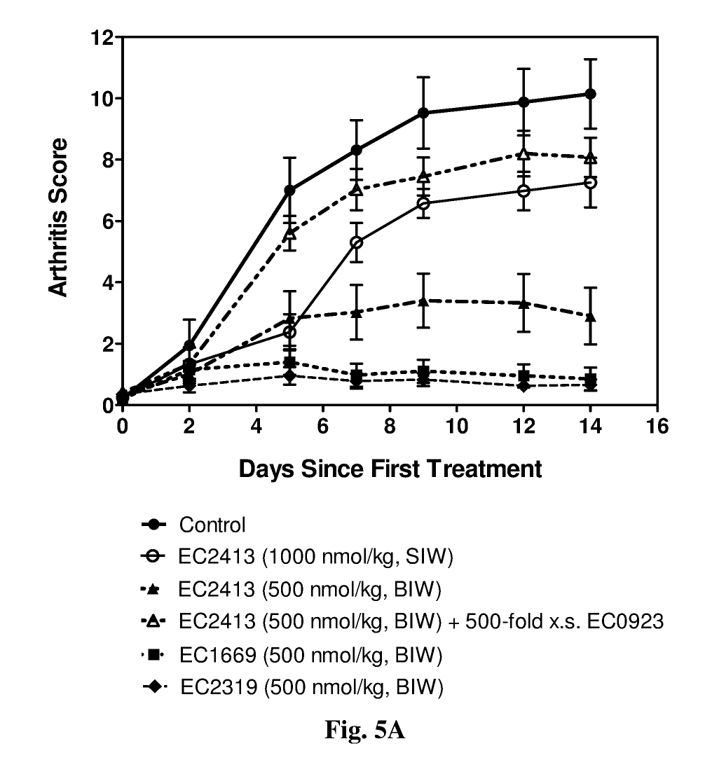

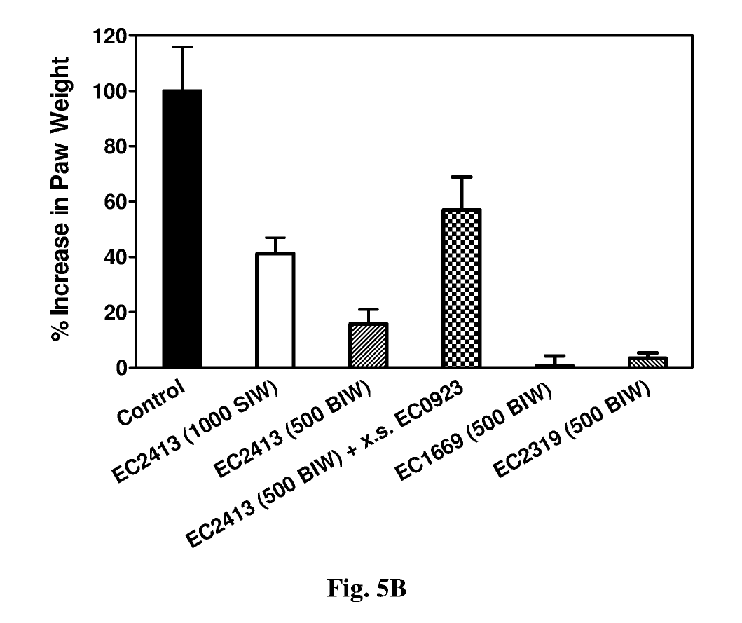

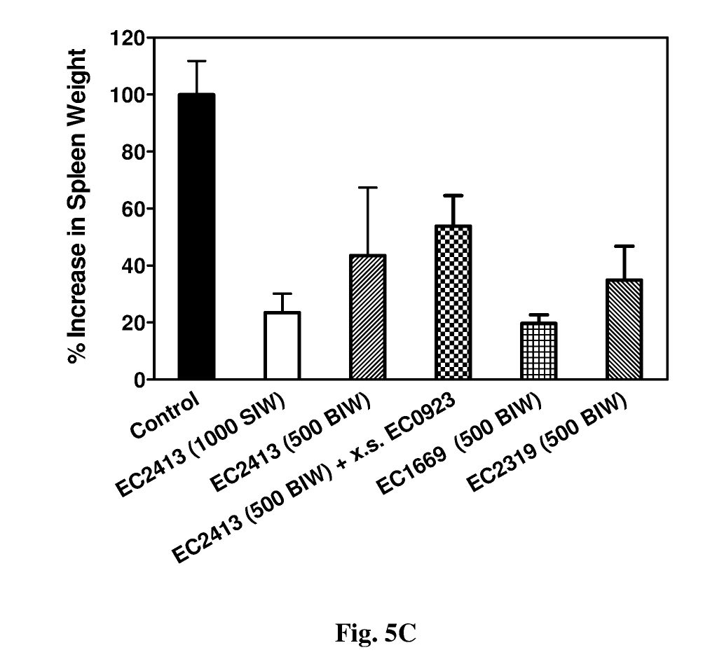

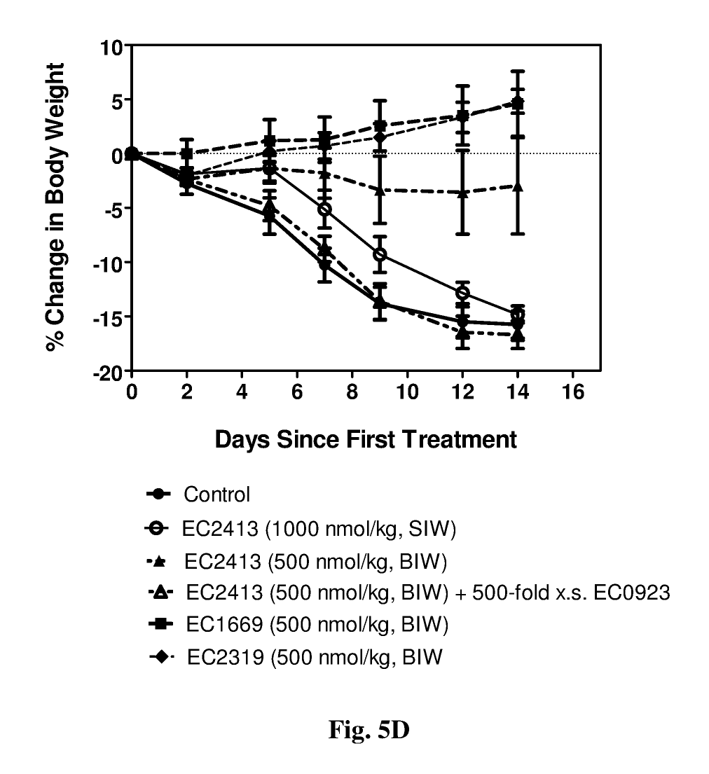

In a subsequent study, EC2319 (500 nmol/kg, BIW) was compared against EC1669 (500 nmol/kg, BIW), EC2413 (500 nmol/kg, BIW), and EC2413 (1000 nmol/kg, SIW) for two consecutive weeks. Here, AIA rats were distributed according to arthritis scores into six groups: (1) the untreated AIA control (n=6), (2) EC2413 at 1000 nmol/kg (QW, n=5), (3) EC2413 at 500 nmol/kg (BIW, n=5), (4) EC2285 at 500 nmol/kg plus a 500-fold excess of EC0923 (BIW, n=5), (5) EC1669 at 500 nmol/kg (BIW, n=5), and (6) EC2319 at 500 nmol/kg (BIW, n=5). EC0923 was used to block FR-specific activity of EC2413 at 500 nmol/kg (BIW) in-vivo. When dosed at 500 nmol/kg (BIW), EC2319 was as efficacious as EC1669 in decreasing arthritis score (FIG. 5A), paw weight (FIG. 5B), and spleen weight (FIG. 5C). Under the same conditions, EC2413 was found inferior to both EC2319 and EC1669, but all three conjugates had significant anti-arthritic activity and the animals maintained a good body weight (FIG. 5D). In all parameters assessed, infrequent EC2413 dosing at 1000 nmol/kg (QW) was less effective than EC2413 dosed at 500 nmol/kg (BIW). Thus, more frequent dosing is needed in controlling diseases progression this aggressive animal model.

### 3. Pharmacokinetics Studies

a. Pharmacokinetics in Rats:

EC1669 and EC2319 were each administered subcutaneously to female Lewis rats with rounded tip jugular vein catheters (Harlan Laboratories, Indianapolis, Ind.) at a dose of 500 nmol/kg (1.118 mg/kg for EC1669 and 1.132 mg/kg for EC2319). Each rat was used for collection of blood samples for a maximum of 4 time points. Blood samples were collected at 1, 10, and 30 minutes, 1, 2, 3, 4, 8, and 12 hours after dosing for EC1669 and at 1, 10, and 30 minutes, 1, 2, 3, 4, 8, 12, and 19 hours after dosing for EC2319, into tubes containing 1.7 mg/mL K3EDTA, 0.425 mg/mL N-Maleoyl-β-alanine, 1 mg/mL mannitol, and 0.00375% acetic acid. The samples were centrifuged for 3 minutes at 2000×g (Eppendorf 5417R centrifuge) to obtain plasma. The plasma samples were stored at −80° C. until LC-MS/MS analysis.

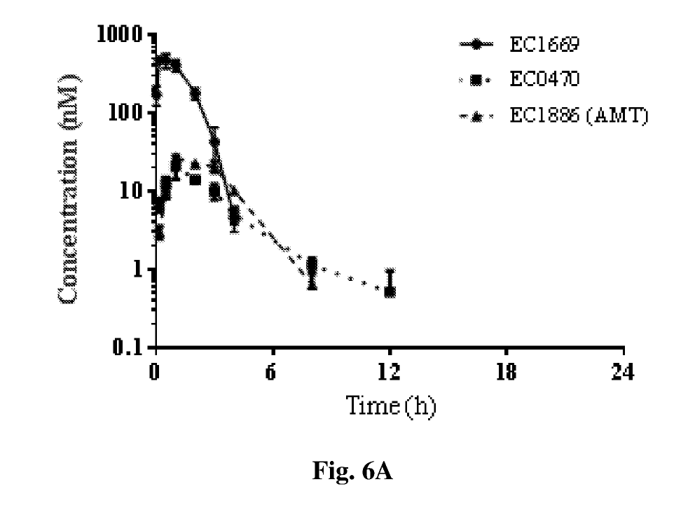

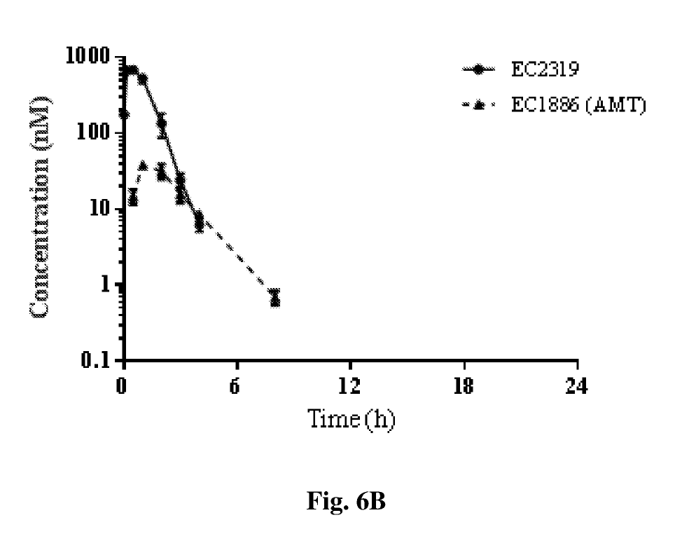

Results: Pharmacokinetic parameters for plasma EC1669 and EC2319, on subcutaneous dosing at 500 nmol/kg in rats, are shown in Table 1 and plotted in FIG. 6A and FIG. 6B respectively. Plasma concentration-time profiles for both conjugates and for released aminopterin (EC1886) appeared to be identical (FIG. 6A and FIG. 6B). For both conjugates, plasma levels of the conjugates were quantifiable up to 4 hours post dosing. Time of maximal observed plasma concentration (Tₘₐₓ) was 0.5 h for both the conjugates. Terminal half-life estimates (t_{1/2}) were similar for both EC1669 (0.464 h) and EC2319 (0.463 h). Peak plasma concentration (Cₘₐₓ) and AUC values for EC2319 appeared slightly higher than those for EC1669. Similarly, PK parameters for aminopterin released from both conjugates appeared similar.

TABLE 1
Pharmacokinetic Parameters for EC1669 and EC2319 Dosed Subcutaneously in Rats

| Compound | | Dose | | t_{1/2} | Tₘₐₓ | Cₘₐₓ | AUCₗₐₛₜ | AUC_{inf} |
|---|---|---|---|---|---|---|---|---|
| Dosed | Dosing | (nmol/kg) | Compound | (h) | (h) | (nM) | (nM*h) | (nM*h) |
| EC1669 | SC | 500 | EC1669 | 0.464 | 0.5 | 472 | 857 | 860 |
| | | | EC0470 | 1.64 | 1 | 21.2 | 64.7 | 65.9 |
| | | | EC1886 | 0.997 | 1 | 27.1 | 94.7 | 95.6 |
| EC2319 | SC | 500 | EC2319 | 0.463 | 0.5 | 682 | 1017 | 1021 |
| | | | EC1886 | 1.11 | 1 | 38.2 | 106 | 107 |

b. Pharmacokinetics in Dogs:

The pharmacokinetics of EC1669 in dogs was determined as part of a twenty eight day subcutaneous range finding study of EC1669 in beagle dogs conducted at BASi (Mt. Vernon, Ind.; Study Number 0157-13117). EC1669 was administered subcutaneously at various doses including a dose of 2.4 mg/kg, data for which is shown in Table X. Blood samples were collected from the peripheral vein at predose, 15, 30, and 45 minutes, 1, 2, 3, 4, 8, and 24 hours after dosing in tubes containing K₃EDTA fortified with N-Maleoyl-β-alanine, mannitol, and acetic acid. The samples were centrifuged under refrigeration for at least 10 minutes at 3000 rpm and the plasma generated stored at −20° C. until LC-MS/MS analysis.

EC2319 was administered intravenously and subcutaneously at doses of 1 mg/kg and 2.43 mg/kg respectively to male beagles as part of study 0157-14059 conducted at BASi (Mt. Vernon, Ind.), Blood samples were collected from the peripheral vein at predose, 2, 5, 15, and 30 minutes, 1, 2, 4, 8, and 12 hours after dosing for the intravenous dose and at predose, 15, 30, and 45 minutes, 1, 2, 3, 4, 8, and 24 hours after dosing for the subcutaneous dose, in tubes containing K₃EDTA fortified with N-Maleoyl-β-alanine, mannitol, and acetic acid. The samples were centrifuged under refrigeration for at least 10 minutes at 3000 rpm and the plasma generated stored at −20° C. until LC-MS/MS analysis.

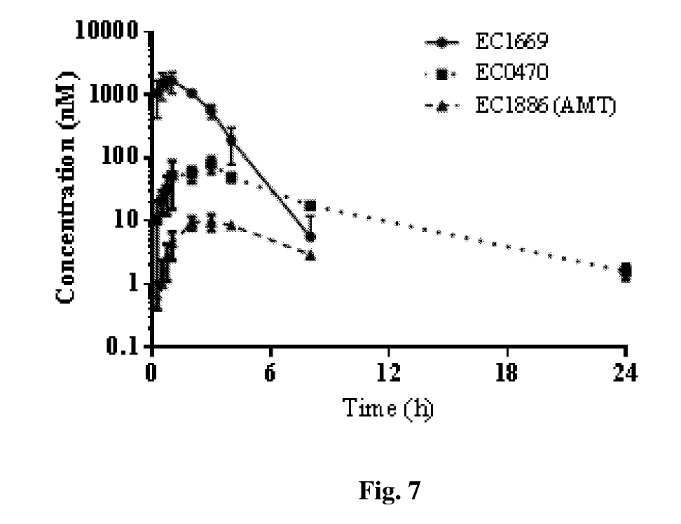

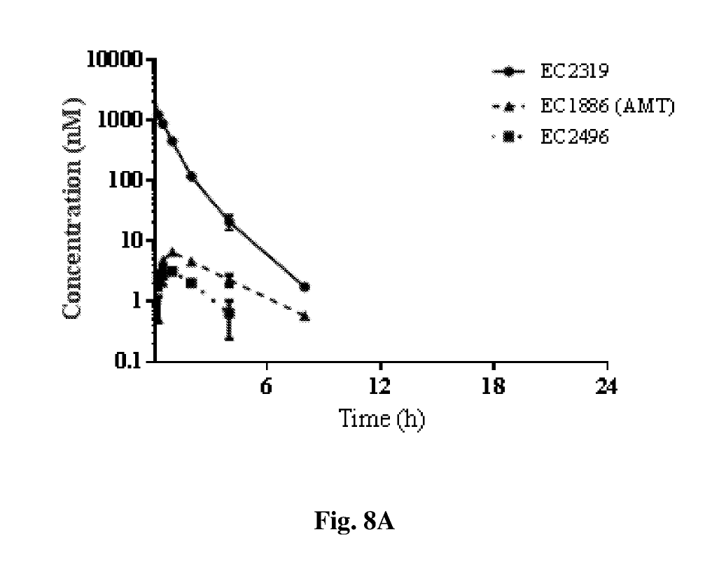

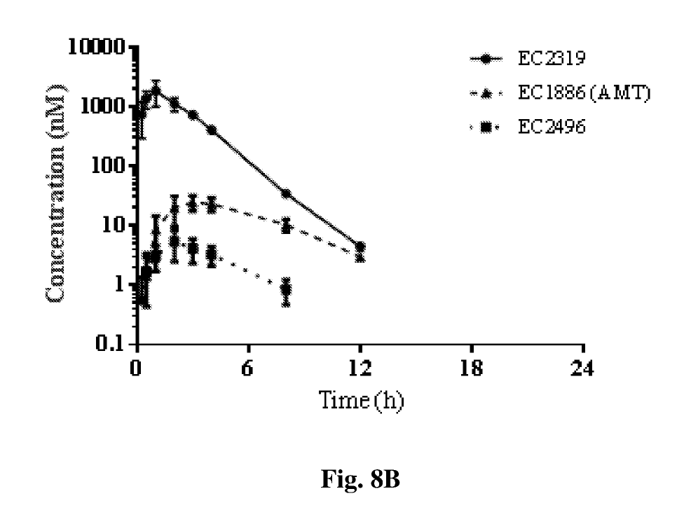

Results: Pharmacokinetic parameters for plasma EC1669 on subcutaneous dosing, and for EC2319, on subcutaneous and intravenous dosing in dogs, are shown in Table 2 and plotted in FIGS. 7 and 8A and 8B respectively. Time of maximal observed plasma concentration (Tₘₐₓ) on subcutaneous dosing was 1.19 h for EC1669 and 1.00 h for EC2319. Terminal half-life estimates (t_{1/2}) were similar for both EC1669 (0.994 h) and EC2319 (1.21 h). Peak plasma concentration (Cₘₐₓ) and AUC values for EC2319 appeared slightly higher than those for EC1669. However, the Cₘₐₓ value for aminopterin released was nearly 2.6-fold higher and AUCₗₐₛₜ nearly 3.8-fold higher from EC2319 than from EC1669.

TABLE 2
Pharmacokinetic Parameters for EC1669 Dosed Subcutaneously and for
EC2319 Dosed Intravenously and Subcutaneously in Beagle Dogs

| | | | Dose | Dose | | t_{1/2} | Tₘₐₓ | Cₘₐₓ | AUCₗₐₛₜ | Vz | Cl | |
|---|---|---|---|---|---|---|---|---|---|---|---|---|
| Ex | Dosing | N | (mg/kg) | (nmol/kg) | Cpd | (h) | (h) | (nM) | (nM*h) | (L/kg) | (L/h/kg) | % F |
| EC1669 | SC | 4 | 2.4 | 1073 | EC1669 | 0.994 ± | 1.19 ± | 1389 ± | 4072 ± | | | |
| | | | | | | 0.427 | 0.554 | 496 | 350 | | | |
| | | | | | EC0470 | 4.38 ± | 3.25 ± | 66.5 ± | 510 ± | | | |
| | | | | | | 0.577 | 0.500 | 22.3 | 94.7 | | | |
| | | | | | EC1886 | ND | 3.50 ± | 9.41 ± | 41.7 ± | | | |
| | | | | | | | 0.577 | 1.76 | 15.6 | | | |
| EC2319 | SC | 2 | 2.43 | 1074 | EC2319 | 1.21 ± | 1.00 ± | 1845 ± | 5065 ± | | | 133% |
| | | | | | | 0.020 | 0 | 847 | 1148 | | | |
| | | | | | EC2496 | 2.14 ± | 2.00 ± | 5.38 ± | 22.6 ± | | | |
| | | | | | | 0.047 | 0 | 2.90 | 10.1 | | | |
| | | | | | EC1886 | 2.74 ± | 3.00 ± | 24.5 ± | 157 ± | | | |
| | | | | | | 0.244 | 0 | 7.06 | 48.1 | | | |
| EC2319 | IV | 2 | 1 | 442 | EC2319 | 1.01 ± | 0.03 ± | 2958 ± | 1565 ± | 0.410± | 0.282 ± | |
| | | | | | | 0.006 | 0 | 72.0 | 27.0 | 0.009 | 0.005 | |
| | | | | | EC2496 | 1.28 ± | 0.75 ± | 3.43 ± | 7.80 ± | | | |
| | | | | | | 0.44 | 0.35 | 0.055 | 0.281 | | | |
| | | | | | EC1886 | 2.00 ± | 1.00 ± | 6.63 ± | 21.8 ± | | | |
| | | | | | | 0.023 | 0 | 0.595 | 2.59 | | | |

c. Preparation of Whole Cell Lysates from Folate Receptor Positive Thioglycollate Induced Inflammatory Rat Peritoneal Macrophages:

Female Lewis rats approximately 200 grams in size (Harlan Laboratories, Indianapolis, Ind.) were injected intraperitoneally at 20 mL/kg of body weight with sterile 7.5% thioglycollate solution (BD Biosciences) supplemented with 12.5% BSA aged for more than 6 months in the presence of 0.5 M D-glucose at 37° C. in the dark according to the procedure of Li et al. (Journal of Immunological Methods (1997) 201:183-188). Three days later these rats were humanely euthanized with CO₂ asphyxiation and total peritoneal cells isolated by intraperitoneal lavage with 50 mL of sterile phosphate buffered saline (PBS) containing 0.5 mM EDTA. Red blood cells were lysed with a 5 minute incubation with 1×RBC lysis buffer (BioLegend, San Diego, Calif.). Cells were washed with PBS then plated in a T-175 tissue culture treated plate at a density of 12.5 million total live cells (as determined by trypan blue exclusion) in 10% fetal calf serum containing folic acid deficient RPMI 1640 media (Mediatech, Manassas, Va.). Cells were incubated for 2 hrs in 5% CO₂. After two hours, floating cells/debris was removed and the adherent cells were washed once with drug free media. Normal growth media (10% FCS RPMI1640) was added to each plate of cells and then incubated for 1, 2 and 3 days in the tissue culture incubator. The adherent cells were harvested using an 8 minute trypsin digest to loosen the cells and then gently scraped off the plate. Importantly the vast majority of cells were intact as seen by trypan blue exclusion of the cells. Live cells were counted and then washed once with cold PBS. Cells were then resuspended in 200 μL of cold PBS which contained no protease inhibitors and then sonicated with 3 rounds of 5 second pulses at 20% amplification with a Branson model 450 digital sonifier. After sonication to lyse cells, the lysates were resuspended in PBS to make a concentration of cell lysates equivalent to 11.1 million cells/mL of PBS.

d. Preparation of Whole Cell Lysates from FR+ Peritoneal Macrophages Derived from Rats with Adjuvant Induced Arthritis:

Prior to immunization with adjuvant, female Lewis rats were fed a folate-deficient diet (Harlan Teklad, Indianapolis, Ind.) for approximately 10 days to reduce serum folate competition from high-folate-containing regular rodent chow. The rats were then inoculated intradermally (at the base of tail) with 0.5 mg heat-killed M. butyricum (BD Diagnostic Systems, Franklin Lakes, N.J.) in 100 μL light mineral oil (Sigma-Aldrich, St Louis, Mo., USA). The rats were then allowed to develop arthritis scores between 3 and 4 as described in Lu et al. (Arthritis Research & Therapy 2011, 13:R56). After rats developed severe joint inflammation, AIA rat peritoneal cells were isolated, plated, and whole cell lysates were prepared as described above with the exception that the lysates were resuspended in 0.1 M sodium acetate buffer, pH 4.5, to make a concentration of cell lysates equivalent to 10 million cells/mL of 0.1 M sodium acetate buffer, pH 4.5.

e. Preparation of Whole Cell Lysates from Folate Receptor Positive RAW264.7 Cells:

Mouse macrophage-like RAW264.7 cells which have previously been shown to express high levels of folate receptor were grown in 10% fetal calf serum containing folic acid deficient RPMI 1640 media (Mediatech, Manassas, Va.), harvested and cell lysates were prepared as described above with the exception that the lysates were resuspended in 0.1 M sodium acetate buffer, pH 4.5, to make a concentration of cell lysates equivalent to 10 million cells/mL of 0.1 M sodium acetate buffer, pH 4.5.

f. Preparation of Whole Cell Lysates from Folate Receptor Positive THP1-FRβ Cells

Human monocyte-like THP1 cells which were previously stably transfected with human folate receptor β were grown in 10% fetal calf serum containing folic acid deficient RPMI 1640 media (Mediatech, Manassas, Va.), harvested and cell lysates were prepared as described above with the exception that the lysates were resuspended in 0.1 M sodium acetate buffer, pH 4.5, to make a concentration of cell lysates equivalent to 10 million cells/mL of 0.1 M sodium acetate buffer, pH 4.5.

g. Incubation of EC2319 and EC1669 with Rat, Dog, and Human Hepatic Cytosol:

EC2319 and EC1669 were incubated in 5% rat, dog, and human hepatic cytosol at different pH's to look at release of aminopterin from these conjugates. Liver cytosols from male Sprague-Dawley rats (Lot No. 1110428), male beagle dogs (Lot no. 1310024), and male humans (Lot No. 0710493), all containing 10 mg/mL protein, were obtained from Xenotech LLC (Lenexa, Kans.). These were diluted 20× in either 0.5 M sodium acetate buffer, pH 4.5, 0.5 M sodium acetate buffer, 6.0, or 0.5 M potassium phosphate buffer, pH 7.4 in a final volume of 500 μL. Reactions were initiated by the addition of 1 μL of either EC2319 or EC1669 and the reaction mixtures incubated at 37° C. for 1 hour. At the end of the incubation, a 100 μL aliquot was withdrawn into a cluster tube and the reaction was terminated by the addition of 5 μL of stabilizer solution (containing 8.5 mg/ml N-Maleoyl-β-alanine, 20 mg/mL mannitol and 0.075% acetic acid) and 100 μL of acetonitrile containing d₅-aminopterin. The tubes were vortexed and then centrifuged at 4000 rpm for 10 minutes (Eppendorf centrifuge 5810R). 150 μL of the supernatant was transferred to 96-well plates and the acetonitrile evaporated off under nitrogen at 35° C. for 5 minutes. The extract was diluted with 50 μL of mobile phase A. The plate was vortexed on a VX-2500 multi-tube vortexer (VWR International, Radnor, Pa.) and the extract analyzed by LC-MS/MS.

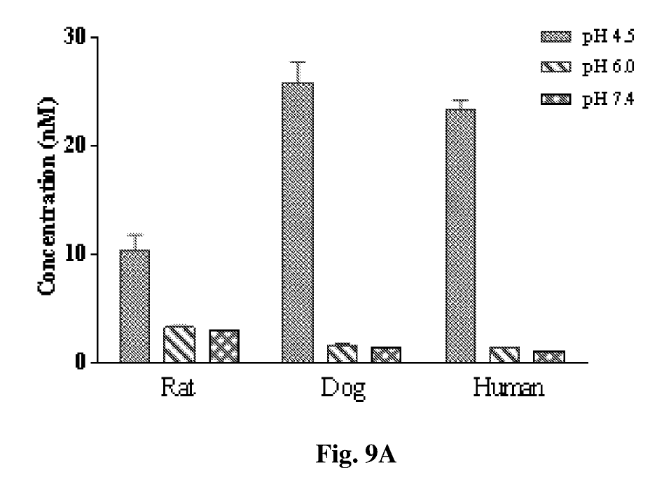

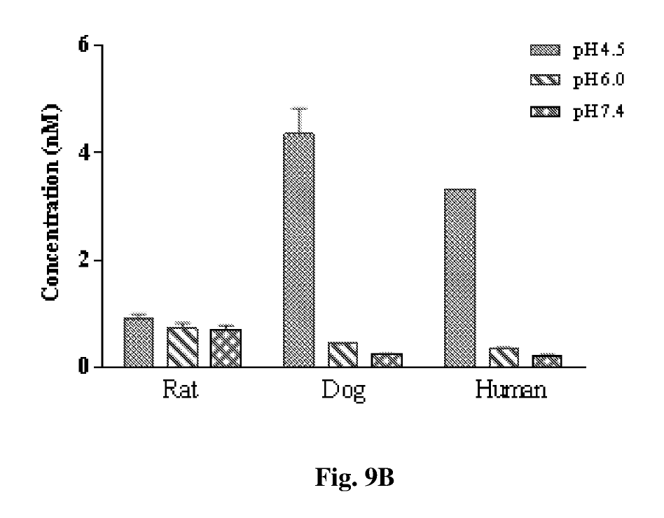

Results: The release of aminopterin from EC2319 and EC1669 was evaluated by incubation of the conjugates in 5% rat, dog, and human liver cytosol at pH 4.5, 6.0, and 7.4. As shown in FIGS. 9A and 9B, the overall release profiles of aminopterin from both conjugates were similar, though the magnitude of release from EC2319 appeared lower than that from EC1669. There appeared to be species differences in the release of aminopterin from the conjugates. In dog and human liver cytosol, release of aminopterin from the conjugates was highest at pH 4.5 and much less at pH 6.0 or 7.4. On the other hand, there was a broad pH specificity of aminopterin release from both conjugates in rat liver cytosol.
h. Incubation of EC2319 and EC1669 with Gamma-Glutamyl Hydrolase:

An incubation mixture of 100 μL contained 0.1 M sodium acetate, pH 4.5, 20 mM dithiothreitol (DTT), 1 μM EC2319 or EC1669 and 0.09 ng recombinant gamma-glutamyl hydrolase (Abnova, Taipei, Taiwan, Lot E8291) was prepared. After incubation for 2 hrs at 37° C., the reaction was terminated by the addition of 5 μL of stabilizer solution and 100 μL of acetonitrile containing d₅-aminopterin (Endocyte, Inc.). The rest of the workup is as described above.

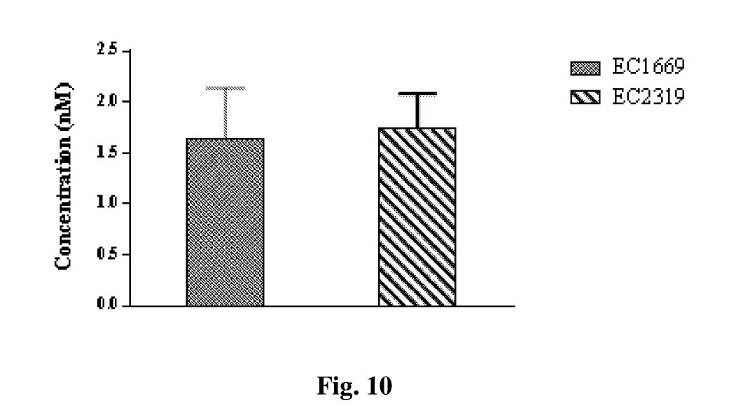

Results: EC1669 and EC2319 were incubated with recombinant human gamma-glutamyl hydrolase (rGGH) to test the hypothesis that it could be involved in the release of aminopterin from the conjugates. As can be seen from FIG. 10, similar amounts of aminopterin were released from both conjugates by rGGH, indicating that this might by one of the enzymes involved in the release of aminopterin.
i. Incubations of EC2319 and EC1669 with Rat TG Macrophage Cell Lysates, AIA Rat Macrophage, RAW264.7 or THP-1 FRβ Cell Lysates
A. Incubations of EC2319 and EC1669 with Rat TG Macrophage Cell Lysates

50 μL of rat TG macrophage lysate (11.1 million cells/mL PBS) was added to 97 μL of 0.5 M sodium acetate, pH 4.5. To this was added 3 μL of a 50 μM solution of EC2319 or EC1669 (Endocyte, Inc.). The solutions were incubated at 37° C. in a heat block (VWR International, Radnor, Pa.) for 1 hour. At the end of the incubation, the reaction was terminated by the addition of 5 μL of stabilizer solution and 100 μL of acetonitrile containing d₅-aminopterin (Endocyte, Inc.). The rest of the workup is as described above.

B. Incubations of EC2319 and EC1669 with AIA Rat Macrophage, RAW264.7 or THP-1 FRβ Cell Lysates

To 100 μL AIA rat macrophage, RAW264.7 or THP-1 FRβ cell lysates (each containing 10 million cells/mL 0.1 M sodium acetate, Ph 4.5) was added 2 μL of a 50 μM solution of EC2319 or EC1669 (Endocyte, Inc.). The solutions were incubated at 37° C. in a heat block (VWR International, Radnor, Pa.) for 2 hours. The reaction was terminated by the addition of 5 L of stabilizer solution and 100 μL of acetonitrile containing d₅-aminopterin (Endocyte, Inc.). The rest of the workup is as described above.

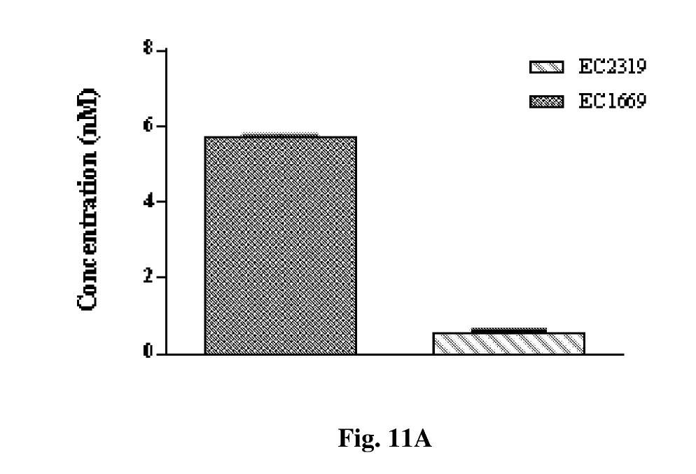

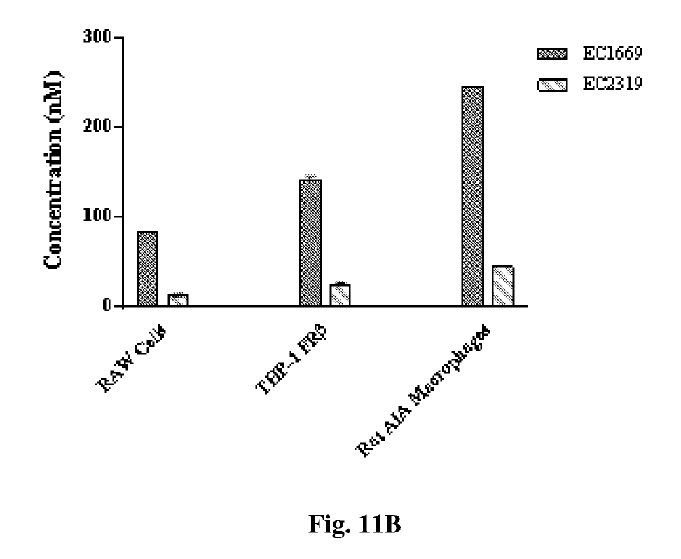

Results: This was done to evaluate if aminopterin could be released from EC1669 and EC2319 in inflammatory and macrophage-like cells from different species. We used cell lysates from RAW264.7 cells (macrophage-like cells from Balb/c mice which express the folate receptors), thioglycollate (TG)-elicited macrophages from rats, macrophages from adjuvant induced arthritic (AIA) rats, and THP-1 cells (human monocytic cells) over-expressing folate receptors. As can be seen from FIGS. 11A and 11B, aminopterin release was observed from both conjugates in all cell lysates, with release being greater from EC1669 than from EC2319.
j. Determination of Plasma Protein Binding:

EC2319 and EC1669 plasma protein binding was evaluated by ultra filtration using VWR 30K MWCO modified PES filters. 250 μL of 500 nM EC2319 or EC1669 in K₃EDTA plasma incubated at 37° C. was added to the upper filter vessel. 50 μL was immediately removed as the donor plasma sample and aliquoted into a clean 1.2 mL plate. The filter apparatus was then centrifuged at 2000×g for 10 minutes to generate plasma ultrafiltrate. An aliquot of 50 L plasma ultrafiltrate was then added to the 1.2 mL plate as the receiver sample. To each plasma sample, 50 μL of plasma ultrafiltrate was added, and each ultrafiltrate sample received 50 μL of plasma to mitigate matrix effects. N-Maleoyl-β-alanine, mannitol, and acetic acid were added at the end of each experiment to stabilize the samples prior to LC-MS/MS analysis.

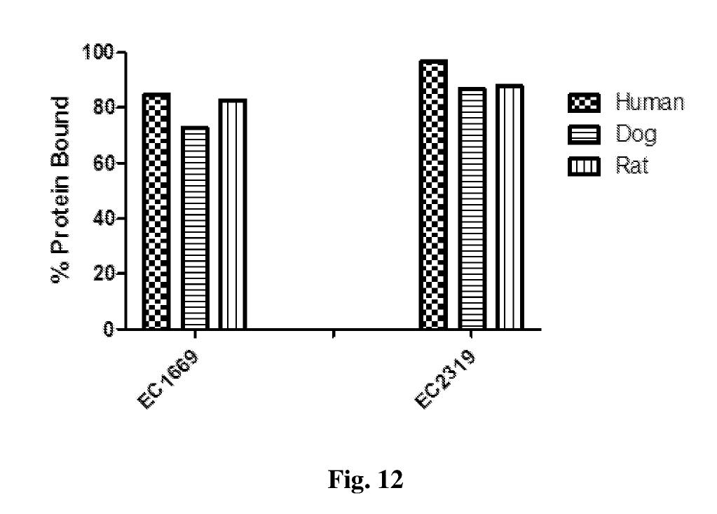

Results: EC2319 and EC1669 plasma protein binding was determined in human, rat, and dog plasma at a 500 nM concentration. As shown in FIG. 12, EC2319 exhibited higher plasma protein binding than did EC1669 in all species tested, although both conjugates exhibit high plasma protein binding.
k. Determination of Whole Blood Stability:

Stability of EC2319 and EC1669 was evaluated in rat and human K₃EDTA blood. Blood samples were maintained at 37° C. for 30 minutes prior to spiking the analyte into 2.5 mL blood at 500 nM. At time 0 and every 30 minutes for two hours, blood samples were removed and centrifuged at 2000×g for 10 minutes to generate plasma. 50 μL aliquots of generated plasma were then transferred to a clean 1.2 mL plate containing N-Maleoyl-β-alanine, mannitol, and acetic acid. Samples were stored at −80° C. until being thawed for LC-MS/MS analysis.

### LC-MS/MS

LC-MS/MS analysis utilized a Waters Acquity UPLC system paired with a Waters Quattro Premier XE tandem quadrupole mass spectrometer operating in ESI positive mode. Prior to injection, K₃EDTA plasma samples were processed using a Phenomenex Strata X-A solid phase extraction (SPE) plate. All plasma samples were fortified with N-Maleoyl-β-alanine, mannitol, and acetic acid. Briefly, 50 μL of plasma diluted with 50 μL of internal standard solution and 500 μL 1000:4:1.5 water:ammonium hydroxide:formic acid was mixed and loaded onto a preconditioned Strata X-A SPE plate. Aqueous and organic washes were then applied followed by elution of all analytes with 300:200:7.5 methanol:water:formic acid. The samples were evaporated until approximately 30 μL remained in all wells and 30 μL of 9:1 water:ammonium hydroxide was then added. Finally, the plate was sealed, mixed, and centrifuged prior to transfer to the 2-8° C. Acquity autosampler. Note: During EC1669 analysis, EC0470 was converted to a hydrazone product for bioanalysis. Acetone (500 μL) was added to all samples after the first evaporation step followed by sealing all wells and heating at 50° C. for one hour. After this step, the acetone was evaporated and the extraction completed as described above.

A 10 μL sample volume was applied to the Waters BEH Shield RP18 100×2.1 mm, 1.7 m UPLC column operating at a flow rate of 0.4 mL/min while being maintained at 45° C. A gradient between mobile phase A (1000:4:1.5 water:ammonium hydroxide:formic acid) and mobile phase B (acetonitrile) was used to separate the analytes. The gradient was held at 2% B for the first 30 seconds of the chromatographic run followed by increasing to 20% B by 2.5 minutes. A rapid gradient from 60-90% B is then used to clean the column prior to equilibration at 2% B to complete the 4 minute run cycle.

For EC1669 MS/MS analysis, EC1669 and its known major metabolites, EC1886 and EC0740, were optimized and monitored. For EC2319 MS/MS analysis, EC2319 and its known major metabolites, EC1886 and EC2496, were optimized and monitored. In all cases, internal standard response was used to generate response ratios for each analyte. When calibrated, data was regressed using MassLynx 4.1 software. The table below lists transitions for analytes and internal standards monitored for LC-MS/MS bioanalysis.

| Analyte | Transition | Internal Standard | Transition |
|---|---|---|---|
| EC1669 | 746.0 > 295.0 | EC1576 | 709.6 > 295.0 |
| EC1886 | 441.0 > 294.0 | EC1886-D5 | 446.0 > 294.2 |
| EC0470 | 455.1 > 294.3 | Methotrexate | 455.2 > 308.3 |
| EC2319 | 755.1 > 294.8 | EC1576 | 709.6 > 295.0 |
| EC2496 | 727.2 > 294.0 | EC1576 | 709.6 > 295.0 |

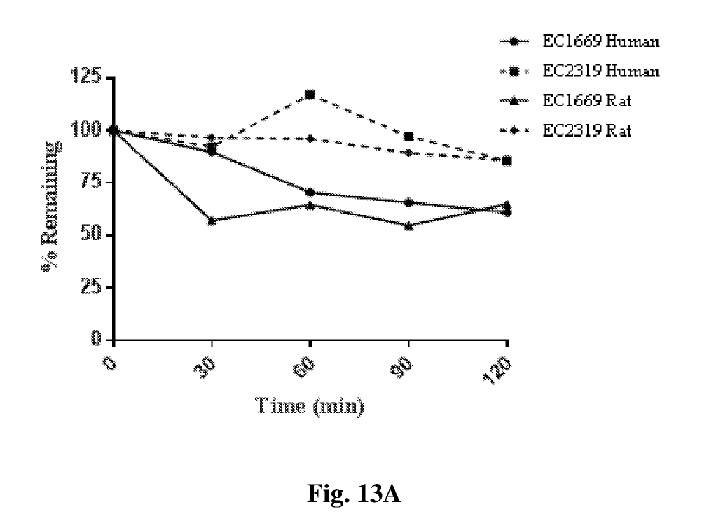

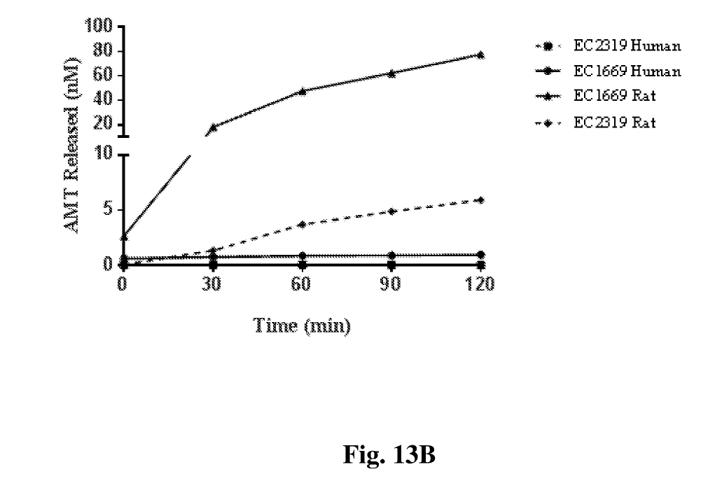

Results: Whole blood stability of EC2319 and EC1669 was evaluated in fresh rat and human blood by monitoring disappearance of intact conjugate and formation of the major metabolite EC1886 over a period of two hours. As shown in FIGS. 13A and 13B, in both species, EC2319 exhibited a superior stability profile than EC1669 based on percent remaining of the intact conjugate as well as the formation of EC1886.

1. A conjugate of the formula B-L-D¹, wherein B is a binding ligand of the formula 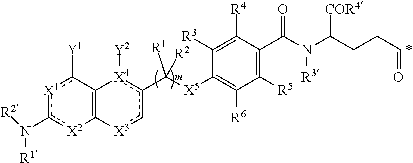
wherein
R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;
X¹ is —NR¹¹—, ═N—, —N═, —C(R¹¹)═ or ═C(R¹¹)—;
X² is —NR^{11′}— or ═N—;
X³ is —NR^{11″}—, —N═ or —C(R^{11′})═;
X⁴ is —N═ or —C═;
X⁵ is NR¹² or CR¹²R^{12′};
Y¹ is H, D, —OR¹³ or —SR¹³ when X¹ is —N═ or —C(R¹¹)═, or Y¹ is ═O when X¹ is —NR¹¹—, ═N— or ═C(R¹¹)—;
Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;
R^{1′}, R^{2′}, R^{3′}, R^{4′}, R¹¹, R^{11′}, R^{11″}, R¹², R^{12′}, R¹³, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};
R¹⁵ and R^{15′} are each independently H or C₁-C₆ alkyl; and
m is 1, 2, 3 or 4;
L is a linker comprising at least one AA, at least one L¹ and an L², wherein each AA is an amino acid, each L¹ is of the formula 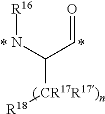
wherein
R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5; and
q is 1, 2, 3, 4 or 5;
and L² is of the formula 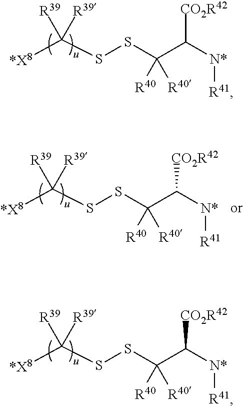
wherein
X⁸ is —NR⁵⁰— or —O—;
each R³⁹, R^{39′}, R⁴⁰ and R^{40′} is independently selected from the group consisting of H, D, C₁-C₆ alkyl, C₂-C₆ alkenyl, C₂-C₆ alkynyl C₃-C₆ cycloalkyl, —OR⁴⁸, —OC(O)R⁴⁸, —OC(O)NR⁴⁸R^{48′}, —OS(O)R⁴⁸, —OS(O)₂R⁴⁸, —SR⁴⁸, —S(O)R⁴⁸, —S(O)₂R⁴⁸, —S(O)NR⁴⁸R^{48′}, —S(O)₂NR⁴⁸R^{48′}, —OS(O)NR⁴⁸R^{48′}, —OS(O)₂NR⁴⁸R^{48′}, —NR⁴⁸R^{48′}, —NR⁴⁸C(O)R⁴⁹, —NR⁴⁸C(O)OR⁴⁹, —NR⁴⁸C(O)NR⁴⁹R^{49′}, —NR⁴⁸S(O)R⁴⁹, —NR⁴⁸S(O)₂R⁴⁹, —NR⁴⁸S(O)NR⁴⁹R^{49′}, —NR⁴⁸S(O)₂NR⁴⁹R^{49′}, —C(O)R⁴⁸, —C(O)OR⁴⁸ or —C(O)NR⁴⁸R^{48′}, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR⁴⁴, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};
each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};
each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′};
each R⁴³, R^{43′}, R⁴⁴, R^{44′}, R⁴⁵, R^{45′}, R⁴⁶, R^{46′}, R⁴⁷, R^{47′}, R⁴⁸, R^{48′}, R⁴⁹, R^{49′} and R⁵⁰ is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl; and
u is 1, 2, 3 or 4; and
D¹ is a drug of the formula 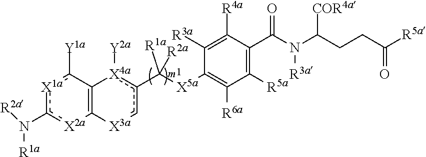
wherein
R^{1a} and R^{2a} in each instance are independently selected from the group consisting of H, D, halogen, C₁-C₆ alkyl, C₂-C₆ alkenyl, C₂-C₆ alkynyl, —OR^{7a}, —SR^{7a} and —NR^{7a}R^{7a′}, wherein each hydrogen atom in C₁-C₆ alkyl, C₂-C₆ alkenyl and C₂-C₆ alkynyl is independently optionally substituted by halogen, —OR^{8a}, —SR^{8a}, —NR^{8a}R^{8a′}, —C(O)R^{8a}, —C(O)OR^{8a} or —C(O)NR^{8a}R^{8a′};
R^{3a}, R^{4a}, R^{5a} and R^{6a} are each independently selected from the group consisting of H, D, halogen, C₁-C₆ alkyl, C₂-C₆ alkenyl, C₂-C₆ alkynyl, —CN, —NO₂, —NCO, —OR^{9a}, —SR^{9a}, —NR^{9a}R^{9a′}, —C(O)R^{9a}, —C(O)OR^{9a} and —C(O)NR^{9a}R^{9a′}, wherein each hydrogen atom in C₁-C₆ alkyl, C₂-C₆ alkenyl and C₂-C₆ alkynyl is independently optionally substituted by halogen, —OR^{10a}, —SR^{10a}, —NR^{10a}R^{10a′}, —C(O)R^{10a}, —C(O)OR^{10a} or —C(O)NR^{10a}R^{10a′};
each R^{7a}, R^{7a′}, R^{8a}, R^{8a′}, R^{9a}, R^{9a′}, R^{10a} and R^{10a′} is independently H, D, C₁-C₆ alkyl, C₂-C₆ alkenyl or C₂-C₆ alkynyl;
X^{1a} is —NR^{11a}—, ═N—, —N═, —C(R^{11a})═ or ═C(R^{11a})—;
X^{2a} is —NR^{11a′}— or ═N—;
X^{3a} is —NR^{11a″}—, —N═ or —C(R^{11a′})═;
X^{4a} is —N═ or —C═;
X^{5a} is —NR^{12a}— or —CR^{12a}R^{12a′}—;
Y^{1a} is —NR^{13a}R^{13a′} when X^{1a} is —N═ or —C(R^{11a})═, or Y^{1a} is ═NR^{13a} when X^{1a} is —NR^{11a}—, ═N— or ═C(R^{11a})—;
Y^{2a} is H, D, C₁-C₆ alkyl, C₂-C₆ alkenyl, —C(O)R^{14a}, —C(O)OR^{14a} or —C(O)NR^{14a}R^{14a′} when X^{4a} is —C═, or Y^{2a} is absent when X^{4a} is —N═;
R^{1a′}, R^{2a′}, R^{3a′}, R^{11a}, R^{11a′}, R^{11a″}, R^{12a}, R^{12a′}, R^{13a}, R^{13a′}, R^{14a} and R^{14a′} are each independently selected from the group consisting of H, D, C₁-C₆ alkyl, C₂-C₆ alkenyl, C₂-C₆ alkynyl, —C(O)R^{15a}, —C(O)OR^{15a} and —C(O)NR^{15a}R^{15a′};
R^{4a′} and R^{5a′} are each independently selected from the group consisting of C₁-C₆ alkyl, C₂-C₆ alkenyl, C₂-C₆ alkynyl, —OR^{16a}, —SR^{16a}, —NR^{16a}R^{16a′}, provided that one of R^{4a′} and R^{5a′} is a covalent bond to an AA, a L¹ or a L²;
R^{15a}, R^{15a′}, R^{16a} and R^{16a′} are each independently H or C₁-C₆ alkyl;
m¹ is 1, 2, 3 or 4; and
each * is a covalent bond;
or a pharmaceutically acceptable salt thereof.

2. A conjugate of the formula B-L-D¹, wherein B is a binding ligand of the formula 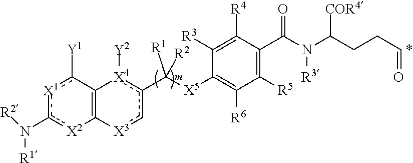
wherein
R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;
X¹ is —NR¹¹—, ═N—, —N═, —C(R¹¹)═ or ═C(R¹¹)—;
X² is —NR^{11′}— or ═N—;
X³ is —NR^{11″}—, —N═ or —C(R^{11′})═;
X⁴ is —N═ or —C═;
X⁵ is NR¹² or CR¹²R^{12′};
Y¹ is H, D, —OR¹³ or —SR¹³ when X¹ is —N═ or —C(R¹¹)═, or Y¹ is ═O when X¹ is —NR¹¹—, ═N— or ═C(R¹¹)—;
Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;
R^{1′}, R^{2′}, R^{3′}, R^{4′}, R¹¹, R^{11′}, R^{11″}, R¹², R^{12′}, R¹³, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};
R¹⁵ and R^{15′} are each independently H or C₁-C₆ alkyl; and
m is 1, 2, 3 or 4;
L is a linker comprising at least one AA, at least one L¹ and an L², wherein each AA is an amino acid, each L¹ is of the formula 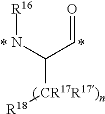
wherein
R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}-(sugar) and —(CH₂)ₚ(OCH₂CH₂CH₂)_{q}(sugar);
R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5; and
q is 1, 2, 3, 4 or 5;
and L² is of the formula 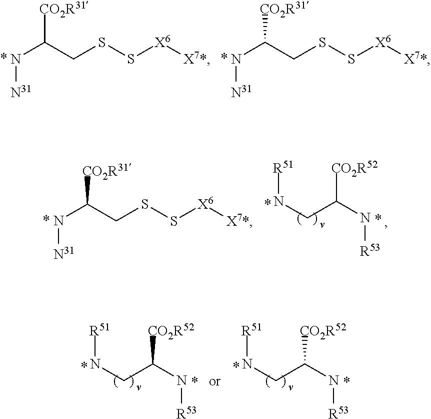
wherein
each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};
each X⁷ is —NR^{31a}— or —O—, and when X⁶ is C₁-C₆ alkyl and X⁷ is —O—, then at least one hydrogen atom in C₁-C₆ alkyl is substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴ or —C(O)NR³⁴R^{34′};
each R³¹ and R^{31a} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};
each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
each R⁵¹ and R⁵³ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁴, —OC(O)R⁵⁴, —OC(O)NR⁵⁴R^{54′}, —OS(O)R⁵⁴, —OS(O)₂R⁵⁴, —SR⁵⁴, —S(O)R⁵⁴, —S(O)₂R⁵⁴, —S(O)NR⁵⁴R^{54′}, —S(O)₂NR⁵⁴R⁵⁴, —OS(O)NR⁵⁴R⁵⁴, —OS(O)₂NR⁵⁴R⁵⁴, —NR⁵⁴R^{54′}, —NR⁵⁴C(O)R⁵⁵, —NR⁵⁴C(O)OR⁵⁵, —NR⁵⁴C(O)NR⁵⁵R⁵⁵, —NR⁵⁴S(O)R⁵⁵, —NR⁵⁴S(O)₂R⁵⁵, —NR⁵⁴S(O)NR⁵⁵R⁵⁵, —NR⁵⁴S(O)₂NR⁵⁵R^{55′}, —C(O)R⁵⁴, —C(O)OR⁵⁴ or —C(O)NR⁵⁴R^{54′};
each R⁵² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁵⁶, —OC(O)R⁵⁶, —OC(O)NR⁵⁶R^{56′}, —OS(O)R⁵⁶, —OS(O)₂R⁵⁶, —SR⁵⁶, —S(O)R⁵⁶, —S(O)₂R⁵⁶, —S(O)NR⁵⁶R^{56′}, —S(O)₂NR⁵⁶R^{56′}, —OS(O)NR⁵⁶R^{56′}, —OS(O)₂NR⁵⁶R^{56′}, —NR⁵⁶R^{56′}, —C(O)R⁵⁶, —C(O)OR⁵⁶ or —C(O)NR⁵⁶R^{56′};
each R⁵⁴, R^{54′}, R⁵⁵, R^{55′}, R⁵⁶ and R^{56′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl; and
v is 1, 2, 3, 4, 5 or 6; and
D¹ is a drug of the formula I 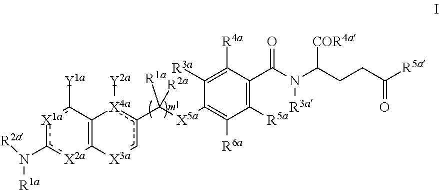
wherein
R^{1a} and R^{2a} in each instance are independently selected from the group consisting of H, D, halogen, C₁-C₆ alkyl, C₂-C₆ alkenyl, C₂-C₆ alkynyl, —OR^{7a}, —SR^{7a} and —NR^{7a}R^{7a′}, wherein each hydrogen atom in C₁-C₆ alkyl, C₂-C₆ alkenyl and C₂-C₆ alkynyl is independently optionally substituted by halogen, —OR^{8a}, —SR^{8a}, —NR^{8a}R^{8a′}, —C(O)R^{8a}, —C(O)OR^{8a} or —C(O)NR^{8a}R^{8a′};
R^{3a}, R^{4a}, R^{5a} and R^{6a} are each independently selected from the group consisting of H, D, halogen, C₁-C₆ alkyl, C₂-C₆ alkenyl, C₂-C₆ alkynyl, —CN, —NO₂, —NCO, —OR^{9a}, —SR^{9a}, —NR^{9a}R^{9a′}, —C(O)R^{9a}, —C(O)OR^{9a} and —C(O)NR^{9a}R^{9a′}, wherein each hydrogen atom in C₁-C₆ alkyl, C₂-C₆ alkenyl and C₂-C₆ alkynyl is independently optionally substituted by halogen, —OR^{10a}, —SR^{10a}, —NR^{10a}R^{10a′}, —C(O)R^{10a}, —C(O)OR^{10a} or —C(O)NR^{10a}R^{10a′};
each R^{7a}, R^{7a′}, R^{8a}, R^{8a′}, R^{9a}, R^{9a′}, R^{10a} and R^{10a′} is independently H, D, C₁-C₆ alkyl, C₂-C₆ alkenyl or C₂-C₆ alkynyl;
X^{1a} is —NR^{11a}—, ═N—, —N═, —C(R^{11a})═ or ═C(R^{11a})—;
X^{2a} is —NR^{11a′}— or ═N—;
X^{3a} is —NR^{11a″}—, —N═ or —C(R^{11a′})═;
X^{4a} is —N═ or —C═;
X^{5a} is —NR^{12a}— or —CR^{12a}R^{12a′}—;
Y^{1a} is H, D, —OR^{13a}, —SR^{13a} or —NR^{13a}R^{13a′} when X^{1a} is —N═ or —C(R^{11a})═, or Y^{1a} is ═NR^{13a} when X^{1a} is —NR^{11a}—, ═N— or ═C(R^{11a})—;
Y^{2a} is H, D, C₁-C₆ alkyl, C₂-C₆ alkenyl, —C(O)R^{14a}, —C(O)OR^{14a} or —C(O)NR^{14a}R^{14a′} when X^{4a} is —C═, or Y^{2a} is absent when X^{4a} is —N═;
R^{1a′}, R^{2a′}, R^{3a′}, R^{11a}, R^{11a′}, R^{11a″}, R^{12a}, R^{12a′}, R^{13a}, R^{13a′}, R^{14a} and R^{14a′} are each independently selected from the group consisting of H, D, C₁-C₆ alkyl, C₂-C₆ alkenyl, C₂-C₆ alkynyl, —C(O)R^{15a}, —C(O)OR^{15a} and —C(O)NR^{15a}R^{15a′};
R^{4a′} and R^{5a′} are each independently selected from the group consisting of C₁-C₆ alkyl, C₂-C₆ alkenyl, C₂-C₆ alkynyl, —OR^{16a}, —SR^{16a}, —NR^{16a}R^{16a′}, provided that one of R^{4a′} and R^{5a′} is a covalent bond to an AA, a L¹ or a L²;
R^{15a}, R^{15a′}, R^{16a} and R^{16a′} are each independently H or C₁-C₆ alkyl;
m¹ is 1, 2, 3 or 4; and
each * is a covalent bond;
or a pharmaceutically acceptable salt thereof.

3. The conjugate of claim 1, having the formula B-L¹-AA-L¹-AA-L¹-L²-D¹, B-AA-L¹-AA-AA-L²-D¹, or B-AA-AA-AA-AA-L²-D¹, or a pharmaceutically acceptable salt thereof.

4. The conjugate of claim 2, or a pharmaceutically acceptable salt thereof, having the formula B-L¹-AA-L¹-AA-L¹-L²-D¹, B-AA-L¹-AA-AA-L²-D¹, or B-AA-AA-AA-AA-L²-D¹.

5.-51. (canceled)

52. The conjugate of claim 1, or a pharmaceutically acceptable salt thereof, wherein L² is of a formula selected from

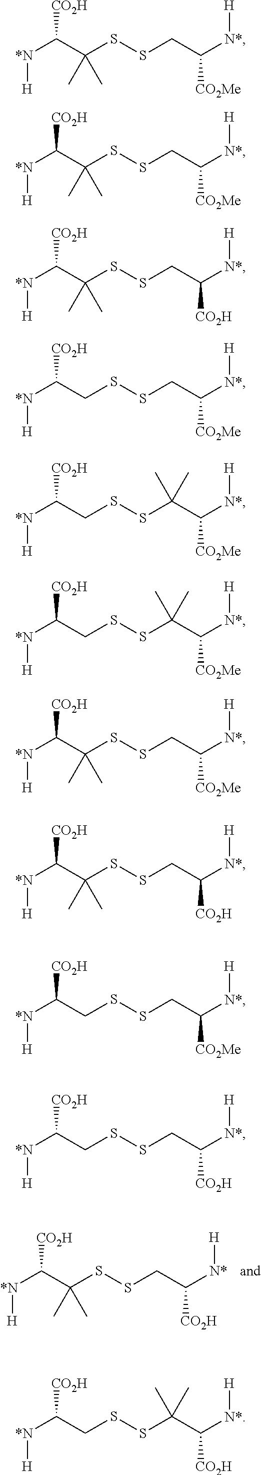

53. The conjugate of claim 52, or a pharmaceutically acceptable salt thereof, wherein L² is of the formula

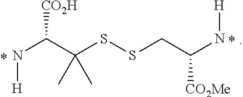 [SMILES: COC(=O)[C@@H](N)CSSC(C)(C)[C@H](N)C(=O)O]

54. The conjugate of claim 2, or a pharmaceutically acceptable salt thereof, wherein L² is of a formula selected from

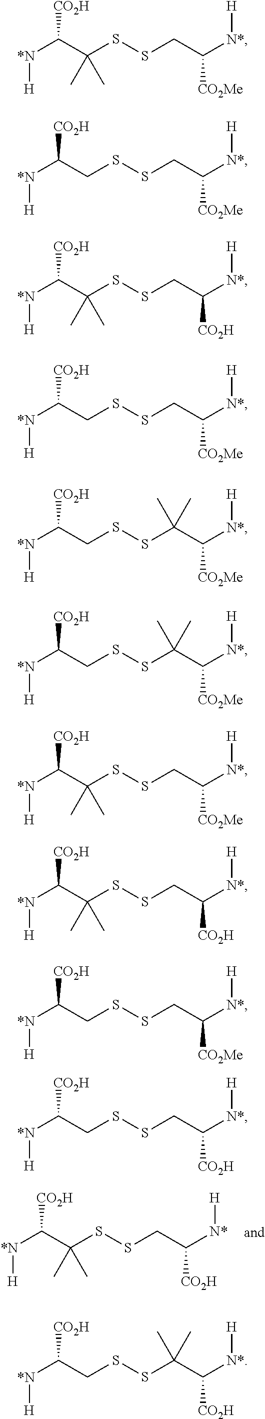

55. The conjugate of claim 2, or a pharmaceutically acceptable salt thereof, wherein L² is of the formula

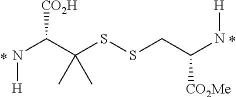 [SMILES: COC(=O)[C@@H](N)CSSC(C)(C)[C@H](N)C(=O)O]

56.-66. (canceled)

67. The conjugate of claim 1, or a pharmaceutically acceptable salt thereof, wherein AA is selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline.

68. The conjugate of claim 2, or a pharmaceutically acceptable salt thereof, wherein AA is selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline.

69. The conjugate of claim 1, selected from the group consisting of 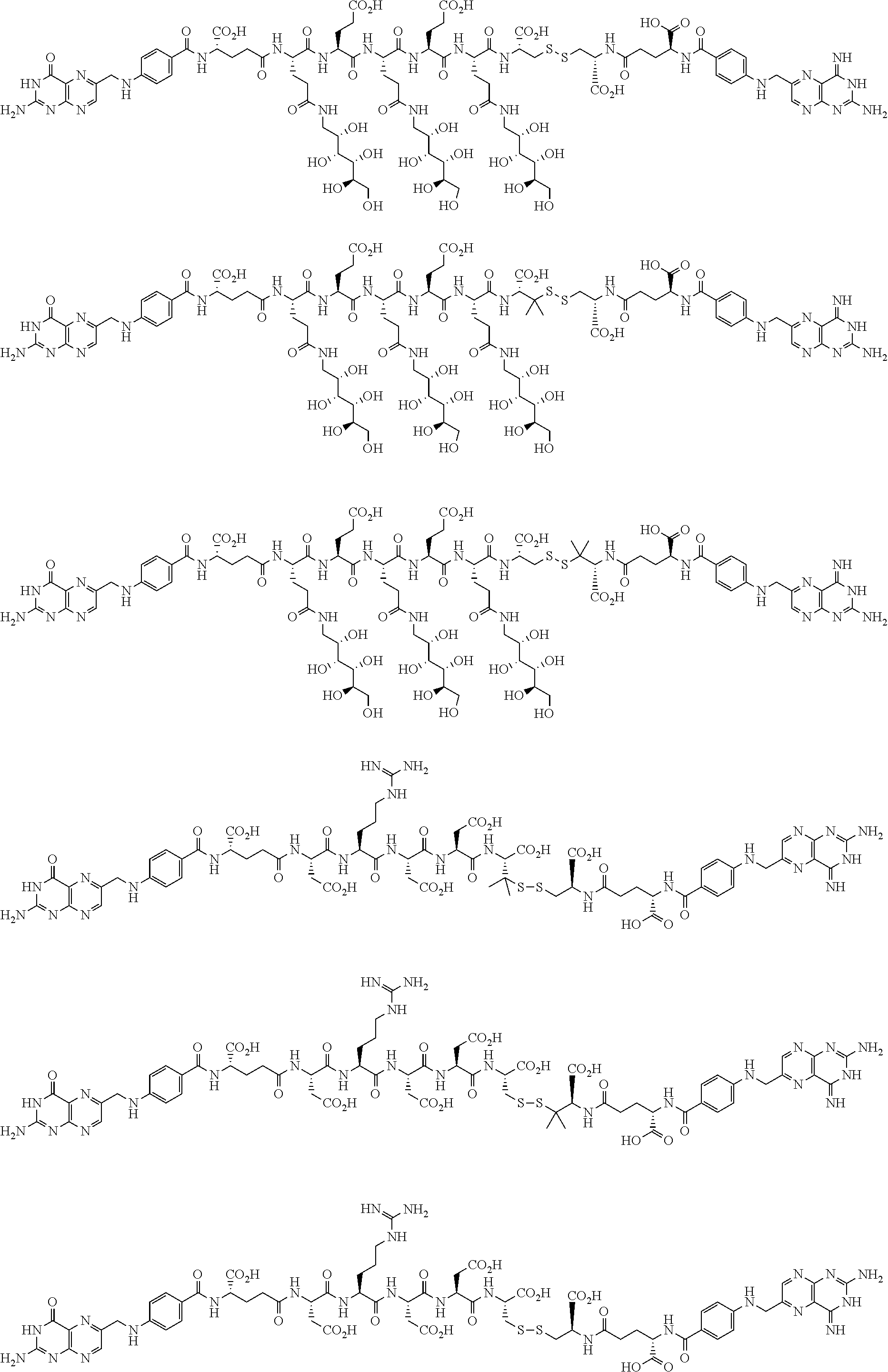 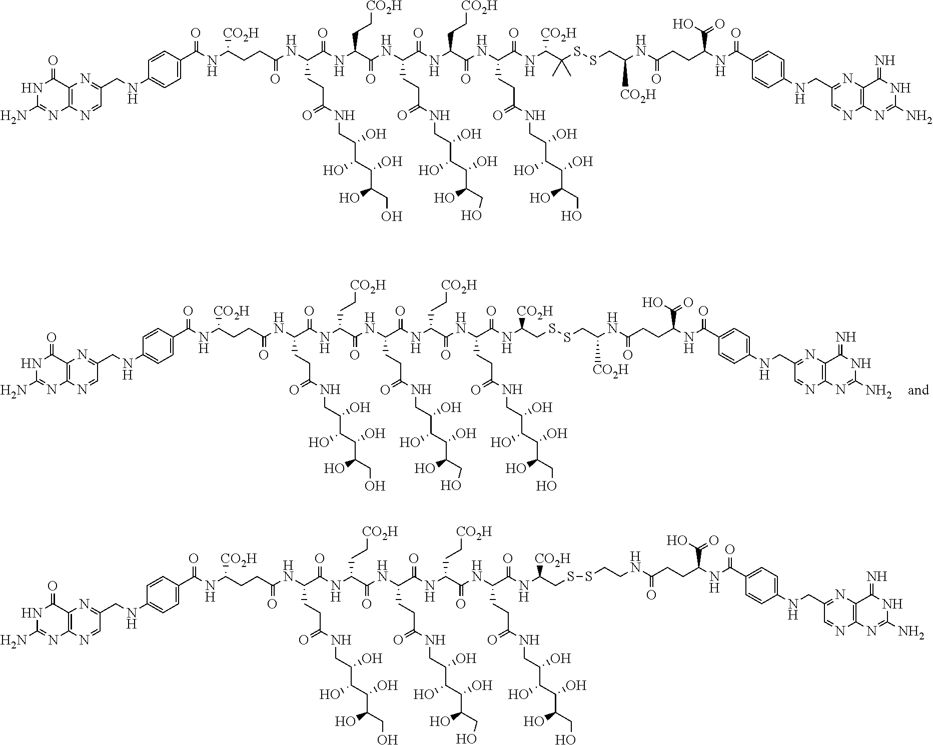
or a pharmaceutically acceptable salt thereof.

70. The conjugate of claim 2, selected from the group consisting of 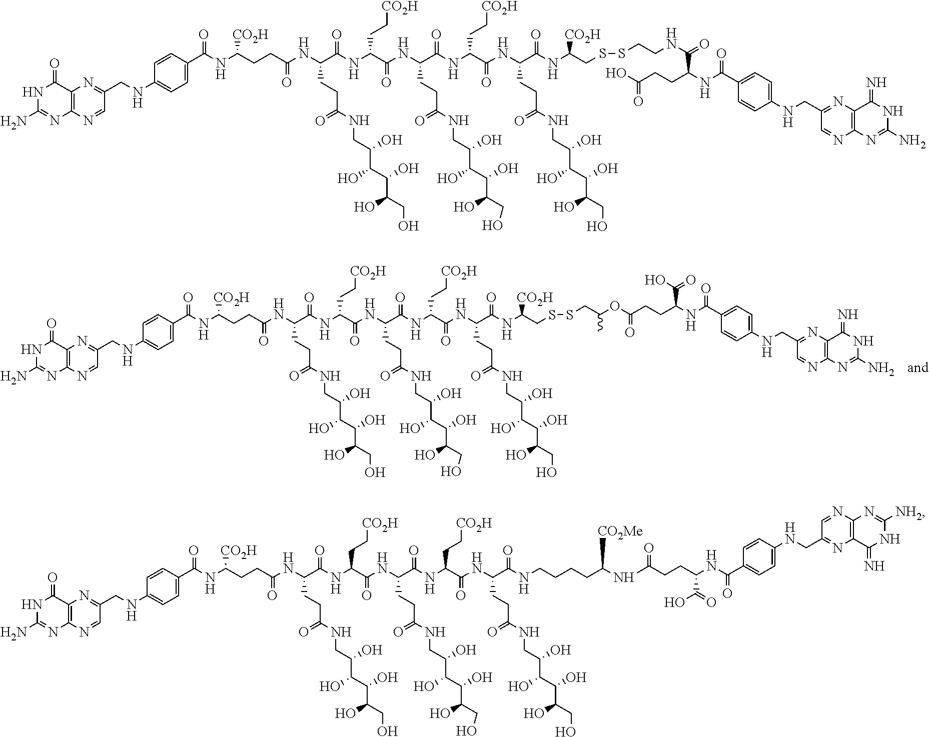
or a pharmaceutically acceptable salt thereof.

71. A pharmaceutical composition comprising a conjugate of claim 1, or a pharmaceutically acceptable salt thereof, and optionally at least one excipient.

72. The pharmaceutical composition of claim 71, wherein the conjugate, or a pharmaceutically acceptable salt thereof, is included in an amount effective to treat disease states caused by inflammatory cells.

73. A method for treating diseases and disease states caused by inflammation comprising administering a therapeutically effective amount of a conjugate of claim 1, or a pharmaceutically acceptable salt thereof, to a patient in need of such treatment.

74. The method of claim 73, wherein the disease caused by inflammation is selected from the group consisting of arthritis, rheumatoid arthritis, osteoarthritis, glomerulonephritis, proliferative retinopathy, restenosis, ulcerative colitis, Crohn's disease, fibromyalgia, psoriasis and other inflammations of the skin, inflammations of the eye, including uveitis and autoimmune uveitis, osteomyelitis, Sögren's syndrome, multiple sclerosis, diabetes, atherosclerosis, pulmonary fibrosis, lupus erythematosus, sarcoidosis, systemic sclerosis, organ transplant rejection (GVHD) and chronic inflammations.

75.-80. (canceled)

81. A pharmaceutical composition comprising a conjugate claim 2, or a pharmaceutically acceptable salt thereof, and optionally at least one excipient.

82. The pharmaceutical composition of claim 81, wherein the conjugate, or a pharmaceutically acceptable salt thereof, is included in an amount effective to treat disease states caused by inflammatory cells.

83. A method for treating diseases and disease states caused by inflammation comprising administering a therapeutically effective amount of a conjugate of claim 2, or a pharmaceutically acceptable salt thereof, to a patient in need of such treatment.

84. The method of claim 83, wherein the disease caused by inflammation is selected from the group consisting of arthritis, rheumatoid arthritis, osteoarthritis, glomerulonephritis, proliferative retinopathy, restenosis, ulcerative colitis, Crohn's disease, fibromyalgia, psoriasis and other inflammations of the skin, inflammations of the eye, including uveitis and autoimmune uveitis, osteomyelitis, Sögren's syndrome, multiple sclerosis, diabetes, atherosclerosis, pulmonary fibrosis, lupus erythematosus, sarcoidosis, systemic sclerosis, organ transplant rejection (GVHD) and chronic inflammations.
